SEQUENCE LISTING (111 sequences)

SEQ ID NO: 1 - DNA, 5441 bp, Artificial Sequence
  CAS9, including a C-terminal SV40 nuclear localization signal, codon pair optimized for expression in S. cerevisiae; the sequence includes the Kl11 promoter from K. lactis and GND2 terminator sequence from S. cerevisiae
ttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgtt

SEQ ID NO: 2 - DNA, 11742 bp, Artificial Sequence
  Plasmid vector pCSN061
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 3 - DNA, 5712 bp, Artificial Sequence
  Plasmid vector pRN1120
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 588 bp, Artificial Sequence
  gBlock of the guide RNA expression cassette to target CAS9 to the INT1 locus
tatagtccgtggaattaattctcatgtttgacagcttatcatcgataatccggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctggggggcccggtacccagcttttgttccctttagtgagggttaattccgagcttggcgtaatcatggtcatagctgtttcctgtgtgaaattgttatccg

SEQ ID NO: 5 - DNA, 588 bp, Artificial Sequence
  gBlock of the guide RNA expression cassette to target CAS9 to the INT59 locus
tatagtccgtggaattaattctcatgtttgacagcttatcatcgataatccggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaaaactcttagcttttccgttttagagctagaaatagcaagttaaaataaggctagtccgttatcaacttgaaaaagtggcaccgagtcggtggtgctttttttgttttttatgtctggggggcccggtacccagcttttgttccctttagtgagggttaattccgagcttggcgtaatcatggtcatagctgtttcctgtgtgaaattgttatccg

SEQ ID NO: 6 - DNA, 588 bp, Artificial Sequence; identical to SEQ ID NO: 5
  gBlock of the guide RNA expression cassette to target CAS9 to the YPRCtau3 locus

SEQ ID NO: 7 - DNA, 20 bp, Artificial Sequence
  guide sequence of the INT1 integration site
tattagaaccagggaggtcc

SEQ ID NO: 8 - DNA, 20 bp, Artificial Sequence
  guide sequence of the INT59 integration site
agaaaactcttagcttttcc

SEQ ID NO: 9 - DNA, 20 bp, Artificial Sequence
  guide sequence of the YPRCtau3 integration site
caatatggtatgccgagtct

SEQ ID NO: 10 - DNA, 77 bp, Artificial Sequence
  FW PCR primer to obtain INT1 SGIC donor DNA sequence for integration, 0 bp deletion
acttctctacattctctgactttttaaaactgtgtactggcgaccaatcgtctttgaaaagataatgtatgattatg

SEQ ID NO: 11 - DNA, 75 bp, Artificial Sequence
  REV PCR primer to obtain INT1 SGIC donor DNA sequence for integration, 0 bp deletion
ggaaattttcaaaggcgttggatcaaaaaataggcctttatttcatcgcgagacataaaaaacaaaaaaagcacc

SEQ ID NO: 12 - DNA, 77 bp, Artificial Sequence
  FW PCR primer to obtain INT1 SGIC donor DNA sequence for integration, 1 kbp deletion
cttcatgccagcaatagttgcgtgctgagctcaacagtgcccaacccttgtctttgaaaagataatgtatgattatg

SEQ ID NO: 13 - DNA, 75 bp, Artificial Sequence
  REV PCR primer to obtain INT1 SGIC donor DNA sequence for integration, 1 kbp deletion
gaaaagcactcctttagtaccactcaacaagttgtctgatgacaaagaatagacataaaaaacaaaaaaagcacc

SEQ ID NO: 14 - DNA, 77 bp, Artificial Sequence
  FW PCRprimer to obtain INT59 SGIC donor DNA sequence for integration, 0 bp deletion
cgccaagagcccaagaaagagacacaaaactacgtgggataagcttggggtctttgaaaagataatgtatgattatg

SEQ ID NO: 15 - DNA, 75 bp, Artificial Sequence
  REV PCR primer to obtain INT59 SGIC donor DNA sequence for integration, 0 bp deletion
tattgcgggattttttggtgccgtacgccggagccgacggaggtaagtcaagacataaaaaacaaaaaaagcacc

SEQ ID NO: 16 - DNA, 77 bp, Artificial Sequence
  FW PCR primer to obtain INT59 SGIC donor DNA sequence for integration, 1 kbp deletion
acgaggtgaagggcaaaggtgaattaaccaaagtgaagaggacgacgtagtctttgaaaagataatgtatgattatg

SEQ ID NO: 17 - DNA, 75 bp, Artificial Sequence
  REV PCR primer to obtain INT59 SGIC donor DNA sequence for integration, 1 kbp deletion
tgatatgagttgtggtgattggagagagtaaaagaaagaatgatagatgcagacataaaaaacaaaaaaagcacc

SEQ ID NO: 18 - DNA, 77 bp, Artificial Sequence
  FW PCR primer to obtain YPRCtau3 SGIC donor DNA sequence for integration, 0 bp deletion
catcttttaatgcctactcttttgatgttcacttacatgttacaatgaagtctttgaaaagataatgtatgattatg

SEQ ID NO: 19 - DNA, 75 bp, Artificial Sequence
  REV PCR primer to obtain YPRCtau3 SGIC donor DNA sequence for integration, 0 bp deletion
gcccctcttatacgattatattaagatccatattcaaccttcattaatacagacataaaaaacaaaaaaagcacc

SEQ ID NO: 20 - DNA, 77 bp, Artificial Sequence
  FW PCR primer to obtain YPRCtau3 SGIC donor DNA sequence for integration, 1 kbp deletion
tcatacgatttccacatgtgtctcatatatattttatgtttaggttaatatctttgaaaagataatgtatgattatg

SEQ ID NO: 21 - DNA, 75 bp, Artificial Sequence
  REV PCR primer to obtain YPRCtau3 SGIC donor DNA sequence for integration, 1 kbp deletion
cttatgtatttttaatcgtccttgtatggaagtatcaaaggggacgttctagacataaaaaacaaaaaaagcacc

SEQ ID NO: 22 - DNA, 488 bp, Artificial Sequence
  INT1 SGIC sequence for integration, 0 bp deletion
acttctctacattctctgactttttaaaactgtgtactggcgaccaatcgtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctcgcgatgaaataaaggcctattttttgatccaacgcctttgaaaatttcc

SEQ ID NO: 23 - DNA, 488 bp, Artificial Sequence
  INT1 SGIC sequence for integration, 1 kb deletion
cttcatgccagcaatagttgcgtgctgagctcaacagtgcccaacccttgtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctattctttgtcatcagacaacttgttgagtggtactaaaggagtgcttttc

SEQ ID NO: 24 - DNA, 488 bp, Artificial Sequence
  INT59 SGIC sequence for integration, 0 bp deletion
cgccaagagcccaagaaagagacacaaaactacgtgggataagcttggggtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaaaactcttagcttttccgttttagagctagaaatagcaagttaaaataaggctagtccgttatcaacttgaaaaagtggcaccgagtcggtggtgctttttttgttttttatgtcttgacttacctccgtcggctccggcgtacggcaccaaaaaatcccgcaata

SEQ ID NO: 25 - DNA, 488 bp, Artificial Sequence
  INT59 SGIC sequence for integration, 1 kb deletion
acgaggtgaagggcaaaggtgaattaaccaaagtgaagaggacgacgtagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaaaactcttagcttttccgttttagagctagaaatagcaagttaaaataaggctagtccgttatcaacttgaaaaagtggcaccgagtcggtggtgctttttttgttttttatgtctgcatctatcattctttcttttactctctccaatcaccacaactcatatca

SEQ ID NO: 26 - DNA, 488 bp, Artificial Sequence
  YPRCtau3 SGIC sequence for integration, 0 bp deletion
catcttttaatgcctactcttttgatgttcacttacatgttacaatgaagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccaatatggtatgccgagtctgttttagagctagaaatagcaagttaaaataaggctagtccgttatcaacttgaaaaagtggcaccgagtcggtggtgctttttttgttttttatgtctgtattaatgaaggttgaatatggatcttaatataatcgtataagaggggc

SEQ ID NO: 27 - DNA, 488 bp, Artificial Sequence
  YPRCtau3 SGIC sequence for integration, 1 kb deletion
tcatacgatttccacatgtgtctcatatatattttatgtttaggttaatatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccaatatggtatgccgagtctgttttagagctagaaatagcaagttaaaataaggctagtccgttatcaacttgaaaaagtggcaccgagtcggtggtgctttttttgttttttatgtctagaacgtcccctttgatacttccatacaaggacgattaaaaatacataag

SEQ ID NO: 28 - DNA, 27 bp, Artificial Sequence
  FW PCR primer annealing to SNR52p to obtain SGIC sequence for integration without genomic flanking regions attached
tctttgaaaagataatgtatgattatg

SEQ ID NO: 29 - DNA, 25 bp, Artificial Sequence
  REV PCR primer annealing to SUP4 3′ flanking region to obtain SGIC sequence for integration without genomic flanking regions attached
agacataaaaaacaaaaaaagcacc

SEQ ID NO: 30 - DNA, 388 bp, Artificial Sequence
  INT1 SGIC without genomic flanking regions attached on either side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtct

SEQ ID NO: 31 - DNA, 388 bp, Artificial Sequence
  INT59 SGIC without genomic flanking regions attached on either side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaaaactcttagcttttccgttttagagctagaaatagcaagttaaaataaggctagtccgttatcaacttgaaaaagtggcaccgagtcggtggtgctttttttgttttttatgtct

SEQ ID NO: 32 - DNA, 388 bp, Artificial Sequence
  YPRCtau3 SGIC without genomic flanking regions attached on either side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccaatatggtatgccgagtctgttttagagctagaaatagcaagttaaaataaggctagtccgttatcaacttgaaaaagtggcaccgagtcggtggtgctttttttgttttttatgtct

SEQ ID NO: 33 - DNA, 25 bp, Artificial Sequence
  FW PCR primer to confirm integration of the SGIC in the INT1 locus, 0 bp deletion.
ttacatttcaggtttccataatatg

SEQ ID NO: 34 - DNA, 24 bp, Artificial Sequence
  REV PCR primer to confirm integration of the SGIC in the INT1 locus, 0 bp deletion
ctttcataagctaattatgccatc

SEQ ID NO: 35 - DNA, 24 bp, Artificial Sequence
  FW PCR primer to confirm integration of the SGIC in the INT1 locus, 1 kbp deletion
tgggttcatactgctgtgttatag

SEQ ID NO: 36 - DNA, 23 bp, Artificial Sequence
  REV PCR primer to confirm integration of the SGIC in the INT1 locus, 1 kbp deletion
aactagctcagaaaacactaacg

SEQ ID NO: 37 - DNA, 19 bp, Artificial Sequence
  FW PCR primer to confirm integration of the SGIC in the INT59 locus, 0 bp deletion
cagacgtaggaggtagccg

SEQ ID NO: 38 - DNA, 19 bp, Artificial Sequence
  REV PCR primer to confirm integration of the SGIC in the INT59 locus, 0 bp deletion
tcacggttctgcggacgcc

SEQ ID NO: 39 - DNA, 22 bp, Artificial Sequence
  FW PCR primer to confirm integration of the SGIC in the INT59 locus, 1 kbp deletion
gaacattaatgactgcaacacc

SEQ ID NO: 40 - DNA, 23 bp, Artificial Sequence
  REV PCR primer to confirm integration of the SGIC in the INT59 locus, 1 kbp deletion
cacaaacggaaaggatatagaag

SEQ ID NO: 41 - DNA, 20 bp, Artificial Sequence
  FW PCR primer to confirm integration of the SGIC in the YPRCtau3 locus, 0 bp deletion
caagtacagtgctgacgtcc

SEQ ID NO: 42 - DNA, 22 bp, Artificial Sequence
  REV primer to confirm integration of the SGIC in the YPRCtau3 locus, 0 bp deletion
attaatgttgaaccaatcggcg

SEQ ID NO: 43 - DNA, 22 bp, Artificial Sequence
  FW PCR primer to confirm integration of the SGIC in the YPRCtau3 locus, 1 kbp deletion
cctctttacaagcggagcttac

SEQ ID NO: 44 - DNA, 22 bp, Artificial Sequence
  REV PCR primer to confirm integration of the SGIC in the YPRCtau3 locus, 1 kbp deletion
gcgcttatttatcggcattgag

SEQ ID NO: 45 - DNA, 77 bp, Artificial Sequence
  FW primer annealing to SNR52p to obtain INT1 SGIC DNA sequence with 50 bp connector sequence at the 5′ end
aagcgacttccaatcgctttgcatatccagtaccacacccacaggcgttttctttgaaaagataatgtatgattatg

SEQ ID NO: 46 - DNA, 75 bp, Artificial Sequence
  REV primer annealing to SUP4 to obtain INT1 SGIC DNA sequence with 50 bp connector sequence at the 3′ end
acttagtatggtctgttggaaaggattgtggcttcgcatacaggctttctagacataaaaaacaaaaaaagcacc

SEQ ID NO: 47 - DNA, 488 bp, Artificial Sequence
  SGIC DNA with connector sequences attached to the 5′ and 3′ ends
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctagaaagcctgtatgcgaagccacaatcctttccaacagaccatactaagt

SEQ ID NO: 48 - DNA, 74 bp, Artificial Sequence
  REV primer annealing to SNR52p to obtain the 5′ split SGIC DNA sequence targeting INT1
attttaacttgctatttctagctctaaaacggacctccctggttctaatagatcatttatctttcactgcggag

SEQ ID NO: 49 - DNA, 73 bp, Artificial Sequence
  FW primer annealing to the guide-RNA to obtain the 3′ split SGIC DNA sequence targeting INT1
aaacttctccgcagtgaaagataaatgatctattagaaccagggaggtccgttttagagctagaaatagcaag

SEQ ID NO: 50 - DNA, 72 bp, Artificial Sequence
  FW primer annealing to the 5′ connector of SGIC DNA fragment to attach genomic DNA sequence for integration on INT1
cttcatgccagcaatagttgcgtgctgagctcaacagtgcccaacccttgaagcgacttccaatcgctttgc

SEQ ID NO: 51 - DNA, 74 bp, Artificial Sequence
  RV primer annealing to the 3′ connector of SGIC DNA fragment to attach genomic DNA sequence for integration on INT1
gaaaagcactcctttagtaccactcaacaagttgtctgatgacaaagaatacttagtatggtctgttggaaagg

SEQ ID NO: 52 - DNA, 588 bp, Artificial Sequence
  SGIC DNA with 50 bp genomic DNA sequences attached on both the 5′ and 3′ end for integration on INT1
cttcatgccagcaatagttgcgtgctgagctcaacagtgcccaacccttgaagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctagaaagcctgtatgcgaagccacaatcctttccaacagaccatactaagtattctttgtcatcagacaacttgttgagtggtactaaaggagtgcttttc

SEQ ID NO: 53 - DNA, 419 bp, Artificial Sequence
  5′ fragment of the split SGIC DNA with 50 bp homology to the 3′ split SGIC DNA cassette for assembly
cttcatgccagcaatagttgcgtgctgagctcaacagtgcccaacccttgaagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaat

SEQ ID NO: 54 - DNA, 249 bp, Artificial Sequence
  3′ fragment of the split SGIC DNA with 50 bp homology to the 5′ split SGIC DNA cassette for assembly.
aaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctagaaagcctgtatgcgaagccacaatcctttccaacagaccatactaagtattctttgtcatcagacaacttgttgagtggtactaaaggagtgcttttc

SEQ ID NO: 55 - DNA, 100 bp, Artificial Sequence
  ssODN 5′ flank 1 kb upper strand sequence
cttcatgccagcaatagttgcgtgctgagctcaacagtgcccaacccttgaagcgacttccaatcgctttgcatatccagtaccacacccacaggcgttt

SEQ ID NO: 56 - DNA, 100 bp, Artificial Sequence
  ssODN 5′ flank 1 kb lower strand sequence
aaacgcctgtgggtgtggtactggatatgcaaagcgattggaagtcgcttcaagggttgggcactgttgagctcagcacgcaactattgctggcatgaag

SEQ ID NO: 57 - DNA, 100 bp, Artificial Sequence
  ssODN 3′ flank 1 kb upper strand sequence
agaaagcctgtatgcgaagccacaatcctttccaacagaccatactaagtattctttgtcatcagacaacttgttgagtggtactaaaggagtgcttttc

SEQ ID NO: 58 - DNA, 100 bp, Artificial Sequence
  ssODN 3′ flank 1 kb lower strand sequence
gaaaagcactcctttagtaccactcaacaagttgtctgatgacaaagaatacttagtatggtctgttggaaaggattgtggcttcgcatacaggctttct

SEQ ID NO: 59 - DNA, 50 bp, Artificial Sequence
  connector sequence on the 5′ end of SGIC DNA
aagcgacttccaatcgctttgcatatccagtaccacacccacaggcgttt

SEQ ID NO: 60 - DNA, 50 bp, Artificial Sequence
  connector sequence on the 3′ end of SGIC DNA
agaaagcctgtatgcgaagccacaatcctttccaacagaccatactaagt

SEQ ID NO: 61 - DNA, 88 bp, Artificial Sequence
  Forward PCR primer SGIC DNA part 5′ fwnA flank-sgRNA-3′ conH
gggggtctcggtgcctgcgacagcggattgggcggagaagaagacaacccttcagatatattcaggtgcttttccctcacatgttttg

SEQ ID NO: 62 - DNA, 87 bp, Artificial Sequence
  Reverse PCR primer SGIC DNA part 5′ fwnA flank-sgRNA-3′ conH
cccggtctcccattaatcgcaactcggatttgggaggcaaggtcggaacgcgaactttggctttgagggtgcggaacatggggactg

SEQ ID NO: 63 - DNA, 36 bp, Artificial Sequence
  Forward PCR primer SGIC DNA hygB or phleo marker-3′ fnwA flank
gggggtctcgaatggctctgtacagtgaccggtgac

SEQ ID NO: 64 - DNA, 86 bp, Artificial Sequence
  Reverse PCR primer SGIC DNA hygB or phleo marker-3′ fnwA flank
cccggtctccgaggcagtcaaagaatcggacagtgcaagttgcgtggtcagccgtcttcttcccatcctgtcttcagtcttaagac

SEQ ID NO: 65 - DNA, 18922 bp, Artificial Sequence
  BG-AMA5 AMA phleo/Cas9 st
cttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtacgcaactctctggaaatgaaggcagccccgaagttttgggatactagcgatcctaggcactgcaccagtcttgaagagggtcatctctccggagattagtccatctgtggcattgtttatactttcacacctccagaacaacatggaagtcaaggaatgtggtatcagactcacaaccaagagatttctcaccaaagcgctagttccaaggcaggtctagcgtgctgacgatggggataatttagccggctaattggtggacatccgccaccaccccagattaaacggtggagatgacagggggcggagattcaacgggattaaatatcggagatgaagactcggcatctgcttgaggcagttagtgcttgatgcaacttgtggtcggtcgaagcgattggcatggtgatcaacgatcggataataagacctcccatgtgcctcgggggatattcgatccgcctgctgaagagagtaatgatggacctgatacttgcagaatctgaactgaagcccttgactagcgctggaactaaatttcaagctaacggtgatgcagcagaaggatgacgatcttttcctaacggatttctccgcagacccccgagcgcattctgcaataccatgcacctttcatgcacctttcatgcaagttccatgcaactcccacacatgtgcattaatatgccttagctctctcgaatgaactttcacgtggcttaagtcccctcacctgcacccatataaaagccaagttcttcccccacgatgacaccaaccccaactcaccttccaccgtcaaaatggacaagaagtatagcatcggtctggacattggcaccaactccgttggctgggctgtcatcaccgacgagtacaaggttcctagcaagaaattcaaggtcctgggaaacaccgatcgtcactccatcaagaagaacctcattggtgcgcttctcttcgactccggtgagactgctgaggccacccgtctgaagcgtaccgctcgccgtcgttacactcgccgcaagaaccgtatctgctacctccaggagattttctccaacgagatggccaaggttgatgactctttcttccaccgtctggaggagtcgttccttgttgaagaagacaagaagcacgagcgtcaccctatcttcggtaacattgtcgatgaggtcgcttaccacgagaagtaccccaccatctaccacctacgcaaaaagctcgtcgacagcaccgacaaggctgacctccgcctcatttacctggctctggcgcacatgatcaagttccgtggtcacttcctgatcgagggtgacctcaaccccgacaactccgatgttgataaactcttcatccagctcgttcagacctacaaccagcttttcgaggaaaaccccatcaacgcgtctggcgtggatgccaaggccatcctctccgctcgcctgagcaagtcccgccgtcttgagaacttgattgcccagctccctggtgagaagaagaacggtcttttcggcaaccttattgccctgtccctcggactgactcccaacttcaagagcaacttcgatcttgctgaggatgctaagttacaactttccaaggatacctacgacgacgaccttgataaccttctcgcccagataggagatcagtacgccgacctcttcctagctgccaagaacctctccgatgccattctcctgtcagatatcctccgtgtcaacactgagatcaccaaggcccctctctccgcctctatgatcaagagatacgatgagcaccaccaggacctcaccctactcaaggctctggtccgccagcagctccccgaaaagtacaaggagatcttcttcgaccagtccaagaacggctacgccggttacatcgacggtggtgcttcccaggaagaattctacaagttcattaagcctatcctcgagaagatggatggcactgaggagcttcttgttaagctgaaccgtgaggaccttctgcgcaagcagcgtactttcgacaacggcagcatcccccaccagatccacctgggtgaattgcacgccatccttcgtcgccaggaagacttctaccctttcttgaaggacaaccgtgagaagattgagaagatcctgaccttccgtatcccctactacgtcggtcctctggctcgcggtaactcccgcttcgcctggatgacccgcaagtccgaggaaaccatcaccccctggaacttcgaggaagtcgtcgacaagggtgcctccgctcagagcttcattgagcgtatgaccaacttcgacaagaacctgcccaacgagaaagtcctgcccaagcactccctcttgtacgagtacttcactgtctacaacgagctgaccaaggtcaagtacgtgaccgagggcatgcgcaagcctgctttcctctccggcgaacagaagaaggccattgtcgacctgctgttcaagactaaccgcaaggtgaccgtcaagcagctcaaggaagactacttcaagaagatcgagtgctttgactccgttgagatctccggtgttgaggaccgcttcaacgcttctctcggcacctaccacgatctgctcaagatcatcaaggacaaggacttccttgacaacgaggagaacgaagacattcttgaggacattgttcttaccctcaccctcttcgaggaccgtgagatgatcgaagaacgtctgaagacctacgctcacctcttcgacgacaaggtcatgaagcagttgaagcgccgccgttacactggctggggtcgcctctctcgcaagttgattaacggtatccgtgataagcagtctggcaagaccatccttgacttcctgaagtccgacggcttcgccaaccgcaacttcatgcagctcatccacgacgactctctgaccttcaaagaggacatccagaaggcccaagtctccggccagggtgactcgctacacgaacacattgccaacctggctggttcccccgctatcaagaagggtatcctgcagactgtgaaggttgttgacgagcttgtgaaggtcatgggtcgtcacaagcccgagaacatcgtcatcgaaatggctcgtgagaaccagaccactcagaagggtcagaagaacagccgtgagcgcatgaagcgtatcgaggaaggcatcaaggagctcggttcccagattctcaaggaacaccccgtcgagaacacccagctgcagaatgagaagctctacctctactacttgcagaacggacgtgacatgtacgtcgaccaggagctggatatcaaccgcctctccgactacgatgttgaccacatcgtcccccagtccttcctcaaggatgacagcattgacaacaaggtgctcacccgttccgacaagaatcgtggcaagagcgataacgtcccctcggaagaggttgttaagaagatgaagaactactggagacaattgctcaacgctaagctcatcactcagcgcaagttcgacaaccttaccaaggccgagcgtggcggactctccgagctcgacaaggccggtttcatcaagcgtcaattggttgaaacccgtcagatcactaagcacgttgcccagatcctggactctcgcatgaacaccaagtacgacgagaacgacaagctcatccgtgaggtcaaggtcatcaccttaaagagcaagctggtcagtgactttaggaaagacttccagttctacaaggtccgcgagatcaacaactaccaccacgctcacgatgcctacctcaacgccgtcgtcggtactgctttgattaagaagtatcccaagctcgagtccgagttcgtctacggtgactacaaggtgtacgacgtgcgcaagatgatcgctaagtccgagcaggagatcggaaaggccactgccaagtacttcttctacagcaacatcatgaacttcttcaagaccgaaataacattggccaacggcgagattcgcaagcgtcccttgattgagactaacggcgaaaccggtgagatcgtctgggacaagggccgtgacttcgctaccgtccgcaaggtcctttctatgccccaggtcaacattgtcaagaagaccgaggtgcagactggtggtttctccaaggagtcgattcttcccaagcgcaactccgacaagctgatcgctcgcaagaaggattgggaccccaagaagtacggtggattcgattcgcctaccgttgcctactccgtcttggttgtcgccaaggtcgagaagggcaagagcaagaagctgaagagtgtgaaggaactcctcggtatcaccatcatggaacgcagcagcttcgagaagaaccctatcgacttcctggaggccaagggttacaaagaggtcaagaaggacctcatcatcaagctccccaagtactctctgttcgagctggagaacggccgtaagcgcatgcttgcttccgccggtgagctccagaagggtaacgagcttgccctcccctccaagtacgtcaacttcctctacctggcctcccactacgagaagctcaagggctctcccgaggacaacgagcagaagcagctctttgtcgagcagcacaagcactacctggatgagatcatcgagcagatctccgagttcagcaagcgtgtcatcctggccgatgccaaccttgacaaggtcctctctgcctacaacaagcaccgtgacaagcccatccgcgagcaggcggagaacatcatccacctgttcaccctcaccaacctgggtgctcctgctgctttcaagtactttgacaccaccatcgaccgcaagcgttacacctccaccaaggaagtgcttgatgcgactctgatccaccagtcgattaccggtctgtacgagactcgtatcgacctgtctcagctcggtggtgactctcgtgccgatcccaagaagaagcgcaaggtttaaacaagcgcttagctcgaacaaaagaaaaagtaaaaacggttaatagcattggattccgaactacaaagtataaactagtttcactccttgtagaagccagatacgggccggggtagataccgcgcactccctcagcagcctcgcagtcatggtcagcatcgaagaactcccacacaaaatgcgctgggagaagagttcgaaatgcagctgggctcaattagccgtcgtacacggagatagatactgaatacatacccgtttgagcctgaaaatttttgacattcgtgcccataccatgaacctcgtac

SEQ ID NO: 66 - DNA, 15186 bp, Artificial Sequence
  BG-AMA9 AMA hygB/Cas9 st./sgRNA cassette
ggtaccttgcccatcgaacgtacaagtactcctctgttctctccttcctttgctttgtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctg

SEQ ID NO: 67 - DNA, 3519 bp, Artificial Sequence
  TOPO Zero Blunt cloning vector
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 68 - DNA, 3506 bp, Artificial Sequence
  Backbone vector AB
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 69 - DNA, 6938 bp, Artificial Sequence
  Vector SGIC DNA hygB
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggttgcccatcgaacgtacaagtactcctctgttctctccttcctttgctttgtgcctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactgcctccggatcgatgtacacaaccgactgcacccaaacgaacacaaatcttagcaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 70 - DNA, 6293 bp, Artificial Sequence
  Vector SGIC DNA phleo
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggttgcccatcgaacgtacaagtactcctctgttctctccttcctttgctttgtgcctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactgcctccggatcgatgtacacaaccgactgcacccaaacgaacacaaatcttagcaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 71 - DNA, 60 bp, Artificial Sequence
  Reverse PCR primer SGIC fragment I
cagtcaaagaatcggacagtgcaagttgcgtggtcagccgtcttcttcccgagggtgcgg

SEQ ID NO: 72 - DNA, 23 bp, Artificial Sequence
  Forward PCR primer SGIC fragment II and III
ctgcgacagcggattgggcggag

SEQ ID NO: 73 - DNA, 22 bp, Artificial Sequence
  Reverse PCR primer SGIC fragment II and IV
cagtcaaagaatcggacagtgc

SEQ ID NO: 74 - DNA, 20 bp, Artificial Sequence
  Reverse PCR primer SGIC fragment III
aatcgcaactcggatttggg

SEQ ID NO: 75 - DNA, 20 bp, Artificial Sequence
  Forward PCR primer SGIC fragment IV
aaagccaaagttcgcgttcc

SEQ ID NO: 76 - DNA, 5174 bp, Artificial Sequence
  TOPO SGIC DNA sgRNA fwnA
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttgggggtctcggtgcctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgattaatgggagaccggg

SEQ ID NO: 77 - DNA, 5997 bp, Artificial Sequence
  TOPO SGIC hygB
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttgggggtctcgaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactgcctcggagaccggg

SEQ ID NO: 78 - DNA, 5352 bp, Artificial Sequence
  TOPO SGIC phleo
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttgggggtctcgaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactgcctcggagaccggg

SEQ ID NO: 79 - DNA, 31 bp, Artificial Sequence
  Forward PCR primer Cas9 with KpnI-flank
cccggtaccgcaactctctggaaatgaaggc

SEQ ID NO: 80 - DNA, 30 bp, Artificial Sequence
  Reverse PCR primer Cas9 with KpnI-flank
cccggtaccgaggttcatggtatgggcacg

SEQ ID NO: 81 - DNA, 14317 bp, Artificial Sequence
  BG-AMA8 AMA hygB / no Cas9 expression cassette
ggtaccttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctg

SEQ ID NO: 82 - DNA, 19242 bp, Artificial Sequence
  BG-AMA14 AMA phleo/Cas9 ++.
cttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtacgcttgcaccaatcgccgtttaggtgttcattaggttgtattttggctatttattgcgatatttaagcgaggcgaacatgggataaaacgtttgctgttagtgtttgtgtatatgctagcgacttagagtgcacgctcaaaagatacccctaacttctgatttgatagcatttgtatatggcatatagtacgcctgaagcataaaaaaaaaaacagaattattatagtttattaatacgagacagcagaatcaccgcccaagttaagcctttgtgctgatcatgctctcgaacgggccaagttcgggaaaagcaaaggagcgtttagtgaggggcaatttgactcacctcccaggcaacagatgaggggggcaaaaagaaagaaattttcgtgagtcaatatggattccgagcatcattttcttgcggtctatcttgctacgtatgttgatcttgacgctgtggatcaagcaacgccactcgctcgctccatcgcaggctggtcgcagacaaattaaaaggcggcaaactcgtacagccgcggggttgtccgctgcaaagtacagagtgataaaagccgccatgcgaccatcaacgcgttgatgcccagctttttcgatccgagaatccaccgtagaggcgatagcaagtaaagaaaagctaaacaaaaaaaaatttctgcccctaagccatgaaaacgagatggggtggagcagaaccaaggaaagagtcgcgctgggctgccgttccggaaggtgttgtaaaggctcgacgcccaaggtgggagtctaggagaagaatttgcatcgggagtggggcgggttacccctccatatccaatgacagatatctaccagccaagggtttgagcccgcccgcttagtcgtcgtcctcgcttgcccctccataaaaggatttcccctccccctcccacaaaattttctttcccttcctctccttgtccgcttcagtacgtatatcttcccttccctcgcttctctcctccatccttctttcatccatctcctgctaacttctctgctcagcacctctacgcattactagccgtagtatctgagcacttctcccttttatattccacaaaacataacaccaccgtcaaaatggacaagaagtatagcatcggtctggacattggcaccaactccgttggctgggctgtcatcaccgacgagtacaaggttcctagcaagaaattcaaggtcctgggaaacaccgatcgtcactccatcaagaagaacctcattggtgcgcttctcttcgactccggtgagactgctgaggccacccgtctgaagcgtaccgctcgccgtcgttacactcgccgcaagaaccgtatctgctacctccaggagattttctccaacgagatggccaaggttgatgactctttcttccaccgtctggaggagtcgttccttgttgaagaagacaagaagcacgagcgtcaccctatcttcggtaacattgtcgatgaggtcgcttaccacgagaagtaccccaccatctaccacctacgcaaaaagctcgtcgacagcaccgacaaggctgacctccgcctcatttacctggctctggcgcacatgatcaagttccgtggtcacttcctgatcgagggtgacctcaaccccgacaactccgatgttgataaactcttcatccagctcgttcagacctacaaccagcttttcgaggaaaaccccatcaacgcgtctggcgtggatgccaaggccatcctctccgctcgcctgagcaagtcccgccgtcttgagaacttgattgcccagctccctggtgagaagaagaacggtcttttcggcaaccttattgccctgtccctcggactgactcccaacttcaagagcaacttcgatcttgctgaggatgctaagttacaactttccaaggatacctacgacgacgaccttgataaccttctcgcccagataggagatcagtacgccgacctcttcctagctgccaagaacctctccgatgccattctcctgtcagatatcctccgtgtcaacactgagatcaccaaggcccctctctccgcctctatgatcaagagatacgatgagcaccaccaggacctcaccctactcaaggctctggtccgccagcagctccccgaaaagtacaaggagatcttcttcgaccagtccaagaacggctacgccggttacatcgacggtggtgcttcccaggaagaattctacaagttcattaagcctatcctcgagaagatggatggcactgaggagcttcttgttaagctgaaccgtgaggaccttctgcgcaagcagcgtactttcgacaacggcagcatcccccaccagatccacctgggtgaattgcacgccatccttcgtcgccaggaagacttctaccctttcttgaaggacaaccgtgagaagattgagaagatcctgaccttccgtatcccctactacgtcggtcctctggctcgcggtaactcccgcttcgcctggatgacccgcaagtccgaggaaaccatcaccccctggaacttcgaggaagtcgtcgacaagggtgcctccgctcagagcttcattgagcgtatgaccaacttcgacaagaacctgcccaacgagaaagtcctgcccaagcactccctcttgtacgagtacttcactgtctacaacgagctgaccaaggtcaagtacgtgaccgagggcatgcgcaagcctgctttcctctccggcgaacagaagaaggccattgtcgacctgctgttcaagactaaccgcaaggtgaccgtcaagcagctcaaggaagactacttcaagaagatcgagtgctttgactccgttgagatctccggtgttgaggaccgcttcaacgcttctctcggcacctaccacgatctgctcaagatcatcaaggacaaggacttccttgacaacgaggagaacgaagacattcttgaggacattgttcttaccctcaccctcttcgaggaccgtgagatgatcgaagaacgtctgaagacctacgctcacctcttcgacgacaaggtcatgaagcagttgaagcgccgccgttacactggctggggtcgcctctctcgcaagttgattaacggtatccgtgataagcagtctggcaagaccatccttgacttcctgaagtccgacggcttcgccaaccgcaacttcatgcagctcatccacgacgactctctgaccttcaaagaggacatccagaaggcccaagtctccggccagggtgactcgctacacgaacacattgccaacctggctggttcccccgctatcaagaagggtatcctgcagactgtgaaggttgttgacgagcttgtgaaggtcatgggtcgtcacaagcccgagaacatcgtcatcgaaatggctcgtgagaaccagaccactcagaagggtcagaagaacagccgtgagcgcatgaagcgtatcgaggaaggcatcaaggagctcggttcccagattctcaaggaacaccccgtcgagaacacccagctgcagaatgagaagctctacctctactacttgcagaacggacgtgacatgtacgtcgaccaggagctggatatcaaccgcctctccgactacgatgttgaccacatcgtcccccagtccttcctcaaggatgacagcattgacaacaaggtgctcacccgttccgacaagaatcgtggcaagagcgataacgtcccctcggaagaggttgttaagaagatgaagaactactggagacaattgctcaacgctaagctcatcactcagcgcaagttcgacaaccttaccaaggccgagcgtggcggactctccgagctcgacaaggccggtttcatcaagcgtcaattggttgaaacccgtcagatcactaagcacgttgcccagatcctggactctcgcatgaacaccaagtacgacgagaacgacaagctcatccgtgaggtcaaggtcatcaccttaaagagcaagctggtcagtgactttaggaaagacttccagttctacaaggtccgcgagatcaacaactaccaccacgctcacgatgcctacctcaacgccgtcgtcggtactgctttgattaagaagtatcccaagctcgagtccgagttcgtctacggtgactacaaggtgtacgacgtgcgcaagatgatcgctaagtccgagcaggagatcggaaaggccactgccaagtacttcttctacagcaacatcatgaacttcttcaagaccgaaataacattggccaacggcgagattcgcaagcgtcccttgattgagactaacggcgaaaccggtgagatcgtctgggacaagggccgtgacttcgctaccgtccgcaaggtcctttctatgccccaggtcaacattgtcaagaagaccgaggtgcagactggtggtttctccaaggagtcgattcttcccaagcgcaactccgacaagctgatcgctcgcaagaaggattgggaccccaagaagtacggtggattcgattcgcctaccgttgcctactccgtcttggttgtcgccaaggtcgagaagggcaagagcaagaagctgaagagtgtgaaggaactcctcggtatcaccatcatggaacgcagcagcttcgagaagaaccctatcgacttcctggaggccaagggttacaaagaggtcaagaaggacctcatcatcaagctccccaagtactctctgttcgagctggagaacggccgtaagcgcatgcttgcttccgccggtgagctccagaagggtaacgagcttgccctcccctccaagtacgtcaacttcctctacctggcctcccactacgagaagctcaagggctctcccgaggacaacgagcagaagcagctctttgtcgagcagcacaagcactacctggatgagatcatcgagcagatctccgagttcagcaagcgtgtcatcctggccgatgccaaccttgacaaggtcctctctgcctacaacaagcaccgtgacaagcccatccgcgagcaggcggagaacatcatccacctgttcaccctcaccaacctgggtgctcctgctgctttcaagtactttgacaccaccatcgaccgcaagcgttacacctccaccaaggaagtgcttgatgcgactctgatccaccagtcgattaccggtctgtacgagactcgtatcgacctgtctcagctcggtggtgactctcgtgccgatcccaagaagaagcgcaaggtttaaacaagcgcttagctcgaacaaaagaaaaagtaaaaacggttaatagcattggattccgaactacaaagtataaactagtttcactccttgtagaagccagatacgggccggggtagataccgcgcactccctcagcagcctcgcagtcatggtcagcatcgaagaactcccacacaaaatgcgctgggagaagagttcgaaatgcagctgggctcaattagccgtcgtacacggagatagatactgaatacatacccgtttgagcctgaaaatttttgacattcgtgcccataccatgaacctcgtac

SEQ ID NO: 83 - DNA, 19569 bp, Artificial Sequence
  BG-AMA17 AMA hygB/Cas9 st
ggtaccgaggttcatggtatgggcacgaatgtcaaaaattttcaggctcaaacgggtatgtattcagtatctatctccgtgtacgacggctaattgagcccagctgcatttcgaactcttctcccagcgcattttgtgtgggagttcttcgatgctgaccatgactgcgaggctgctgagggagtgcgcggtatctaccccggcccgtatctggcttctacaaggagtgaaactagtttatactttgtagttcggaatccaatgctattaaccgtttttactttttcttttgttcgagctaagcgcttgtttaaaccttgcgcttcttcttgggatcggcacgagagtcaccaccgagctgagacaggtcgatacgagtctcgtacagaccggtaatcgactggtggatcagagtcgcatcaagcacttccttggtggaggtgtaacgcttgcggtcgatggtggtgtcaaagtacttgaaagcagcaggagcacccaggttggtgagggtgaacaggtggatgatgttctccgcctgctcgcggatgggcttgtcacggtgcttgttgtaggcagagaggaccttgtcaaggttggcatcggccaggatgacacgcttgctgaactcggagatctgctcgatgatctcatccaggtagtgcttgtgctgctcgacaaagagctgcttctgctcgttgtcctcgggagagcccttgagcttctcgtagtgggaggccaggtagaggaagttgacgtacttggaggggagggcaagctcgttacccttctggagctcaccggcggaagcaagcatgcgcttacggccgttctccagctcgaacagagagtacttggggagcttgatgatgaggtccttcttgacctctttgtaacccttggcctccaggaagtcgatagggttcttctcgaagctgctgcgttccatgatggtgataccgaggagttccttcacactcttcagcttcttgctcttgcccttctcgaccttggcgacaaccaagacggagtaggcaacggtaggcgaatcgaatccaccgtacttcttggggtcccaatccttcttgcgagcgatcagcttgtcggagttgcgcttgggaagaatcgactccttggagaaaccaccagtctgcacctcggtcttcttgacaatgttgacctggggcatagaaaggaccttgcggacggtagcgaagtcacggcccttgtcccagacgatctcaccggtttcgccgttagtctcaatcaagggacgcttgcgaatctcgccgttggccaatgttatttcggtcttgaagaagttcatgatgttgctgtagaagaagtacttggcagtggcctttccgatctcctgctcggacttagcgatcatcttgcgcacgtcgtacaccttgtagtcaccgtagacgaactcggactcgagcttgggatacttcttaatcaaagcagtaccgacgacggcgttgaggtaggcatcgtgagcgtggtggtagttgttgatctcgcggaccttgtagaactggaagtctttcctaaagtcactgaccagcttgctctttaaggtgatgaccttgacctcacggatgagcttgtcgttctcgtcgtacttggtgttcatgcgagagtccaggatctgggcaacgtgcttagtgatctgacgggtttcaaccaattgacgcttgatgaaaccggccttgtcgagctcggagagtccgccacgctcggccttggtaaggttgtcgaacttgcgctgagtgatgagcttagcgttgagcaattgtctccagtagttcttcatcttcttaacaacctcttccgaggggacgttatcgctcttgccacgattcttgtcggaacgggtgagcaccttgttgtcaatgctgtcatccttgaggaaggactgggggacgatgtggtcaacatcgtagtcggagaggcggttgatatccagctcctggtcgacgtacatgtcacgtccgttctgcaagtagtagaggtagagcttctcattctgcagctgggtgttctcgacggggtgttccttgagaatctgggaaccgagctccttgatgccttcctcgatacgcttcatgcgctcacggctgttcttctgacccttctgagtggtctggttctcacgagccatttcgatgacgatgttctcgggcttgtgacgacccatgaccttcacaagctcgtcaacaaccttcacagtctgcaggatacccttcttgatagcgggggaaccagccaggttggcaatgtgttcgtgtagcgagtcaccctggccggagacttgggccttctggatgtcctctttgaaggtcagagagtcgtcgtggatgagctgcatgaagttgcggttggcgaagccgtcggacttcaggaagtcaaggatggtcttgccagactgcttatcacggataccgttaatcaacttgcgagagaggcgaccccagccagtgtaacggcggcgcttcaactgcttcatgaccttgtcgtcgaagaggtgagcgtaggtcttcagacgttcttcgatcatctcacggtcctcgaagagggtgagggtaagaacaatgtcctcaagaatgtcttcgttctcctcgttgtcaaggaagtccttgtccttgatgatcttgagcagatcgtggtaggtgccgagagaagcgttgaagcggtcctcaacaccggagatctcaacggagtcaaagcactcgatcttcttgaagtagtcttccttgagctgcttgacggtcaccttgcggttagtcttgaacagcaggtcgacaatggccttcttctgttcgccggagaggaaagcaggcttgcgcatgccctcggtcacgtacttgaccttggtcagctcgttgtagacagtgaagtactcgtacaagagggagtgcttgggcaggactttctcgttgggcaggttcttgtcgaagttggtcatacgctcaatgaagctctgagcggaggcacccttgtcgacgacttcctcgaagttccagggggtgatggtttcctcggacttgcgggtcatccaggcgaagcgggagttaccgcgagccagaggaccgacgtagtaggggatacggaaggtcaggatcttctcaatcttctcacggttgtccttcaagaaagggtagaagtcttcctggcgacgaaggatggcgtgcaattcacccaggtggatctggtgggggatgctgccgttgtcgaaagtacgctgcttgcgcagaaggtcctcacggttcagcttaacaagaagctcctcagtgccatccatcttctcgaggataggcttaatgaacttgtagaattcttcctgggaagcaccaccgtcgatgtaaccggcgtagccgttcttggactggtcgaagaagatctccttgtacttttcggggagctgctggcggaccagagccttgagtagggtgaggtcctggtggtgctcatcgtatctcttgatcatagaggcggagagaggggccttggtgatctcagtgttgacacggaggatatctgacaggagaatggcatcggagaggttcttggcagctaggaagaggtcggcgtactgatctcctatctgggcgagaaggttatcaaggtcgtcgtcgtaggtatccttggaaagttgtaacttagcatcctcagcaagatcgaagttgctcttgaagttgggagtcagtccgagggacagggcaataaggttgccgaaaagaccgttcttcttctcaccagggagctgggcaatcaagttctcaagacggcgggacttgctcaggcgagcggagaggatggccttggcatccacgccagacgcgttgatggggttttcctcgaaaagctggttgtaggtctgaacgagctggatgaagagtttatcaacatcggagttgtcggggttgaggtcaccctcgatcaggaagtgaccacggaacttgatcatgtgcgccagagccaggtaaatgaggcggaggtcagccttgtcggtgctgtcgacgagctttttgcgtaggtggtagatggtggggtacttctcgtggtaagcgacctcatcgacaatgttaccgaagatagggtgacgctcgtgcttcttgtcttcttcaacaaggaacgactcctccagacggtggaagaaagagtcatcaaccttggccatctcgttggagaaaatctcctggaggtagcagatacggttcttgcggcgagtgtaacgacggcgagcggtacgcttcagacgggtggcctcagcagtctcaccggagtcgaagagaagcgcaccaatgaggttcttcttgatggagtgacgatcggtgtttcccaggaccttgaatttcttgctaggaaccttgtactcgtcggtgatgacagcccagccaacggagttggtgccaatgtccagaccgatgctatacttcttgtccattttgacggtggaaggtgagttggggttggtgtcatcgtgggggaagaacttggcttttatatgggtgcaggtgaggggacttaagccacgtgaaagttcattcgagagagctaaggcatattaatgcacatgtgtgggagttgcatggaacttgcatgaaaggtgcatgaaaggtgcatggtattgcagaatgcgctcgggggtctgcggagaaatccgttaggaaaagatcgtcatccttctgctgcatcaccgttagcttgaaatttagttccagcgctagtcaagggcttcagttcagattctgcaagtatcaggtccatcattactctcttcagcaggcggatcgaatatcccccgaggcacatgggaggtcttattatccgatcgttgatcaccatgccaatcgcttcgaccgaccacaagttgcatcaagcactaactgcctcaagcagatgccgagtcttcatctccgatatttaatcccgttgaatctccgccccctgtcatctccaccgtttaatctggggtggtggcggatgtccaccaattagccggctaaattatccccatcgtcagcacgctagacctgccttggaactagcgctttggtgagaaatctcttggttgtgagtctgataccacattccttgacttccatgttgttctggaggtgtgaaagtataaacaatgccacagatggactaatctccggagagatgaccctcttcaagactggtgcagtgcctaggatcgctagtatcccaaaacttcggggctgccttcatttccagagagttgcggtaccttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctg

SEQ ID NO: 84 - DNA, 13672 bp, Artificial Sequence
  BG-AMA1 AMA phleo / no Cas9 expression cassette
ggtaccttgcccatcgaacgtacaagtactcctctgttctctccttcctttgctttgtgcggagaccggcttactaaaagccagataacagtatgcatatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtttccgttatcggggaagaagtggctgatctcagccaccgcgaaaatgacatcaaaaacgccattaacctgatgttctggggaatataaggtctcgcctccggatcgatgtacacaaccgactgcacccaaacgaacacaaatcttagcagtgccctcgccggatagcttggactgtcctttaccgtcgccagcacaagaagggtatctctgaggtccgtaccgccttttctttaccactggattcgattttcgcagttggaatgatacatctggggactgcgaatggtttacccctcggccgatactatgggtcgtgaagagatggaacattccgaaagtgttttgcggataacattggtggcatcgaaaacagaatgctgaccattgatttcaacacgaacaggaggttgccaagaagcgtacccgccgtgtcgtcaagtcccagcgtgccatcgtcggtgcttccctcgacgtgatcaaggagcgccgctcccagcgccccgaggcccgtgccgccgcccgccagcaggccatcaaggacgccaaggagaagaaggctgccgctgagtccaagaagaaggctgagaaggctaagaacgccgctgctggtgccaagggtgctgctcagcgcatccagagcaagcagggtgctaagggttctgctcccaaggtcgctgccaagtctcgttaaggaatgaataacggttcggcttgggattgggtgcggaaggcaagagtttcatggacgaattttgggaggttactggagctggaatatgtgttttccctaccaccaaaaatgaaatgttccaaaactatcggcgtgcaagacggcctcttacgggtttaacggctctcagataagctctatcaatcgcgccacggatgcatgaatgaagatccagatggccgcgggatatatcgtgctagtgtaattcctacatgatcttgctgttcactccatgcgcatccagatattccaggggtcgactgttaattgatatgcctgggcttgagactccgtagacgcccagtcaatgtgcaattaatacgagggtgctgttatcggcagcaaccttgtacttctccataagatgggggaatgccatggacctgagtgatcaattgacgcaagtctcccataacgcggcggcttgacctaaaatccatataccgccccgttgagcctccgcgctccagagtcctgtcccggaatagggcacaaacctaggctaacctaattcgtcgtccgcgtctgagttcagacaaaagaacttccaagtatcagcagagtacgctgatattgataagtaggcaaacataagaccaataagcaagtagaataaaaaattataaggacactgcctccataaagcgccctcccaagacctcagggacaaaacttctcaagtggcaattcactgcctcaggccgtgtccagtgaagtgacgaagcgacactgttgcctgctgactcagccgctttccgccctgccgaatttgccatctcgcttacaggtcagcactagcgcgattcgcccacagatgctcagcgcaaagtggtgactcagtcaaaccccccctacaagattccacctcgatttttcaacttcccatctcgatccgacaagttctacatccaccgtcaaaatggcctccagcgaagatgtcatcaaggagttcatgcgcttcaaggtccgcatggaaggatccgtcaacggccacgagttcgagattgagggtgagggtgagggccgcccctacgaaggcacccagactgccaagctcaaggtcaccaagggtggtcctctccccttcgcttgggatatcctgtctcctcagttccagtacggctccaaggtctacgtcaagcaccccgccgacatccccgactacaagaagctttctttccccgagggtttcaagtgggagcgtgtcatgaacttcgaggatggtggtgttgtgaccgttactcaggacagcagcttgcaggatggctctttcatctacaaggtcaagttcattggtgtcaacttcccctccgacggccctgtcatgcagaagaagaccatgggctgggaagcgtcgactgagcgtctgtacccccgtgacggtgttctcaagggtgagatccacaaggctctcaagctcaaggacggtggtcactaccttgttgagttcaagtccatctacatggccaagaagcctgtgcagctgcccggatactactacgtggactccaagcttgacatcacctcccacaacgaagactacaccattgttgagcagtacgagcgtgctgagggccgccaccacctcttcctgacccacggaatggatgagctgtacaagtcgaaactataaataaatggtttgcgttgcgattgactgaaacgaaaaaaagcgaaaatgattctgggaatgaattgataaagcgcgggctctgcggtacggttacggttgcggtcgcggacgaatggactgggctgagctgggctggaggaagtccatcgaacaaggacaaggggtggaatatggcacgggtcgattttgttatacataccctaccatccatctatccatttaaataccaaatgagttgttgaatggattcgcggtcttctcggtttatttttgcttgcttgcgtgcttaagggatagtgtgcctcacgctttccggcatcttccagaccacagtatatccatccgcctcctgttgaagcttattttttgtatactgttttgtgatagcacgaagtttttccacggtatcttgttaaaaatatatatttgtggcgggcttacctacatcaaattaataagagactaattataaactaaacacacaagcaagctactttagggtaaaagtttataaatgcttttgacgtataaacgttgcttgtatttattattacaattaaaggtggatagaaaacctagagactagttagaaactaatctcaggtttgcgttaaactaaatcagagcccgagaggttaacagaacctagaaggggactagatatccgggtagggaaacaaaaaaaaaaaacaagacagccacatattagggagactagttagaagctagttccaggactaggaaaataaaagacaatgataccacagtctagttgacaactagatagattctagattgaggccaaagtctctgagatccaggttagttgcaactaatactagttagtatctagtctcctataactctgaagctagaataacttactactattatcctcaccactgttcagctgcgcaaacggagtgattgcaaggtgttcagagactagttattgactagtcagtgactagcaataactaacaaggtattaacctaccatgtctgccatcaccctgcacttcctcgggctcagcagccttttcctcctcattttcatgctcattttccttgtttaagactgtgactagtcaaagactagtccagaaccacaaaggagaaatgtcttaccactttcttcattgcttgtctcttttgcattatccatgtctgcaactagttagagtctagttagtgactagtccgacgaggacttgcttgtctccggattgttggaggaactctccagggcctcaagatccacaacagagccttctagaagactggtcaataactagttggtctttgtctgagtctgacttacgaggttgcatactcgctccctttgcctcgtcaatcgatgagaaaaagcgccaaaactcgcaatatggctttgaaccacacggtgctgagactagttagaatctagtcccaaactagcttggatagcttacctttgccctttgcgttgcgacaggtcttgcagggtatggttcctttctcaccagctgatttagctgccttgctaccctcacggcggatctgcataaagagtggctagaggttataaattagcactgatcctaggtacggggctgaatgtaacttgcctttcctttctcatcgcgcggcaagacaggcttgctcaaattcctaccagtcacaggggtatgcacggcgtacggaccacttgaactagtcacagattagttagcaactagtctgcattgaatggctgtacttacgggccctcgccattgtcctgatcatttccagcttcaccctcgttgctgcaaagtagttagtgactagtcaaggactagttgaaatgggagaagaaactcacgaattctcgacacccttagtattgtggtccttggacttggtgctgctatatattagctaatacactagttagactcacagaaacttacgcagctcgcttgcgcttcttggtaggagtcggggttgggagaacagtgccttcaaacaagccttcataccatgctacttgactagtcagggactagtcaccaagtaatctagataggacttgcctttggcctccatcagttccttcatagtgggaggtccattgtgcaatgtaaactccatgccgtgggagttcttgtccttcaagtgcttgaccaatatgtttctgttggcagagggaacctgtcaactagttaataactagtcagaaactagtatagcagtagactcactgtacgcttgaggcatcccttcactcggcagtagacttcatatggatggatatcaggcacgccattgtcgtcctgtggactagtcagtaactaggcttaaagctagtcgggtcggcttactatcttgaaatccggcagcgtaagctccccgtccttaactgcctcgagatagtgacagtactctggggactttcggagatcgttatcgcgaatgctcggcatactaatcgttgactagtcttggactagtcccgagcaaaaaggattggaggaggaggaggaaggtgagagtgagacaaagagcgaaataagagcttcaaaggctatctctaagcagtatgaaggttaagtatctagttcttgactagatttaaaagagatttcgactagttatgtacctggagtttggatataggaatgtgttgtggtaacgaaatgtaagggggaggaaagaaaaagtcggtcaagaggtaactctaagtcggccattcctttttgggaggcgctaaccataaacggcatggtcgacttagagttagctcagggaatttagggagttatctgcgaccaccgaggaacggcggaatgccaaagaatcccgatggagctctagctggcggttgacaaccccaccttttggcgtttctgcggcgttgcaggcgggactggatacttcgtagaaccagaaaggcaaggcagaacgcgctcagcaagagtgttggaagtgatagcatgatgtgccttgttaactaggtcaaaatctgcagtatgcttgatgttatccaaagtgtgagagaggaaggtccaaacatacacgattgggagagggcctaggtataagagtttttgagtagaacgcatgtgagcccagccatctcgaggagattaaacacgggccggcatttgatggctatgttagtaccccaatggaaagcctgagagtccagtggtcgcagataactccctaaattccctgagctaactctaagtcgaccatgccgtttatggttagcgcctcccaaaaaggaatggccgacttagagttacctcttgaccgactttttctttcctcccccttacatttcgttaccacaacacattcctatatccaaactccaggtacataactagtcgaaatctcttttaaatctagtcaagaactagatacttaaccttcatactgcttagagatagcctttgaagctcttatttcgctctttgtctcactctcaccttcctcctcctcctccaatcctttttgctcgggactagtccaagactagtcaacgattagtatgccgagcattcgcgataacgatctccgaaagtccccagagtactgtcactatctcgaggcagttaaggacggggagcttacgctgccggatttcaagatagtaagccgacccgactagctttaagcctagttactgactagtccacaggacgacaatggcgtgcctgatatccatccatatgaagtctactgccgagtgaagggatgcctcaagcgtacagtgagtctactgctatactagtttctgactagttattaactagttgacaggttccctctgccaacagaaacatattggtcaagcacttgaaggacaagaactcccacggcatggagtttacattgcacaatggacctcccactatgaaggaactgatggaggccaaaggcaagtcctatctagattacttggtgactagtccctgactagtcaagtagcatggtatgaaggcttgtttgaaggcactgttctcccaaccccgactcctaccaagaagcgcaagcgagctgcgtaagtttctgtgagtctaactagtgtattagctaatatatagcagcaccaagtccaaggaccacaatactaagggtgtcgagaattcgtgagtttcttctcccatttcaactagtccttgactagtcactaactactttgcagcaacgagggtgaagctggaaatgatcaggacaatggcgagggcccgtaagtacagccattcaatgcagactagttgctaactaatctgtgactagttcaagtggtccgtacgccgtgcatacccctgtgactggtaggaatttgagcaagcctgtcttgccgcgcgatgagaaaggaaaggcaagttacattcagccccgtacctaggatcagtgctaatttataacctctagccactctttatgcagatccgccgtgagggtagcaaggcagctaaatcagctggtgagaaaggaaccataccctgcaagacctgtcgcaacgcaaagggcaaaggtaagctatccaagctagtttgggactagattctaactagtctcagcaccgtgtggttcaaagccatattgcgagttttggcgctttttctcatcgattgacgaggcaaagggagcgagtatgcaacctcgtaagtcagactcagacaaagaccaactagttattgaccagtcttctagaaggctctgttgtggatcttgaggccctggagagttcctccaacaatccggagacaagcaagtcctcgtcggactagtcactaactagactctaactagttgcagacatggataatgcaaaagagacaagcaatgaagaaagtggtaagacatttctcctttgtggttctggactagtctttgactagtcacagtcttaaacaaggaaaatgagcatgaaaatgaggaggaaaaggctgctgagcccgaggaagtgcagggtgatggcagacatggtaggttaataccttgttagttattgctagtcactgactagtcaataactagtctctgaacaccttgcaatcactccgtttgcgcagctgaacagtggtgaggataatagtagtaagttattctagcttcagagttataggagactagatactaactagtattagttgcaactaacctggatctcagagactttggcctcaatctagaatctatctagttgtcaactagactgtggtatcattgtcttttattttcctagtcctggaactagcttctaactagtctccctaatatgtggctgtcttgttttttttttttgtttccctacccggatatctagtccccttctaggttctgttaacctctcgggctctgatttagtttaacgcaaacctgagattagtttctaactagtctctaggttttctatccacctttaattgtaataataaatacaagcaacgtttatacgtcaaaagcatttataaacttttaccctaaagtagcttgcttgtgtgtttagtttataattagtctcttattaatttgatgtaggtaagcccgccacaaatatatatttttaacaagataccgtggaaaaacttcgtgctatcacaaaacagtatacaaaaaataagctatcgaattcctgcagagatcatcctgtcttcagtcttaagacttctctcctatatcacccgcacttaccctagagtgccgcttaggtgctaagggcacattgagtattggccgtgtagaatatatagcttaagtacggccaagcagacgggaagccctgttctccacaccctatggtcgtatatatcaggcttctaccgggaaacgattaagagtgtataatggactgaaaatcaatatgaacgggacaatgctcaagttaaattagttaggcatcctaatctctactaaatgttctatctagagatcggggtactataggcccgtacgttaatcactctacgcttctctcccttaggtatagtgtaggtaggggctagacatttatatgagtcagatggtacaaacggtaggcagtgcgggcgaagaagtgaagacggagtcggttgaagctacatacaaaagatgcattggctcgtcatgaagagcctcccgggtttagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccattttgacggtgggatcctgtgatgtctgctcaagcggggtagctgttagtcaagctgcgatgaagtgggaaagctcgaactgaaaggttcaaaggaataagggatgggaaggatggagtatggatgtagcaaagtacttacttaggggaaataaaggttcttggatgggaagatgaatatactgaagatgggaaaagaaagagaaaagaaaagagcagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggcgcttattactcagcccttctctctgcgtccgtccgtctctccgcatgccagaaagagtcaccggtcactgtacagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctg

SEQ ID NO: 85 - DNA, 1627 bp, Artificial Sequence
  SGIC DNA fragment I
ctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcgggaagaagacggctgaccacgcaacttgcactgtccgattctttgactg

SEQ ID NO: 86 - DNA, 4081 bp, Artificial Sequence
  SGIC DNA fragment II A
ctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactg

SEQ ID NO: 87 - DNA, 3436 bp, Artificial Sequence
  SGIC DNA fragment II B
ctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactg

SEQ ID NO: 88 - DNA, 1627 bp, Artificial Sequence
  SGIC DNA fragment III
ctgcgacagcggattgggcggagaagaagacaacccttcagatatattcaggtgcttttccctcacatgttttgccgcaccagccatcccactatcaaaaagcgatgatgtttgagattgtcgggtgtccacatcttttagtgtgaatcgctagtagaatttgggatattattgagcatcatcccatgatagcgagtacaagccccgagtaaataccaacattgctatgctgctgtgctgctatctagtttgctacgttggtcgttgacctcacagggatttccaccaaaaagtggaccgggcgggcgccactcggccgtgccacagcagcctgagagcggacaaataacaacagccgcctgccgcggggttcggttgcaaacatgaccaacaggccaggccatcatcaacccaccgctgcgttgatgcccaggatttcagtccaataatccacaatttaccaacggatagagctaggtgaattagatagacaggagggccagagggaggggaccgagatgaaaaattttcgatgaaagagtggtcaaggtggggtcgtagttcggcgctccgagggcgaggaaccaaggaaaggcgaggaaaggacaggctgatcgcgctgcgttgctgggctgcaagcgtgtccagttgagtctggaaaaggctccgccgtgaagattctgcgttggtcccgcacctgcgcggtgggggcattacccctccatgtccaatgatttcaagtcaaagccaagggttgaagcccgcccgcttagtcgccttctcgcttgacccctccatataagtatttcccctcctccccctcccacaaatttttcctttccctttcctccctcgtccgcttcagtacgtatatcttccccccctctctcttccttctcactcttctctccttctttcttgattcatcctctctctaactgacttctttgctcagcacctctacgcgttctggccgtagtatctgagcaatttttctacagactttttctatctaattccaaaaaagaacttcgagttcattcaccaccgtcaaaatgatctgactgatgagtccgtgaggacgaaacgagtaagctcgtctcagatatattcagtcactggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaaatgcgctaaactgggcttgactcagggagggatcatggactagccaattgggcgtgcacagcgcgactttggagctggttctggctcgcatgacttgtttcgtgctgcgggggattccgttcggacctgacattttaaaaataaaaaatggaaacatcttgaaagacaaaaatgagtttcagtagtggtctacagaccgtagttttgttcctattcacagtgaaaataaggcgctgcaattgctacgttcataaatcgagtattgttgtgctccgaagcgccagtccccatgttccgcaccctcaaagccaaagttcgcgttccgaccttgcctcccaaatccgagttgcgatt

SEQ ID NO: 89 - DNA, 2504 bp, Artificial Sequence
  SGIC DNA fragment IV A
aaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactg

SEQ ID NO: 90 - DNA, 1859 bp, Artificial Sequence
  SGIC DNA fragment IV B
aaagccaaagttcgcgttccgaccttgcctcccaaatccgagttgcgattaatggctctgtacagtgaccggtgactctttctggcatgcggagagacggacggacgcagagagaagggctgagtaataagcgccactgcgccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcacaggatcccaccgtcaaaatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaacccgggaggctcttcatgacgagccaatgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggagagaagtcttaagactgaagacaggatgggaagaagacggctgaccacgcaacttgcactgtccgattctttgactg

SEQ ID NO: 91 - DNA, 388 bp, Artificial Sequence
  Nucleotide sequence of the gBlock that contains the sgRNA expression cassette to target ORF1; i.e. ORF1_SGIC DNA before the genomic flanking regions are added to either 5′ and 3′ end
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccctataccaattcctatggtgttttagagctagaaatagcaagttaaaataaggctagtccgttatcaacttgaaaaagtggcaccgagtcggtggtgctttttttgttttttatgtct

SEQ ID NO: 92 - DNA, 388 bp, Artificial Sequence
  Nucleotide sequence of the gBlock that contains the sgRNA expression cassette to target ORF2; i.e. ORF2_SGIC DNA before the genomic flanking regions are added to either 5′ and 3′ end
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgatgagcgtggtaaccgattgttttagagctagaaatagcaagttaaaataaggctagtccgttatcaacttgaaaaagtggcaccgagtcggtggtgctttttttgttttttatgtct

SEQ ID NO: 93 - DNA, 388 bp, Artificial Sequence
  Nucleotide sequence of the gBlock that contains the sgRNA expression cassette to target ORF3; i.e. ORF3_SGIC DNA before the genomic flanking regions are added to either 5′ and 3′ end
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgacccaatgctggccaccgggttttagagctagaaatagcaagttaaaataaggctagtccgttatcaacttgaaaaagtggcaccgagtcggtggtgctttttttgttttttatgtct

SEQ ID NO: 94 - DNA, 20 bp, Artificial Sequence
  Nucleotide sequence of the guide sequence (genomic target sequence) of ORF1
cctataccaattcctatggt

SEQ ID NO: 95 - DNA, 20 bp, Artificial Sequence
  Nucleotide sequence of the guide sequence (genomic target sequence) of ORF2
gatgagcgtggtaaccgatt

SEQ ID NO: 96 - DNA, 20 bp, Artificial Sequence
  Nucleotide sequence of the guide sequence (genomic target sequence) of ORF3
gacccaatgctggccaccgg

SEQ ID NO: 97 - DNA, 77 bp, Artificial Sequence
  Nucleotide sequence of the forward primer to obtain ORF1 SGIC DNA sequence for integration
acgaagacgtttatagacataaataaagaggaaacgcattccgtggtagatctttgaaaagataatgtatgattatg

SEQ ID NO: 98 - DNA, 75 bp, Artificial Sequence
  Nucleotide sequence of the reverse primer to obtain ORF1 SGIC DNA sequence for integration
tcctgtcattaagagtttttattttttattataatactcaacacgtgactagacataaaaaacaaaaaaagcacc

SEQ ID NO: 99 - DNA, 77 bp, Artificial Sequence
  Nucleotide sequence of the forward primer to obtain ORF2 SGIC DNA sequence for integration
ccagcgtatacaatctcgatagttggtttcccgttctttccactcccgtctctttgaaaagataatgtatgattatg

SEQ ID NO: 100 - DNA, 75 bp, Artificial Sequence
  Nucleotide sequence of the reverse primer to obtain ORF2 SGIC DNA sequence for integration
gtttttataacgttcgctgcactgggggccaagcacagggcaagatgcttagacataaaaaacaaaaaaagcacc

SEQ ID NO: 101 - DNA, 77 bp, Artificial Sequence
  Nucleotide sequence of the forward primer to obtain ORF3 SGIC DNA sequence for integration
gcggcttcagccgttctgaaccttcaagatggtgttcgggtgtgatttattctttgaaaagataatgtatgattatg

SEQ ID NO: 102 - DNA, 75 bp, Artificial Sequence
  Nucleotide sequence of the reverse primer to obtain ORF3 SGIC DNA sequence for integration
attatccaaacaaagggtctttcgttagcaaacctagaaatctgcaaaaaagacataaaaaacaaaaaaagcacc

SEQ ID NO: 103 - DNA, 488 bp, Artificial Sequence
  Nucleotide sequence of ORF1 SGIC DNA with genomic flanking regions attached at both the 5′ and 3′ end to either side for integration
acgaagacgtttatagacataaataaagaggaaacgcattccgtggtagatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatccctataccaattcctatggtgttttagagctagaaatagcaagttaaaataaggctagtccgttatcaacttgaaaaagtggcaccgagtcggtggtgctttttttgttttttatgtctagtcacgtgttgagtattataataaaaaataaaaactcttaatgacagga

SEQ ID NO: 104 - DNA, 488 bp, Artificial Sequence
  Nucleotide sequence of ORF2 SGIC DNA with genomic flanking regions attached at both the 5′ and 3′ end to either side for integration
ccagcgtatacaatctcgatagttggtttcccgttctttccactcccgtctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgatgagcgtggtaaccgattgttttagagctagaaatagcaagttaaaataaggctagtccgttatcaacttgaaaaagtggcaccgagtcggtggtgctttttttgttttttatgtctaagcatcttgccctgtgcttggcccccagtgcagcgaacgttataaaaac

SEQ ID NO: 105 - DNA, 488 bp, Artificial Sequence
  Nucleotide sequence of ORF3 SGIC DNA with genomic flanking regions attached at both the 5′ and 3′ end to either side for integration
gcggcttcagccgttctgaaccttcaagatggtgttcgggtgtgatttattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgacccaatgctggccaccgggttttagagctagaaatagcaagttaaaataaggctagtccgttatcaacttgaaaaagtggcaccgagtcggtggtgctttttttgttttttatgtcttttttgcagatttctaggtttgctaacgaaagaccctttgtttggataat

SEQ ID NO: 106 - DNA, 24 bp, Artificial Sequence
  Nucleotide sequence of forward primer to confirm knock out of ORF1 by integration of ORF1 SGIC DNA
tccctataccaattcctatggtgt

SEQ ID NO: 107 - DNA, 20 bp, Artificial Sequence
  Nucleotide sequence of reverse primer to confirm knock out of ORF1 by integration of ORF1 SGIC DNA
tggttcagttcacagggctt

SEQ ID NO: 108 - DNA, 20 bp, Artificial Sequence
  Nucleotide sequence of forward primer to confirm knock out of ORF2 by integration of ORF2 SGIC DNA
atcgatgagcgtggtaaccg

SEQ ID NO: 109 - DNA, 20 bp, Artificial Sequence
  Nucleotide sequence of reverse primer to confirm knock out of ORF2 by integration of ORF2 SGIC DNA
cgcatgcacgaaaaagggaa

SEQ ID NO: 110 - DNA, 19 bp, Artificial Sequence
  Nucleotide sequence of forward primer to confirm knock out of ORF3 by integration of ORF3 SGIC DNA
tgatcgacccaatgctggc

SEQ ID NO: 111 - DNA, 22 bp, Artificial Sequence
  Nucleotide sequence of reverse primer to confirm knock out of ORF3 by integration of ORF3 SGIC DNA
tcttcttgaaccatgaacccgt

(19) United States
(12) Patent Application Publication
(10) Pub. No.: US 2020/0032252 A1
(43) Pub. Date: Jan. 30, 2020

(54) SELF-GUIDING INTEGRATION CONSTRUCT (SGIC)

(71) Applicant: DSM IP ASSETS B.V., Heeerlen (NL)

(72) Inventors: Johannes Andries ROUBOS, Echt (NL); René VERWAAL, Echt (NL); Bianca Elisabeth Maria GIELESEN, Echt (NL); Brenda VONK, Echt (NL)

(21) Appl. No.: 16/500,717

(22) PCT Filed: Apr. 4, 2018
(86) PCT No.: PCT/EP2018/058612

§ 371 Date: Oct. 3, 2019

(30) Foreign Application Priority Data
Apr. 6, 2017 (EP) 17165201.9

(51) Int. Cl. C12N 15/11; C12N 15/81; C12N 15/90; C12N 9/22

(52) U.S. Cl. CPC C12N 15/11; C12N 15/81; C12N 2310/20; C12N 9/22; C12N 2800/80; C12N 15/905

(57) ABSTRACT

The present invention relates to the field of molecular biology and cell biology. More specifically, the present invention relates to a self-guiding integration construct for a genome editing system.

### FIELD

The present invention relates to the field of molecular biology and cell biology. More specifically, the present invention relates to a self-guiding integration construct for a genome editing system.

### BACKGROUND

A polynucleotide-guided nuclease system, also referred to as polynucleotide-guided genome editing system, from which the best known is the CRISPR/Cas9 system, is a powerful tool that has been leveraged for genome editing and gene regulation. This tool requires at least a polynucleotide-guided nuclease such as Cas9 and a guide-polynucleotide such as a guide-RNA that enables the genome editing enzyme to target a specific sequence of DNA. In addition, for editing of the genome in a precise way, a donor polynucleotide such as a donor DNA is mostly required, especially when relying on homologous recombination for editing precisely at a desired spot in the genome instead of relying on repair by a random repair process, such as non-homologous end joining. For each target site, a donor polynucleotide needs to be designed and synthesized. In addition, a guide-polynucleotide specific for a target site in the genome needs to be designed and needs to be expressed within the cell or needs to be expressed in vitro and introduced into the cell. For targeted modification with the CRISPR/Cas9 system, a combination of a guide-polynucleotide and a donor polynucleotide which are specific for a target need to be used. Especially for multiplex approaches such as when screening, e.g., a knock-out library, a knock-down library or a promoter-replacement library, the experimental work is quite laborious since matching compositions comprising a guide-polynucleotide or guide-polynucleotide expression construct and a matching donor polynucleotide will have to be transformed together. For screening multiple targets and/or multiple modifications in one experiment, the state of the art set-up requires a multiplex of polynucleotides to be added and used and an even higher amount of screenings for a cell comprising the desired properties. Accordingly, there is a continuing urge to develop improved and simplified guide-polynucleotide and donor-polynucleotide tools.

### BRIEF DESCRIPTION OF THE DRAWINGS

FIG. 1 depicts the vector map of single copy (CEN/ARS) vector pCSN061 expressing Cas9 codon-pair optimized for expression in S. cerevisiae. CPO Cas9 is expressed from the Kluyveromyces lactis KLLA0F20031g promoter and the S. cerevisiae GND2 terminator.

A KanMX marker cassette is present on the vector, which confers resistance against G418 to allow selection of transformants on plate or in liquid cultures. The TRP1 marker allows selection of the plasmid in yeast strains with a trp1 auxotrophy.

FIG. 2 depicts the vector map of multi-copy (2 micron) vector pRN1120. A NatMX marker cassette is present on the vector, which confers resistance against nourseothricin to allow selection of transformants on plate or in liquid cultures. The vector is used for used for in vivo recombination of an sgRNA expression cassette after linearization using EcoRI and XhoI.

FIGS. 3A-3D depict the integration of a Self-Guiding Integration Construct (SGIC) type guide-RNA expression cassette using a CRISPR/Cas9 system in Saccharomyces cerevisiae as described in Example 1. The SGIC's comprise 50 bp flanks at both the 5′ and 3′ end with sequence identity with genomic DNA sequences to allow integration via homologous recombination at the desired genomic locus (either INT1, INT59 or YPRCtau3). Depending on the sequence of the flanks, a stretch of DNA of up to 1 kbp is deleted from the genome upon integration of the SGIC. FIG. 3A: no flank control; FIG. 3B: 0 kB deletion; FIG. 3C: 1 kB deletion; FIG. 3D: no SGIC fragment.

FIGS. 4A-4C depict two SGIC split guide-RNA fragments which are essentially two halves of an SGIC as set forward in Example 1 having a 80 bp overlap homology with each other to allow in vivo (within a yeast cell) assembly of the functional SGIC. The assembled functional SGIC guide-RNA comprised a guide-RNA expression cassette and 50 bp flanks at both the 5′ and 3′ end with sequence identity with genomic DNA sequences to allow integration via homologous recombination at the desired genomic locus. The functional SGIC comprising the guide-RNA expression cassette was subsequently integrated into the INT1 locus of the S. cerevisiae genome. Grey boxes that are part of the split SGIC or sgRNA constructs represent sequences homologous to genomic DNA of the INT1 locus. Black boxes that are part of the split SGIC or sgRNA constructs represent connector sequences (50 bp DNA sequences with no homology to S. cerevisiae genomic DNA). FIG. 4A: Split SGIC; FIG. 4B: SGIC with separate ssODN flanks; FIG. 4C; SGIC DNA with flanks attached.

FIG. 5 depicts the map of vector BG-AMA5 expressing Cas9 codon-pair optimized for expression in A. niger and is used in Example 3. Details of the vector and its construction are described in WO2016110453A1.

FIG. 6 depicts the map of vector BG-AMA9 for expression in A. niger and is used in Example 3. Details of the vector and its construction are described in WO2016110453A1.

FIG. 7 depicts the map of vector SGIC DNA hygB used in Example 3.

FIG. 8 depicts the map of vector SGIC DNA phleo used in Example 3.

FIGS. 9A-9C depict experiment 3 that exemplifies the use of SGIC to disrupt the fwnA6 gene in Aspergillus niger as further detailed in the description of example 3 and in Tables 10-15.

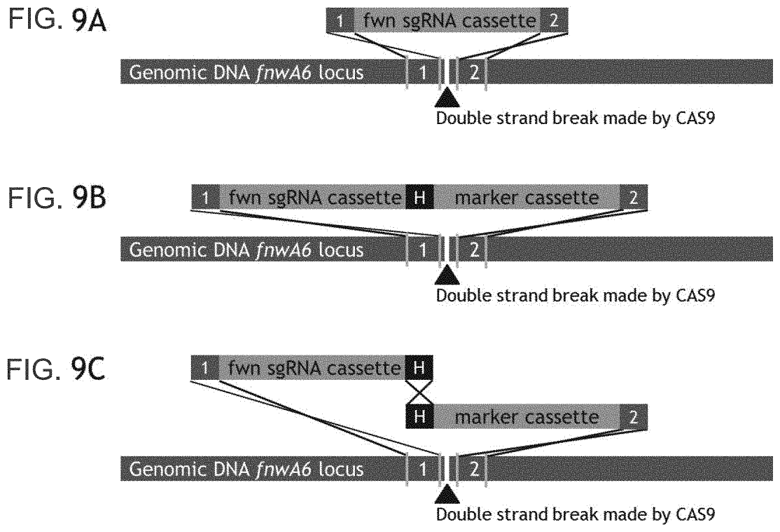

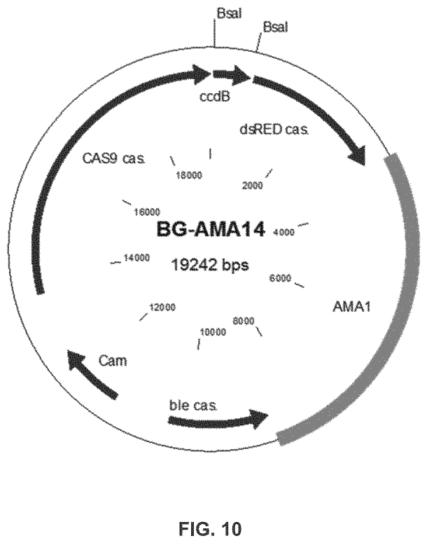

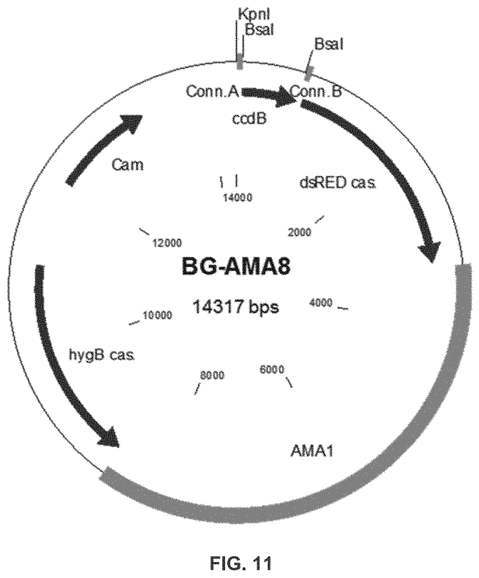

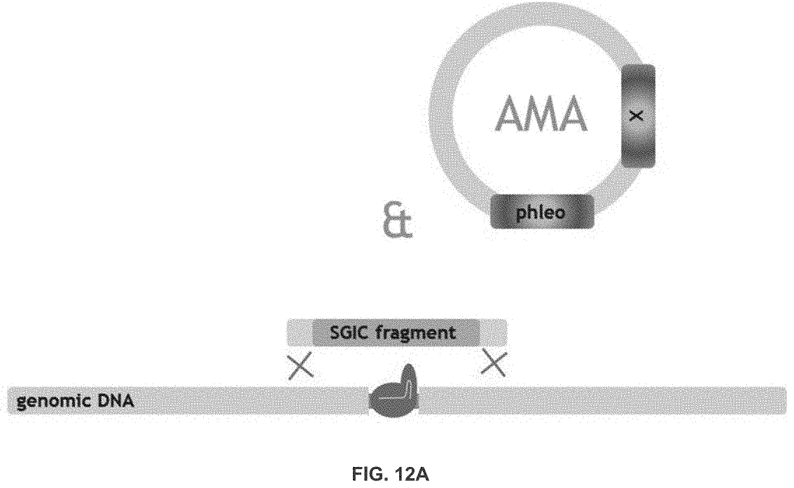

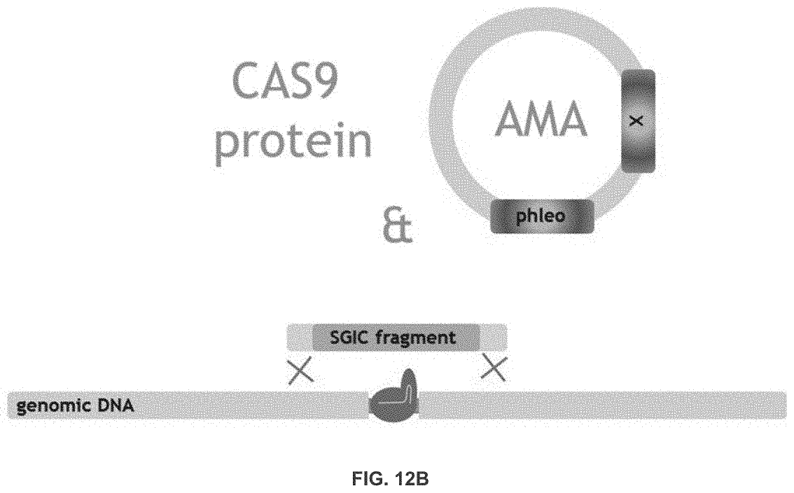

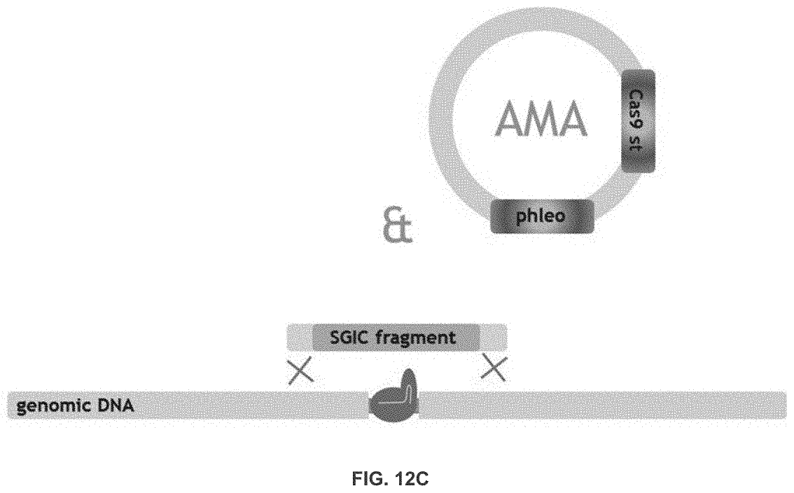

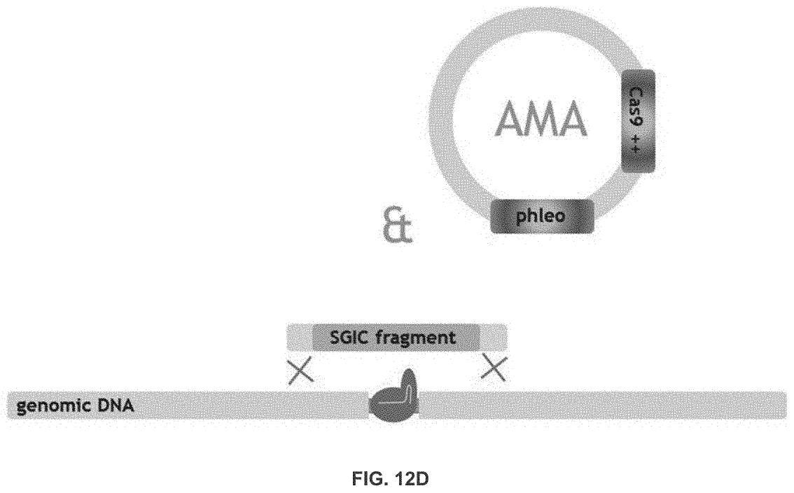

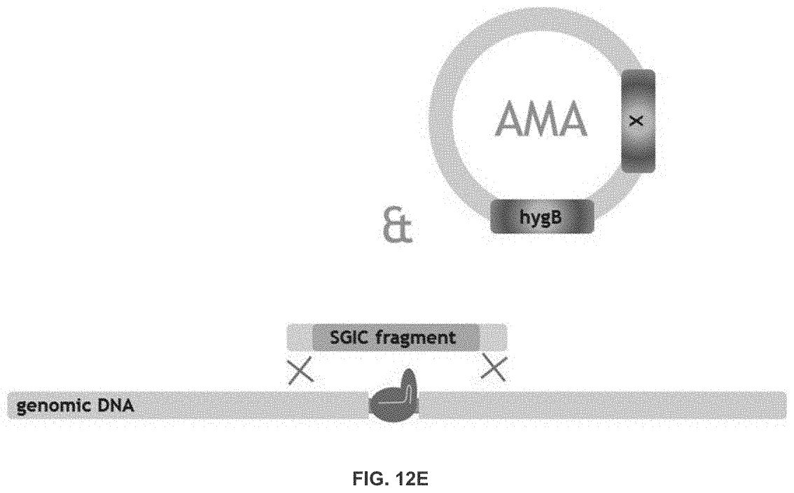

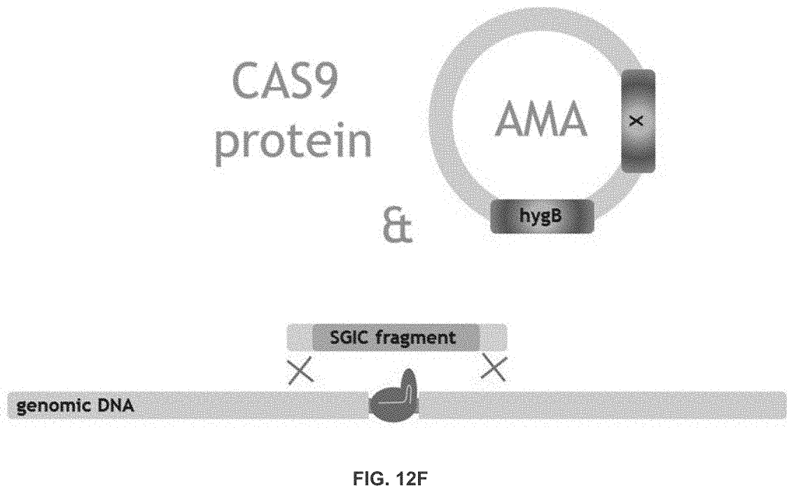

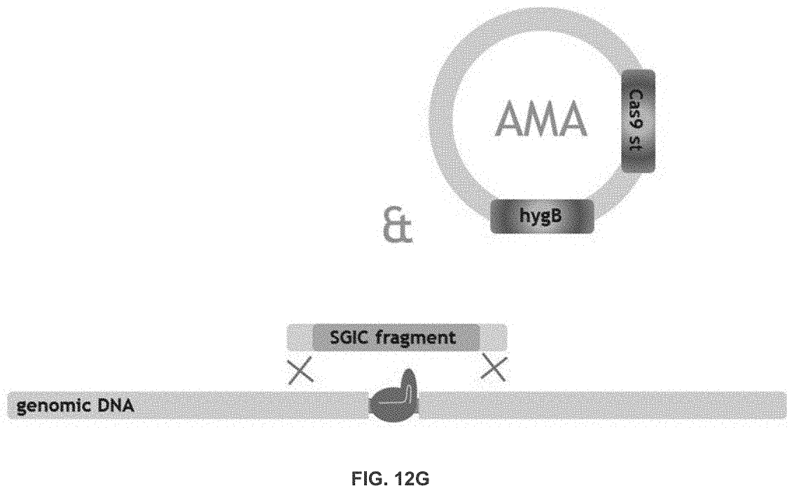

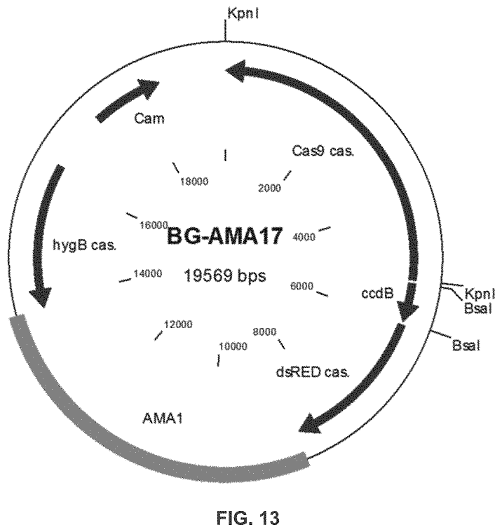

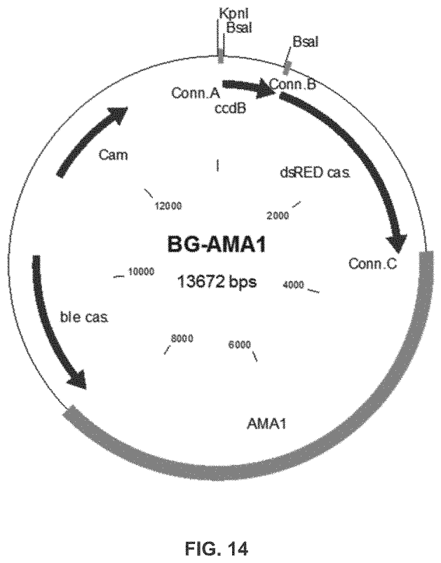

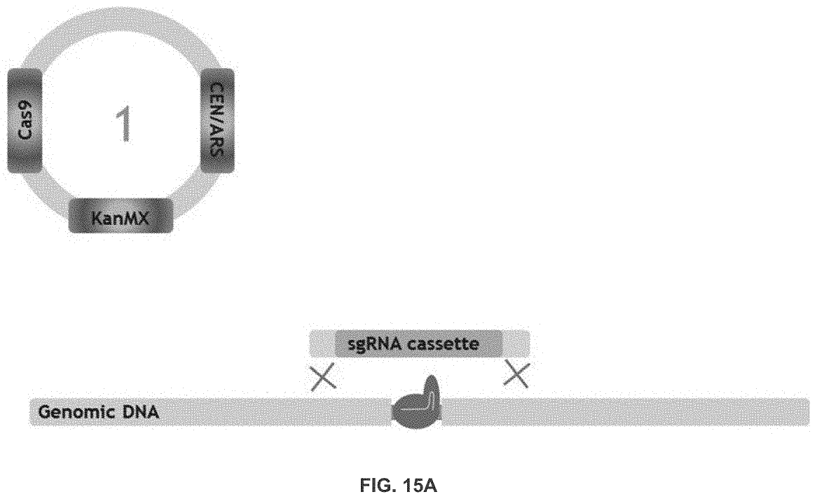

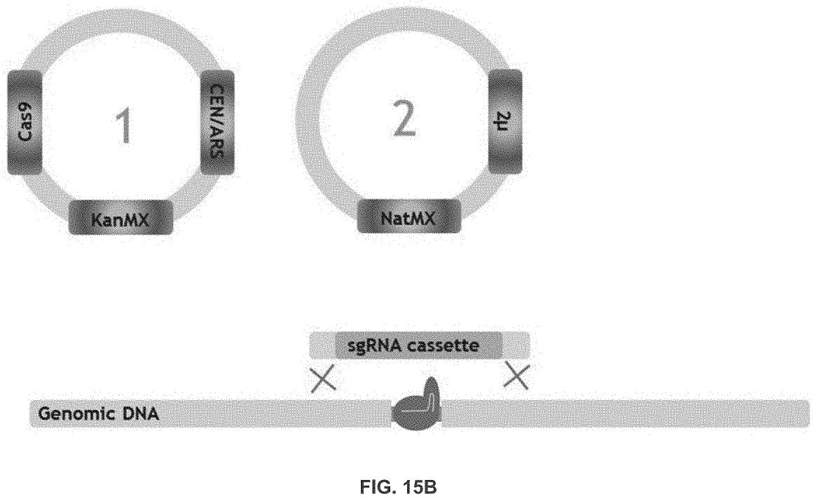

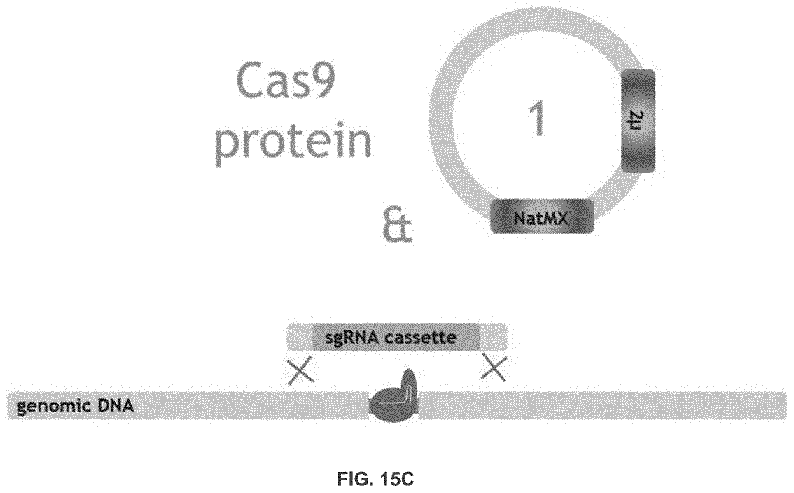

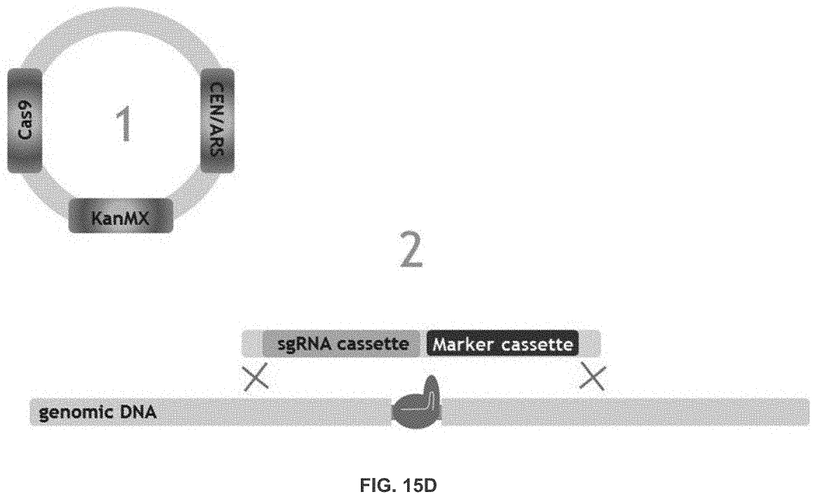

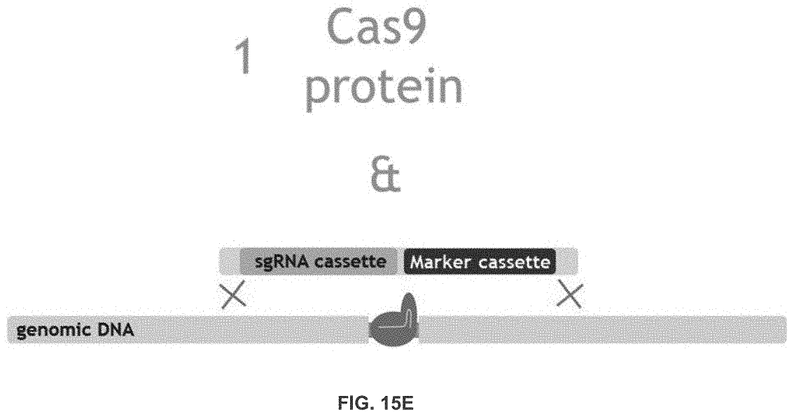

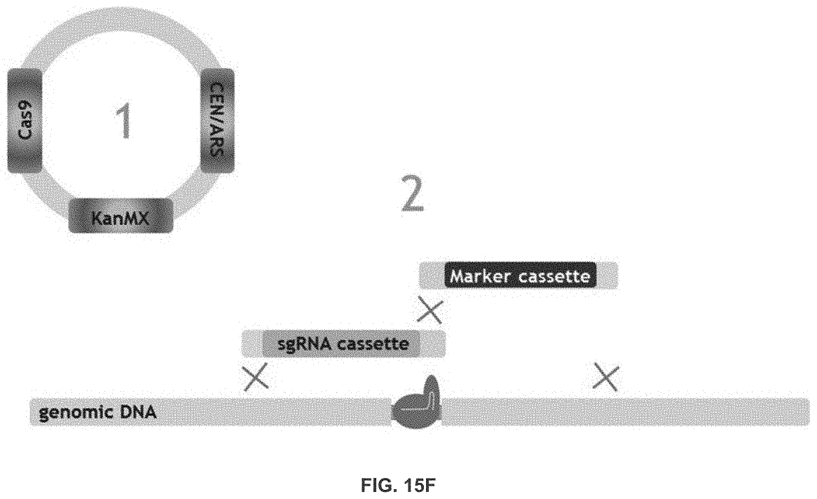

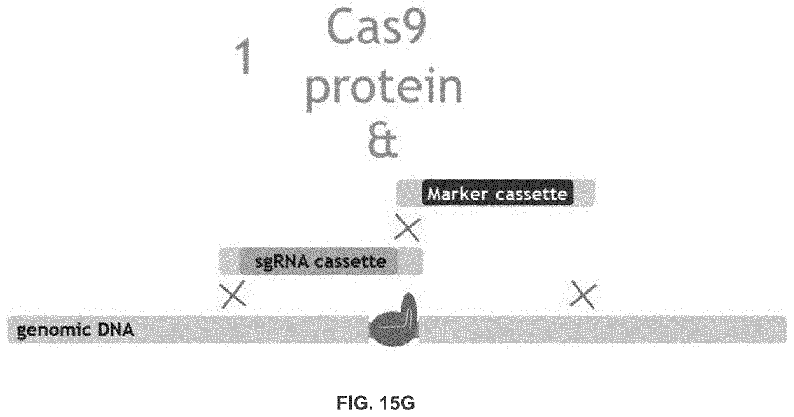

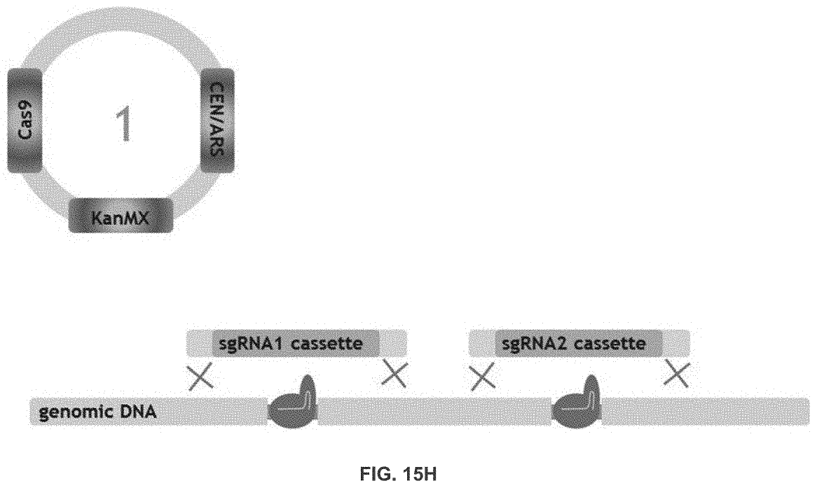

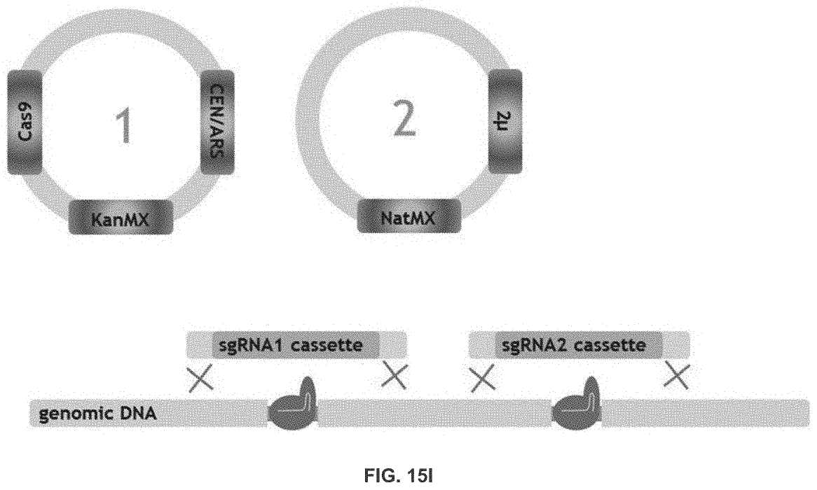

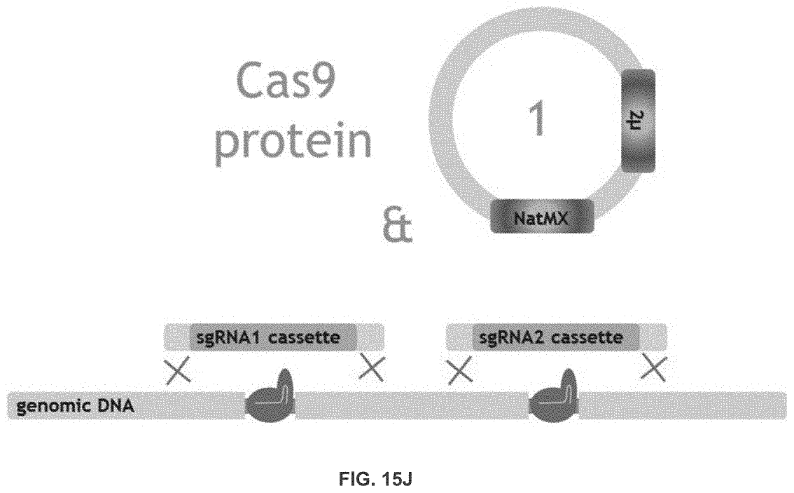

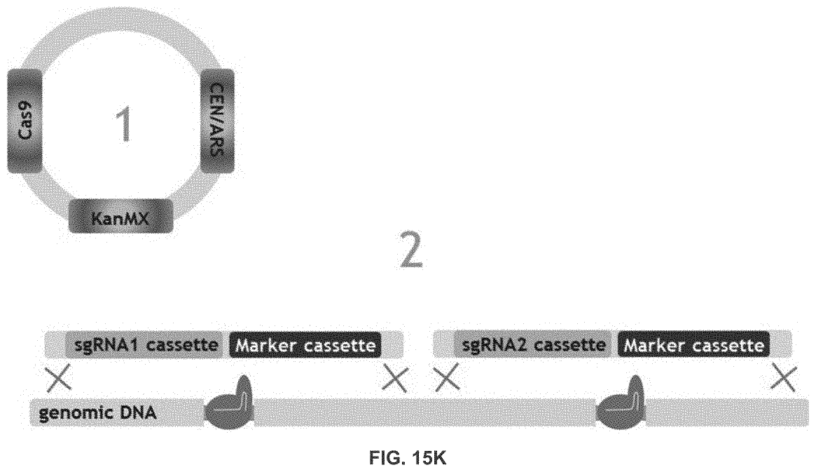

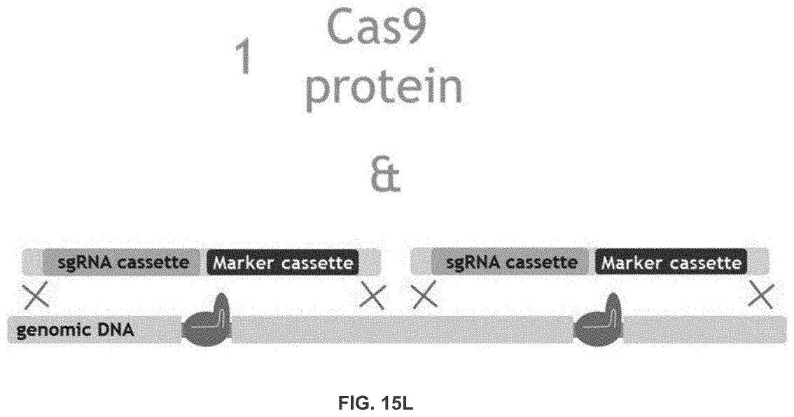

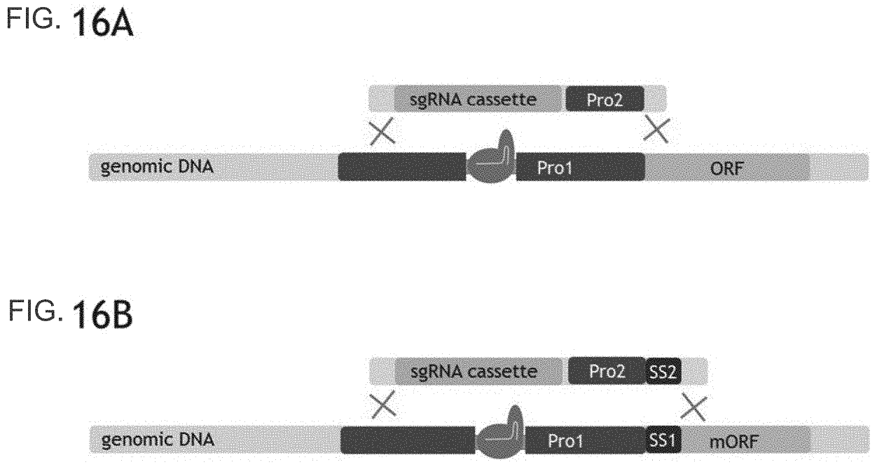

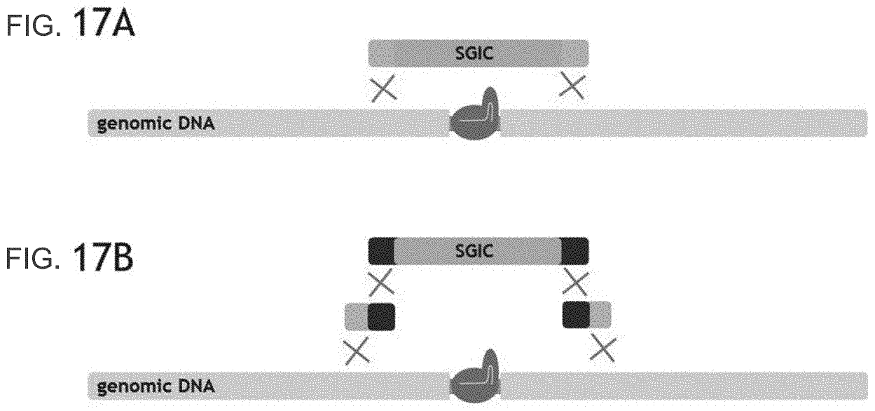

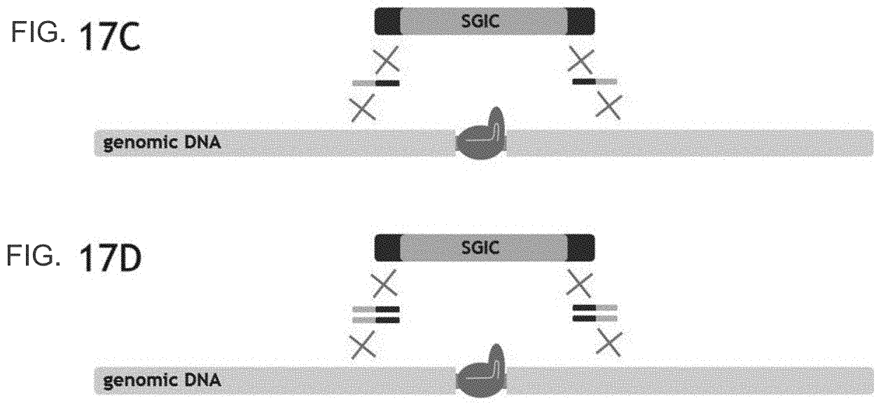

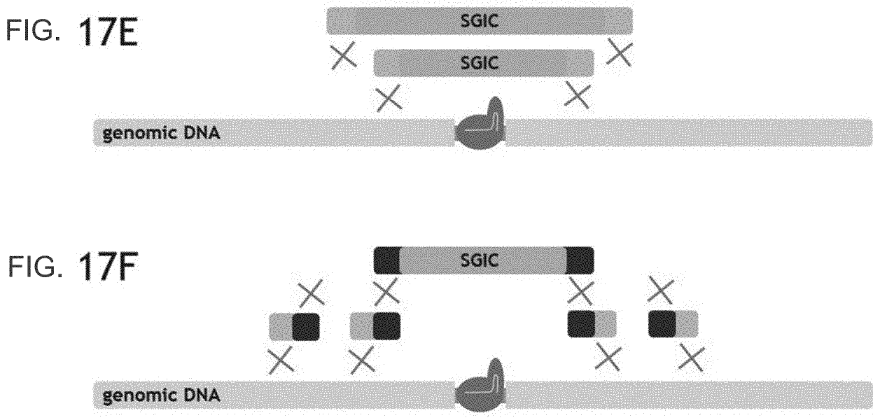

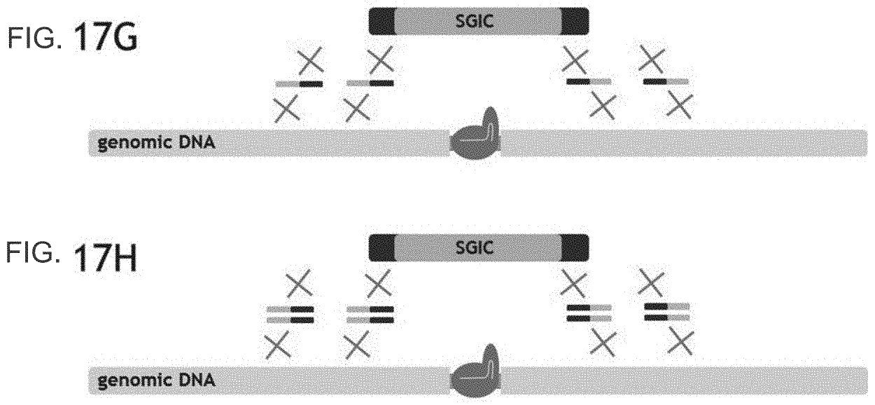

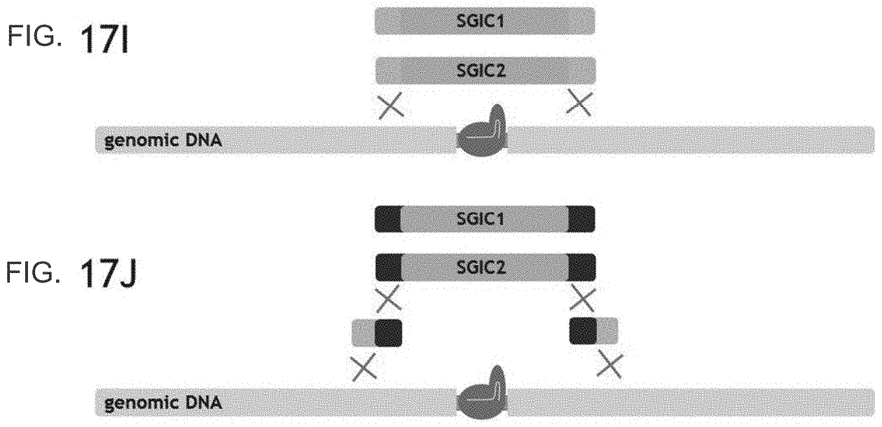

In FIG. 9A, the SGIC contains a sgRNA cassette that targets to the fwnA6 locus and by transient expression and acting together with Cas9 introduces a double-stranded break, indicated by the black triangle. 5′ and 3′ homology flanks are visualized by grey blocks 1 and 2. The SGIC is called ‘SGIC fragment I’ and integrates into the genome by homologous recombination at the fwnA6 locus.

In FIG. 9B, the SGIC contains: (1) a sgRNA cassette that targets to the fwnA6 locus and by transient expression and acting together with Cas9 introduces a double-stranded break, indicated by the black triangle, (2) a Marker cassette, and (3). 5′ and 3′ homology flanks are visualized by the grey blocks 1 and 2. The SGIC called ‘SGIC fragment II A’ or ‘SGIC fragment II B’ and integrates into the genome by homologous recombination at the fwnA6 locus.

In FIG. 9C, the SGIC is a split SGIC comprised of two 2 DNA fragments that upon in vivo assembly in Aspergillus niger form a functional SGIC that contains (1) a sgRNA cassette that targets to the fwnA6 locus and by transient expression and acting together with Cas9 introduces a double-stranded break, indicated by the black triangle, (2) a Marker cassette, and (3) 5′ and 3′ homology flanks are visualized by the grey blocks 1 and 2. The split SGIC fragments used are called ‘SGIC fragment III’ for the left DNA fragment, and ‘SGIC fragment IV A’ or ‘SGIC fragment IV B’ for the right DNA fragment; these fragments recombine in vivo by homology flanks ‘H’ and form a functional SGIC that integrates into the genome by homologous recombination at the fwnA6 locus.

FIG. 10 depicts the map of vector BG-AMA14 used in Example 3.

FIG. 11 depicts the map of vector BG-AMA8 described in WO2016110453A1 and used in Example 3.

FIGS. 12A-12G exemplify various experimental schemes that are applied in Example 3, to show the use of SGIC in Aspergillus niger. FIG. 12A corresponds with row A in Table 10 and Table 11, FIG. 12B corresponds with row B in Table 10 and Table 11, and so on for FIGS. 12C to 12G.

FIG. 13 depicts the map of vector BG-AMA17 used in Example 3.

FIG. 14 depicts the map of vector BG-AMA1 used in Example 3.

FIGS. 15A-15L depict various schemes for the possible and typical use of a Self-Guiding Integration Construct (SGIC) according to the invention comprising a guide-RNA construct capable of expressing a functional guide-RNA that is specific for a target sequence in a target polynucleotide, such as a genome. FIGS. 15A-15L exemplify the use of SGIC in combination with a CRISPR/Cas9 system in Saccharomyces cerevisiae. In practice, Cas9 can be replaced by Cpf1 or another RNA-guided endonuclease, specified markers can be replaced by other suitable markers, and an origin of replication can be replaced by another origin of replication e.g. from a plasmid and/or cassette described elsewhere herein. Within the SGIC, the specified markers can also be replaced by other suitable markers and can even be replaced or supplemented with a functional or non-functional polynucleotide fragment. In case of another RNA-guided endonuclease, an appropriate guide-RNA, sgRNA or crRNA or other suitable RNA sequences that interacts with the RNA-guided endonuclease and targets to a genomic target site can be used instead of the visualized guide-RNA cassette. The visualized guide-RNA cassette can also comprise and encode a partial guide-RNA that together with another externally provided or separately expressed guide-RNA part forms a functional guide-RNA that interacts with the RNA-guided endonuclease and targets the resulting complex to the genomic DNA target. A genomic DNA target site (target polynucleotide) is visualized here by a single box, whereas in practice it could be a collection of multiples, e.g. multiple chromosomes. DNA vectors represented are depicted for application in S. cerevisiae; these can be replaced by suitable vector for other host systems, such as AMA plasmids for filamentous fungi, e.g. Aspergillus niger, as illustrated in examples 2 and 3 in this application. A Cas9 at the genomic DNA target site is in all cases visualized as an egg-shaped blob with in light grey the guide-RNA visualized on it.

FIG. 15A depicts a scheme where Cas9 is being expressed from a first vector 1 with a selectable marker (here KanMX) and the SGIC is introduced in the same transformation. The sgRNA will be transiently expressed from the sgRNA cassette within the SGIC. The linear SGIC will integrate at the genome, facilitated by homology flanks indicated in light grey at the 5′ and 3′ of the SGIC and facilitated by the double stranded break that is generated by Cas9 guided by the sgRNA being expressed from the linear SGIC. During regeneration, selection is made on the marker of vector 1 (here KanMX). Detection of the integrated SGIC can be performed afterwards, e.g. by a suitable PCR reaction, and more specific the integrated sgRNA cassette can be characterized by sequencing the guide-sequence.

FIG. 15B depicts a scheme where Cas9 is being expressed from a first vector 1 with a selectable marker (here KanMX) introduced in the cell in a first transformation, and the SGIC is introduced in a second transformation in the cell together with a vector 2 with a selectable marker (here NatMX). The sgRNA will be transiently expressed from the sgRNA cassette at the SGIC. The linear SGIC will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration of the first transformation round, to enable (pre-)expression of Cas9, selection is made on the marker of vector 1 (here KanMX). During regeneration of the second transformation round using cells that pre-express Cas9, selection is made on the marker of vector 2 (here NatMX), or a double selection is applied for both selectable markers (here KanMX and NatMX) either in a single transformation procedure or two subsequent transformation procedures. Detection of the integrated SGIC can be performed afterwards, e.g. by a suitable PCR reaction, and more specific the integrated sgRNA cassette can be characterized by sequencing the guide-sequence. In an alternative scenario, the first transformation could also be the introduction of a Cas9 expression cassette at the genome of the cell using a suitable transformation construct.

FIG. 15C depicts a scheme where Cas9 is being introduced as a protein together with a SGIC and a vector 1 with a selectable marker (here NatMX)) in the same transformation. The sgRNA will be to transiently expressed from the sgRNA cassette at the SGIC. The linear SGIC will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration, selection is made on the marker of vector 1 (here NatMX). Detection of the integrated SGIC can be performed afterwards, e.g. by a suitable PCR reaction, and more specific the integrated sgRNA cassette can be characterized by sequencing the guide-sequence.

FIG. 15D depicts a scheme where Cas9 is being expressed from a first vector 1 with a selectable marker (here KanMX) introduced in the cell in a first transformation, and the SGIC that contains a selectable marker is introduced in the cell in a second transformation. The sgRNA will be transiently expressed from the sgRNA cassette at the SGIC. The linear SGIC will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration of the first transformation round, selection is made on the marker of vector 1 (here KanMX). During regeneration of the second transformation round, selection is made on the marker of the SGIC, or a double selection is applied for both selectable markers on the vector and SGIC construct. Detection of the integrated SGIC can be performed afterwards, e.g. by a suitable PCR reaction, and more specific the integrated sgRNA cassette can be characterized by sequencing the guide-sequence. Note that the same scheme can be applied in a single transformation round, providing the Cas9 vector (with or without selectable marker and with or without origin of replication, being a linear or a circular construct) together with the SGIC that contains a selectable marker. During regeneration, selection can be made on the selectable marker that is on the SGIC or a double selection for the marker on the Cas9 vector and the selectable marker on the SGIC. In an alternative scenario, the first transformation could also be the introduction of a Cas9 expression cassette at the genome of the cell using a suitable transformation construct.

FIG. 15E depicts a scheme where Cas9 is being introduced as a protein together with a SGIC that contains a sgRNA cassette and a selectable marker, in the cell in the same transformation. The sgRNA will be transiently expressed from the sgRNA cassette at the SGIC. The linear SGIC including a selectable marker will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration, selection is made on the marker on the integrated SGIC at the genomic DNA. Detection of the integrated SGIC can be performed afterwards, e.g. by a suitable PCR reaction, and more specific the integrated sgRNA cassette can be characterized by sequencing the guide-sequence.

FIG. 15F depicts a scheme where Cas9 is being expressed from a first vector 1 with a selectable marker (here KanMX) introduced in the cell in a first transformation. In a second transformation, the SGIC is introduced into the cell as two DNA fragments, that will recombine in-vivo, and after recombination contains a sgRNA cassette and a selectable marker cassette. In this figure the sgRNA cassette is visualized as a left fragment with a 5′ homology flank with the genome, and the right fragment containing the marker cassette with a 3′ homology flank with the genome, whereas both fragments contain a suitable stretch of homologous DNA for in-vivo recombination. In practice, the order and number of DNA fragments can be different, as long as these can assemble into a SGIC with 5′ and 3′homology flanks with the genome. The sgRNA will be transiently expressed from the sgRNA cassette at the SGIC. The linear SGIC will integrate at the genome, facilitated by homology flanks indicated in light grey at the 5′ and 3′ of the sgRNA construct and facilitated by the double stranded break that is generated by Cas9 guided by the sgRNA being expressed from the SGIC. During regeneration of the first transformation round, selection is made on the marker of vector 1 (here KanMX). During regeneration of the second transformation round, selection is made on the marker of the SGIC, or a double selection is applied for both selectable markers on the vector and SGIC. Detection of the integrated SGIC can be performed afterwards, e.g. by a suitable PCR reaction, and more specific the integrated sgRNA cassette can be characterized by sequencing the guide-sequence. Note that the same scheme can be applied in a single transformation round, providing the Cas9 vector (with or without selectable marker and with or without origin of replication, being a linear or a circular construct) together with the SGIC that contains a selectable marker. During regeneration, selection can be made on the selectable marker that is on the SGIC or a double selection for the marker on the Cas9 vector and the selectable marker on the SGIC. In an alternative scenario, the first transformation could also be the introduction of a Cas9 expression cassette at the genome of the cell using a suitable transformation construct.

FIG. 15G depicts a scheme where Cas9 is being introduced into the cell as a protein together with a SGIC as two DNA fragments, that will recombine in-vivo, and after recombination contains a sgRNA cassette and a selectable marker cassette. In this figure the sgRNA cassette is visualized as a left fragment with a 5′ homology flank with the genome, and the right fragment containing the marker cassette with a 3′ homology flank with the genome, whereas both fragments contain a suitable stretch of homologous DNA for in-vivo recombination. In practice, the order and number of DNA fragments can be different, as long as these can assemble into a SGIC with 5′ and 3′homology flanks with the genome. The linear SGIC including a selectable marker will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration, selection is made on the marker on the integrated SGIC at the genomic DNA. Detection of the integrated SGIC can be performed afterwards, e.g. by a suitable PCR reaction, and more specific the integrated sgRNA cassette can be characterized by sequencing the guide-sequence.

FIG. 15H depicts a scheme where Cas9 is being expressed from a first vector 1 with a selectable marker (here KanMX) and two (or more) SGIC are introduced in the same transformation. The two (or more) sgRNA will be transiently expressed from the sgRNA cassette at the SGIC. One (or more) linear SGIC will integrate at the genome, facilitated by homology flanks indicated in light grey at the 5′ and 3′ of the tow (or more) SGIC and facilitated by the two (or more) double stranded breaks that are generated by Cas9 guided by the two (or more) sgRNA being expressed from the two (or more) linear SGIC. During regeneration, selection is made on the marker of vector 1 (here KanMX). Detection of the integrated one (or more) SGIC can be performed afterwards, e.g. by suitable PCR reactions, and more specific the integrated sgRNA cassette can be characterized by sequencing the one (or more) guide-sequences.

FIG. 15I depicts a scheme where Cas9 is being expressed from a first vector 1 with a selectable marker (here KanMX) introduced in the cell in a first transformation, and the two (or more) SGIC are introduced in a second transformation in the cell together with a vector 2 with a selectable marker (here NatMX). The two (or more) sgRNA will be transiently expressed from the sgRNA cassette at the two (or more) SGIC. One (or more) linear SGIC will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration of the first transformation round, selection is made on the marker of vector 1 (here KanMX). During regeneration of the second transformation round, selection is made on the marker of vector 2 (here NatMX), or a double selection is applied for both selectable markers (here KanMX and NatMX). Detection of the one (or more) integrated SGIC can be performed afterwards, e.g. by a suitable PCR reaction, and more specific the one (or more) integrated sgRNA cassette(s) can be characterized by sequencing the guide-sequence. In an alternative scenario, the first transformation could also be the introduction of a Cas9 expression cassette at the genome of the cell using a suitable transformation construct. Alternatively, vectors 1 and 2 and the two (or more) SGIC can be introduced into the cell in a single transformation and selecting on both markers such as KanMX and NatMX during regeneration.

FIG. 15J depicts a scheme where Cas9 is being introduced as a protein together with tow (or more) SGIC and a vector 1 with a selectable marker in the same transformation. The sgRNA will be transiently expressed from the sgRNA cassette at the two (or more) SGIC. One or more SGIC will integrate at the genome, facilitated by homology flanks indicated in light grey at the 5′ and 3′ of the SGIC and facilitated by the double stranded break that is generated by Cas9 guided by the sgRNA being expressed from the SGIC. During regeneration, selection is made on the marker of vector 1 (here NatMX). Detection of the integrated one (or more) SGIC can be performed afterwards, e.g. by a suitable PCR reaction, and more specific the one (or more) integrated sgRNA cassette(s) can be characterized by sequencing the guide-sequence.

FIG. 15K depicts a scheme where Cas9 is being expressed from a first vector 1 with a selectable marker (here KanMX) introduced in the cell in a first transformation, and the two (or more) SGIC that contains a selectable marker are introduced in the cell in a second transformation. The two (or more) sgRNA will be transiently expressed from the two (or more) sgRNA cassettes at the SGIC. The two (or more) linear SGIC will integrate at the genome, facilitated by homology flanks indicated in light grey at the 5′ and 3′ of the two (or more) SGIC and facilitated by the double stranded break that is generated by Cas9 guided by the sgRNA being expressed from the two (or more) SGIC. During regeneration of the first transformation round, selection is made on the marker of vector 1 (here KanMX). During regeneration of the second transformation round, selection is made on the marker of the one (or more) SGIC, or a double (or higher) selection is applied for the selectable marker on the vector and the one or more different selectable markers at the SGIC construct(s). Detection of the integrated two (or more) SGIC can be performed afterwards, e.g. by a suitable PCR reaction, and more specific the one (or more) integrated sgRNA cassette(s) can be characterized by sequencing the guide-sequence. Note that the same scheme can be applied in a single transformation round, providing the Cas9 vector (with or without selectable marker and with or without origin of replication, being a linear or a circular construct) together with the SGIC that contains a selectable marker. During regeneration, selection can be made on the selectable marker that is on the SGIC or a double selection for the marker on the Cas9 vector and the selectable marker on the SGIC. In an alternative scenario, the first transformation could also be the introduction of a Cas9 expression cassette at the genome of the cell using a suitable transformation construct.

FIG. 15L depicts a scheme where Cas9 is introduced as a protein together with two (or more) SGIC that contains a sgRNA cassette and a selectable marker (where both SGIC may contain the same selectable marker or a different one), in the cell in the same transformation. The sgRNA will be transiently expressed from the sgRNA cassette at the two (or more) SGIC. The one (or more) linear SGIC including a selectable marker will integrate at the genome, facilitated by homology flanks indicated in light grey at the 5′ and 3′ of the two (or more) SGIC and facilitated by the double stranded break that is generated by Cas9 guided by the sgRNA being expressed from the two (or more) SGIC. During regeneration, selection is made on the marker on the one (or more) integrated SGIC at the genomic DNA. Detection of the one (or more) integrated SGIC can be performed afterwards, e.g. by a suitable PCR reaction, and more specific the one (or more) integrated sgRNA cassette(s) can be characterized by sequencing the guide-sequence.

FIGS. 16A-16B depict examples of SGIC constructs that can be used to replace or insert a control sequence in the genomic DNA. The SGIC is applied in combination with a RNA guided endonuclease, indicated as the egg-shaped blob at the genomic DNA box visualization.

FIG. 16A depicts the use of a SGIC construct to replace (or insert) a promoter (Pro1), or a part thereof by a new promoter DNA sequence (Pro2). The 5′ and 3′ homology flanks at the SGIC determine what part of the genomic DNA will be replaced by the SGIC insert. ORF here indicates the open reading frame of a gene. In a preferred situation, the homology flanks are chosen in such a way that in vivo recombination with the genomic DNA (facilitated by a single or double stranded break) leads to a functional expression of the ORF at the genome, where the Pro1 (or a part thereof) is replaced by a Pro2 that is e.g. weaker or stronger, inducible or has another characteristic than Pro1. In another situation, multiple SGIC (with the same or with different sgRNA cassettes, with same or different homology flanks) can be provided in a same transformation to generate a library of replacements of Pro1. In another situation, multiple SGIC (with the same or with different sgRNA cassettes, with same or different homology flanks) can be provided in a single transformation experiment to generate a library targeting different ORFs at the genome, and generating one or more promoter replacements at the genome of a cell. This example visualization is not limited to Cas9, and should be seen as an illustration showing the principle of promoter replacement that can also be applied with other RNA guided endonucleases, e.g. Cpf1 with the corresponding RNA expression cassettes at a applied SGIC.

FIG. 16B depicts the replacement of a promoter (Pro1) and a signal sequence (SS1), e.g., a secretion signal, prepro sequence etc. with another Pro2 and signal sequence SS2. In both cases FIGS. 16A and 16B, additional elements like a suitable marker cassette can be part of the SGIC. In the figure mORF is an abbreviation for ORF encoding for the mature protein, meaning without the signal sequence.

FIGS. 17A-17J depict various examples of use of the SGIC according to the invention. It should be noted that the use as depicted in FIGS. 17A-17J can conveniently be combined with the us as depicted in FIGS. 15A-15L and 16A-16B. The SGIC is applied in combination with a RNA guided endonuclease, indicated as the egg-shaped blob at the genomic DNA box visualization.

FIG. 17A depicts the use of a SGIC with 5′ and 3′ homology flanks for integration at the genomic DNA, as visualized by the grey blocks.

FIG. 17B depicts the use of a SGIC with 5′ and 3′ homology flanks with separate double-stranded DNA flanks (visualized by the black boxes on SGIC and the separate flanks) that by itself have 5′ or 3′ homology for integration at the genomic DNA, as visualized by the grey blocks. By in-vivo homologous recombination, the SGIC will integrate at the genome.

FIG. 17C depicts the use of a SGIC with 5′ and 3′ homology flanks with separate single-stranded ODN flanks (visualized by the black boxes on SGIC and the separate ssODNs) that by itself have 5′ or 3′ homology for integration at the genomic DNA, as visualized by the grey blocks. By in-vivo homologous recombination, the SGIC will integrate at the genome.

FIG. 17D depicts the use of a SGIC with 5′ and 3′ homology flanks with 2 sets of separate complementary single-stranded ODN flanks (visualized by the black boxes on SGIC and the separate ssODNs) that by itself have 5′ or 3′ homology for integration at the genomic DNA, as visualized by the grey blocks. By in-vivo homologous recombination, the SGIC will integrate at the genome.

FIG. 17E depicts the use of a SGIC in a similar way as FIG. 17A. Here, two or more SGIC are provided with 5′ and 3′ homology flanks for integration at the genomic DNA, as visualized by the grey blocks. By providing SGIC with different homology flanks with the genomic DNA in one transformation, a library of cells with SGIC integrated at different positions (determined by the homology flanks of the SGIC's applied) on the genomic DNA will result.

FIG. 17F depicts the use of a SGIC in a similar way as FIG. 17B. Here, three or more separate double-stranded DNA flanks (visualized by the black boxes on SGIC and the separate flanks) that by itself have 5′ or 3′ homology for integration at the genomic DNA, as visualized by the grey blocks. By providing SGIC with different homology flanks with the genomic DNA in one transformation, a library of cells with SGIC integrated at different positions (determined by the homology flanks of the double-stranded DNA flanks applied) on the genomic DNA will result.

FIG. 17G depicts the use of a SGIC in a similar way as FIG. 17C. Here, three or more separate single-stranded ODN flanks (visualized by the black boxes on SGIC and the separate ssODNs) that by itself have 5′ or 3′ homology for integration at the genomic DNA, as visualized by the grey blocks. By providing SGIC with different homology flanks with the genomic DNA in one transformation, a library of cells with SGIC integrated at different positions (determined by the homology flanks of the ssODN flanks applied) on the genomic DNA will result.

FIG. 17H depicts the use of a SGIC in a similar way as FIG. 17D. Here, three or more sets of complementary single-stranded ODN flanks (visualized by the black boxes on SGIC and the separate ssODNs) that by itself have 5′ or 3′ homology for integration at the genomic DNA, as visualized by the grey blocks. By providing SGIC with different homology flanks with the genomic DNA in one transformation, a library of cells with SGIC integrated at different positions (determined by the homology flanks of the sets of complementary ssODN flanks applied) on the genomic DNA will result.

FIG. 17I depicts the use of a SGIC in a similar way as FIG. 17A. Here, two or more SGIC are provided with 5′ and 3′ homology flanks for integration at the genomic DNA, as visualized by the grey blocks. By providing SGIC1, SGIC2 (or more till SGICn) with different DNA elements, a library of cells with SGIC integrated at the same positions on the genomic DNA will result. Examples (but not limited to these) of use can be that the SGIC1, SGIC2 till SGICn differ in sgRNA guide, targeting a different cleavage locus, or for example contain a different DNA promoter element to be introduced at the genome to replace an existing promoter). By providing SGIC with different DNA elements, a library of cells with SGIC1, SGIC2 (or more) integrated at different positions on the genomic DNA will result.

FIG. 17J depicts the use of a SGIC in a similar way as FIG. 17B. Here, two or more SGIC are provided with 5′ and 3′ homology flanks with separate double-stranded DNA flanks (visualized by the black boxes on SGIC and the separate flanks) that by itself have 5′ or 3′ homology for integration at the genomic DNA, as visualized by the grey blocks. By providing SGIC1, SGIC2 (or more till SGICn) with different DNA elements, a library of cells with SGIC integrated at the same positions on the genomic DNA will result. Examples (but not limited to these) of use can be that the SGIC1, SGIC2 till SGICn differ in sgRNA guide, targeting a different cleavage locus, or for example contain a different DNA promoter element to be introduced at the genome to replace an existing promoter). By providing SGIC with different DNA elements, a library of cells with SGIC1, SGIC2 (or more) integrated at different positions on the genomic DNA will result.

### DESCRIPTION OF THE SEQUENCES

SEQ ID NO: 1 sets out the nucleotide sequence of Cas9 including a C-terminal SV40 nuclear localization signal codon pair optimized for expression in Saccharomyces cerevisiae. The sequence includes the Kill promoter (promoter of KLLA0F20031g) from Kluyveromyces lactis and the GND2 terminator sequence from Saccharomyces cerevisiae.

SEQ ID NO: 2 sets out the nucleotide sequence of vector pCSN061.

SEQ ID NO: 3 sets out the nucleotide sequence of vector pRN1120.

SEQ ID NO: 4 sets out the nucleotide sequence of the gBlock of the guide-RNA expression cassette to target Cas9 to the INT1 locus.

SEQ ID NO: 5 sets out the nucleotide sequence of the gBlock of the guide-RNA expression cassette to target Cas9 to the INT59 locus.

SEQ ID NO: 6 sets out the nucleotide sequence of the gBlock of the guide-RNA expression cassette to target Cas9 to the YPRCtau3 locus.

SEQ ID NO: 7 sets out the nucleotide sequence of the guide sequence (genomic target sequence) of the INT1 integration site.

SEQ ID NO: 8 sets out the nucleotide sequence of the guide sequence (genomic target sequence) of the INT59 integration site.

SEQ ID NO: 9 sets out the nucleotide sequence of the guide sequence (genomic target sequence) of the YPRCtau3 integration site.

SEQ ID NO: 10 sets out the nucleotide sequence of the FW primer to obtain INT1 SGIC DNA sequence for integration, 0 kbp deletion.

SEQ ID NO: 11 sets out the nucleotide sequence of REV primer to obtain INT1 SGIC DNA sequence for integration, 0 kbp deletion.

SEQ ID NO: 12 sets out the nucleotide sequence of the FW primer to obtain INT1 SGIC DNA sequence for integration, 1 kbp deletion.

SEQ ID NO: 13 sets out the nucleotide sequence of REV primer to obtain INT1 SGIC DNA sequence for integration, 1 kbp deletion.

SEQ ID NO: 14 sets out the nucleotide sequence of the FW primer to obtain INT59 SGIC DNA sequence for integration, 0 kbp deletion.

SEQ ID NO: 15 sets out the nucleotide sequence of REV primer to obtain INT59 SGIC DNA sequence for integration, 0 kbp deletion.

SEQ ID NO: 16 sets out the nucleotide sequence of the FW primer to obtain INT59 SGIC DNA sequence for integration, 1 kbp deletion.

SEQ ID NO: 17 sets out the nucleotide sequence of REV primer to obtain INT59 SGIC DNA sequence for integration, 1 kbp deletion.

SEQ ID NO: 18 sets out the nucleotide sequence of the FW primer to obtain YPRCtau3 SGIC DNA sequence for integration, 0 kbp deletion.

SEQ ID NO: 19 sets out the nucleotide sequence of REV primer to obtain YPRCtau3 SGIC DNA sequence for integration, 0 kbp deletion.

SEQ ID NO: 20 sets out the nucleotide sequence of the FW primer to obtain YPRCtau3 SGIC DNA sequence for integration, 1 kbp deletion.

SEQ ID NO: 21 sets out the nucleotide sequence of REV primer to obtain YPRCtau3 SGIC DNA sequence for integration, 1 kbp deletion.

SEQ ID NO: 22 sets out the nucleotide sequence of INT1 SGIC DNA sequence for integration, 0 kbp deletion.

SEQ ID NO: 23 sets out the nucleotide sequence of INT1 SGIC DNA sequence for integration, 1 kbp deletion.

SEQ ID NO: 24 sets out the nucleotide sequence of INT59 SGIC DNA sequence for integration, 0 kbp deletion.

SEQ ID NO: 25 sets out the nucleotide sequence of INT59 SGIC DNA sequence for integration, 1 kbp deletion.

SEQ ID NO: 26 sets out the nucleotide sequence of YPRCtau3 SGIC DNA sequence for integration, 0 kbp deletion.

SEQ ID NO: 27 sets out the nucleotide sequence of YPRCtau3 SGIC DNA sequence for integration, 1 kbp deletion.

SEQ ID NO: 28 sets out the nucleotide sequence of the FW primer annealing to SNR52p to obtain SGIC DNA sequence for integration without genomic flanking regions attached.

SEQ ID NO: 29 sets out the nucleotide sequence of the REV primer annealing to SUP4 3′ flanking region to obtain SGIC DNA sequence for integration without genomic flanking regions attached.

SEQ ID NO: 30 sets out the nucleotide sequence of INT1 SGIC DNA without genomic flanking regions attached on either side.

SEQ ID NO: 31 sets out the nucleotide sequence of INT59 SGIC DNA without genomic flanking regions attached on either side.

SEQ ID NO: 32 sets out the nucleotide sequence of YPRCtau3 SGIC DNA without genomic flanking regions attached on either side.

SEQ ID NO: 33 sets out the nucleotide sequence of the FW primer to confirm integration of the SGIC DNA in the INT1 locus, 0 kbp deletion.

SEQ ID NO: 34 sets out the nucleotide sequence of the REV primer to confirm integration of the SGIC DNA in the INT1 locus, 0 kbp deletion.

SEQ ID NO: 35 sets out the nucleotide sequence of the FW primer to confirm integration of the SGIC DNA in the INT1 locus, 1 kbp deletion.

SEQ ID NO: 36 sets out the nucleotide sequence of the REV primer to confirm integration of the SGIC DNA in the INT1 locus, 1 kbp deletion.

SEQ ID NO: 37 sets out the nucleotide sequence of the FW primer to confirm integration of the SGIC DNA in the INT59 locus, 0 kbp deletion.

SEQ ID NO: 38 sets out the nucleotide sequence of the REV primer to confirm integration of the SGIC DNA in the INT59 locus, 0 kbp deletion.

SEQ ID NO: 39 sets out the nucleotide sequence of the FW primer to confirm integration of the SGIC DNA in the INT59 locus, 1 kbp deletion.

SEQ ID NO: 40 sets out the nucleotide sequence of the REV primer to confirm integration of the SGIC DNA in the INT59 locus, 1 kbp deletion.

SEQ ID NO: 41 sets out the nucleotide sequence of the FW primer to confirm integration of the SGIC DNA in the YPRCtau3 locus, 0 kbp deletion.

SEQ ID NO: 42 sets out the nucleotide sequence of the REV primer to confirm integration of the SGIC DNA in the YPRCtau3 locus, 0 bp deletion.

SEQ ID NO: 43 sets out the nucleotide sequence of the FW primer to confirm integration of the SGIC DNA in the YPRCtau3 locus, 1 kbp deletion.

SEQ ID NO: 44 sets out the nucleotide sequence of the REV primer to confirm integration of the SGIC DNA in the YPRCtau3 locus, 1 kbp deletion.

SEQ ID NO: 45 sets out the nucleotide sequence of the FW primer annealing to SNR52p to obtain INT1 SGIC DNA sequence with 50 bp connector sequence at the 5′ end.

SEQ ID NO: 46 sets out the nucleotide sequence of the REV primer annealing to SUP4 to obtain INT1 SGIC DNA sequence with 50 bp connector sequence at the 3′ end.

SEQ ID NO: 47 sets out the nucleotide sequence of the SGIC DNA with connector sequences attached to the 5′ and 3′ ends.

SEQ ID NO: 48 sets out the nucleotide sequence of the REV primer annealing to SNR52p to obtain the 5′ split SGIC DNA sequence targeting INT1.

SEQ ID NO: 49 sets out the nucleotide sequence of the FW primer annealing to the guide-RNA to obtain the 3′ split SGIC DNA sequence targeting INT1.

SEQ ID NO: 50 sets out the nucleotide sequence of the FW primer annealing to the 5′ connector of SGIC DNA fragment to attach genomic DNA sequence for integration on INT1.

SEQ ID NO: 51 sets out the nucleotide sequence of the RV primer annealing to the 3′ connector of SGIC DNA fragment to attach genomic DNA sequence for integration on INT1.

SEQ ID NO: 52 sets out the nucleotide sequence of the SGIC DNA with 50 bp genomic DNA sequences attached on both the 5′ and 3′ end for integration on INT1.

SEQ ID NO: 53 sets out the nucleotide sequence of the 5′ fragment of the split SGIC DNA with 50 bp homology to the 3′ split SGIC DNA for assembly.

SEQ ID NO: 54 sets out the nucleotide sequence of the 3′ fragment of the split SGIC DNA with 50 bp homology to the 5′ split SGIC DNA for assembly.

SEQ ID NO: 55 sets out the nucleotide sequence of ssODN 5′ flank 1 kbp upper strand sequence.

SEQ ID NO: 56 sets out the nucleotide sequence of ssODN 5′ flank 1 kbp lower strand sequence.

SEQ ID NO: 57 sets out the nucleotide sequence of ssODN 3′ flank 1 kbp upper strand sequence.

SEQ ID NO: 58 sets out the nucleotide sequence of ssODN 3′ flank 1 kbp lower strand sequence.

SEQ ID NO: 59 sets out the nucleotide sequence of the connector sequence on the 5′ end of the SGIC DNA.

SEQ ID NO: 60 sets out the nucleotide sequence of the connector sequence on the 3′ end of the SGIC DNA.

SEQ ID NO: 61 sets out the nucleotide sequence of forward PCR primer SGIC DNA part 5′ fwnA flank-sgRNA-3′ conH.

SEQ ID NO: 62 sets out the nucleotide sequence of reverse PCR primer SGIC DNA part 5′ fwnA flank-sgRNA-3′ conH.

SEQ ID NO: 63 sets out the nucleotide sequence of forward PCR primer SGIC DNA hygB or phleo marker-3′ fnwA flank.

SEQ ID NO: 64 sets out the nucleotide sequence of reverse PCR primer SGIC DNA hygB or phleo marker-3′ fnwA flank.

SEQ ID NO: 65 sets out the nucleotide sequence of BG-AMA5 AMA phleo/Cas9 st.

SEQ ID NO: 66 sets out the nucleotide sequence of BG-AMA9 AMA hygB/Cas9 st./sgRNA cassette.

SEQ ID NO: 67 sets out the nucleotide sequence of the TOPO Zero Blunt cloning vector.

SEQ ID NO: 68 sets out the nucleotide sequence of backbone vector AB.

SEQ ID NO: 69 sets out the nucleotide sequence of vector SGIC DNA hygB.

SEQ ID NO: 70 sets out the nucleotide sequence of vector SGIC DNA phleo.

SEQ ID NO: 71 sets out the nucleotide sequence of reverse PCR primer SGIC fragment I.

SEQ ID NO: 72 sets out the nucleotide sequence of forward PCR primer SGIC fragment II and III.

SEQ ID NO: 73 sets out the nucleotide sequence of reverse PCR primer SGIC fragment II and IV.

SEQ ID NO: 74 sets out the nucleotide sequence of reverse PCR primer SGIC fragment III.

SEQ ID NO: 75 sets out the nucleotide sequence of forward PCR primer SGIC fragment IV.

SEQ ID NO: 76 sets out the nucleotide sequence of TOPO SGIC DNA sgRNA fwnA.

SEQ ID NO: 77 sets out the nucleotide sequence of TOPO SGIC hygB.

SEQ ID NO: 78 sets out the nucleotide sequence of TOPO SGIC phleo.

SEQ ID NO: 79 sets out the nucleotide sequence of forward PCR primer Cas9 with KpnI-flank.

SEQ ID NO: 80 sets out the nucleotide sequence of reverse PCR primer Cas9 with KpnI-flank.

SEQ ID NO: 81 sets out the nucleotide sequence of BG-AMA8 AMA hygB/no Cas9 expression cassette.

SEQ ID NO: 82 sets out the nucleotide sequence of BG-AMA14 AMA phleo/Cas9++.

SEQ ID NO: 83 sets out the nucleotide sequence of BG-AMA17 AMA hygB/Cas9 st.

SEQ ID NO: 84 sets out the nucleotide sequence of BG-AMA1 AMA phleo/no Cas9 expression cassette.

SEQ ID NO: 85 sets out the nucleotide sequence of SGIC DNA fragment I (see Table 9).

SEQ ID NO: 86 sets out the nucleotide sequence of SGIC DNA fragment II A (see Table 9).

SEQ ID NO: 87 sets out the nucleotide sequence of SGIC DNA fragment II B (see Table 9).

SEQ ID NO: 88 sets out the nucleotide sequence of SGIC DNA fragment III (see Table 9).

SEQ ID NO: 89 sets out the nucleotide sequence of SGIC DNA fragment IV A (see Table 9).

SEQ ID NO: 90 sets out the nucleotide sequence of SGIC DNA fragment IV B (see Table 9).

SEQ ID NO: 91 sets out the nucleotide sequence of the gBlock that contains the sgRNA expression cassette to target ORF1; i.e. ORF1_SGIC DNA before the genomic flanking regions are added to either 5′ and 3′ end.

SEQ ID NO: 92 sets out the nucleotide sequence of the gBlock that contains the sgRNA expression cassette to target ORF2; i.e. ORF2_SGIC DNA before the genomic flanking regions are added to either 5′ and 3′ end.

SEQ ID NO: 93 sets out the nucleotide sequence of the gBlock that contains the sgRNA expression cassette to target ORF3; i.e. ORF3_SGIC DNA before the genomic flanking regions are added to either 5′ and 3′ end.

SEQ ID NO: 94 sets out the nucleotide sequence of the guide sequence (genomic target sequence) of ORF1.

SEQ ID NO: 95 sets out the nucleotide sequence of the guide sequence (genomic target sequence) of ORF2.

SEQ ID NO: 96 sets out the nucleotide sequence of the guide sequence (genomic target sequence) of ORF3.

SEQ ID NO: 97 sets out the nucleotide sequence of the forward primer to obtain ORF1_SGIC DNA sequence for integration.

SEQ ID NO: 98 sets out the nucleotide sequence of the reverse primer to obtain ORF1_SGIC DNA sequence for integration.

SEQ ID NO: 99 sets out the nucleotide sequence of the forward primer to obtain ORF2_SGIC DNA sequence for integration.

SEQ ID NO: 100 sets out the nucleotide sequence of the reverse primer to obtain ORF2_SGIC DNA sequence for integration.

SEQ ID NO: 101 sets out the nucleotide sequence of the forward primer to obtain ORF3 SGIC_DNA sequence for integration.

SEQ ID NO: 102 sets out the nucleotide sequence of the reverse primer to obtain ORF3_SGIC DNA sequence for integration.

SEQ ID NO: 103 sets out the nucleotide sequence of ORF1_SGIC DNA with genomic flanking regions attached at both the 5′ and 3′ end for integration.

SEQ ID NO: 104 sets out the nucleotide sequence of ORF2_SGIC DNA with genomic flanking regions attached at both the 5′ and 3′ end for integration.

SEQ ID NO: 105 sets out the nucleotide sequence of ORF3_SGIC DNA with genomic flanking regions attached at both the 5′ and 3′ end for integration.

SEQ ID NO: 106 sets out the nucleotide sequence of forward primer to confirm knock out of ORF1 by integration of ORF1_SGIC DNA.

SEQ ID NO: 107 sets out the nucleotide sequence of reverse primer to confirm knock out of ORF1 by integration of ORF1_SGIC DNA.

SEQ ID NO: 108 sets out the nucleotide sequence of forward primer to confirm knock out of ORF2 by integration of ORF2_SGIC DNA.

SEQ ID NO: 109 sets out the nucleotide sequence of reverse primer to confirm knock out of ORF2 by integration of ORF2_SGIC DNA.

SEQ ID NO: 110 sets out the nucleotide sequence of forward primer to confirm knock out of ORF3 by integration of ORF3_SGIC DNA.

SEQ ID NO: 111 sets out the nucleotide sequence of reverse primer to confirm knock out of ORF3 by integration of ORF3_SGIC DNA.

### DETAILED DESCRIPTION

The inventors have found that a self-guiding integration construct comprising a guide-RNA construct capable of expressing a functional guide-RNA that is specific for a target sequence in a target polynucleotide, wherein said guide-RNA construct is flanked by a 5′-polynucleotide and a 3′-polynucleotide that have sequence identity with sequences flanking the target sequence in the target polynucleotide, said construct optionally further comprising an additional functional or non-functional polynucleotide element, provides a great improvement. In this system, the guide-RNA is initially expressed from the self-guiding integration construct. The expressed guide-RNA facilitates induction of a break into the target genome at the target sequence and subsequently the self-guiding integration construct integrates into the target genome. This system can, e.g., conveniently be used using a library of self-guiding integration constructs where distinct additional functional or non-functional polynucleotide elements are present on the constructs which are linked to the guide-RNA's. The SGIC as provided herein can be viewed as a donor polynucleotide in the sense as known in the art of e.g. CRISPR/Cas gene editing, which contains a guide-RNA expression cassette.

Using polynucleotide-guided nuclease/editing systems such as the CRISPR/Cas9 system, there is the possibility to develop gene drives capable of autonomously spreading genomic alterations by organisms via sexual replication, e.g. explained by DiCarlo et al., 2015. Neither the inventors, nor the applicant has intended, intends or will intend to create such gene drives or likewise autonomous gene editing tools (also known as mutagenic chain reaction or active genetics).

In a first aspect, there is provided for a self-guiding integration construct (SGIC) comprising:
a guide-RNA expression cassette, and
an additional polynucleotide element,
   wherein said guide-RNA expression cassette is capable of expressing a functional guide-RNA, or a part thereof, that is specific for a target sequence in a target genome, wherein the part of the self-guiding integration construct comprising said guide-RNA expression cassette and said donor polynucleotide part is flanked at its 5′-terminus by a first polynucleotide and at its 3′-terminus by a second polynucleotide, and wherein said first and second polynucleotide have sequence identity with sequences flanking the target sequence in the target genome.

In addition, there is provided for a self-guiding integration construct comprising:
a guide-RNA expression cassette, and optionally,
an additional polynucleotide element,
   wherein said guide-RNA expression cassette is capable of expressing a functional guide-RNA, or a part thereof, that is specific for a target sequence in a target genome, wherein the part of the self-guiding integration construct comprising said guide-RNA expression cassette and said optional additional polynucleotide element is flanked at its 5′-terminus by a first polynucleotide and at its 3′-terminus by a second polynucleotide, wherein said first and second polynucleotide have sequence identity with sequences flanking the target sequence in the target genome, and wherein the functional guide-RNA, or the part thereof, is encoded by a polynucleotide on the guide-RNA expression cassette and said polynucleotide is operably linked to an RNA polymerase II promoter, to an RNA polymerase III promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter.

In addition, there is provided for a self-guiding integration construct comprising:

two or more polynucleotides capable of recombining with each other to yield a guide-RNA expression cassette optionally comprising an additional polynucleotide element,
wherein said guide-RNA expression cassette is capable of expressing a functional guide-RNA, or a part thereof, wherein said functional guide-RNA or part thereof is specific for a target sequence in a target genome, wherein the part of the self-guiding integration construct comprising said guide-RNA expression cassette and optionally said additional polynucleotide element is flanked at its 5′-terminus by a first polynucleotide and at its 3′-terminus by a second polynucleotide, and wherein said first and second polynucleotide have sequence identity with sequences flanking the target sequence in the target genome. A non-limiting example of such self-guiding integration construct is depicted in FIGS. 15A-15L.

In addition, there is provided for a composition comprising two or more polynucleotide members, wherein these members have sequence identity with each other which allows them to recombine in vivo, such as in a host cell, to yield a single self-guiding integration construct comprising:
a guide-RNA expression cassette, and
an additional polynucleotide element,
   wherein said guide-RNA expression cassette is capable of expressing a functional guide-RNA, or a part thereof, that is specific for a target sequence in a target genome, wherein the part of the self-guiding integration construct comprising said guide-RNA expression cassette and said additional polynucleotide element is flanked at its 5′-terminus by a first polynucleotide and at its 3′-terminus by a second polynucleotide, and wherein said first and second polynucleotide have sequence identity with sequences flanking the target sequence in the target genome. A non-limiting example of such composition as disclosed herein yielding a self-guiding integration construct as disclosed herein is depicted in FIGS. 15A-15L.

In addition, there is provided for a composition according to the invention comprising two or more polynucleotide members, wherein these members have sequence identity with each other which allows them to recombine in vivo, such as in a host cell, to yield a single self-guiding integration construct comprising:
a guide-RNA expression cassette, and optionally,
an additional polynucleotide element,
   wherein said guide-RNA expression cassette is capable of expressing a functional guide-RNA, or a part thereof, that is specific for a target sequence in a target genome, wherein the part of the self-guiding integration construct comprising said guide-RNA expression cassette and said optional additional polynucleotide element is flanked at its 5′-terminus by a first polynucleotide and at its 3′-terminus by a second polynucleotide, wherein said first and second polynucleotide have sequence identity with sequences flanking the target sequence in the target genome, and wherein the functional guide-RNA, or the part thereof, is encoded by a polynucleotide on the guide-RNA expression cassette and said polynucleotide is operably linked to an RNA polymerase II promoter, to an RNA polymerase III promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter. A non-limiting example of such composition as disclosed herein yielding a self-guiding integration construct according as disclosed herein is depicted in FIGS. 15A-15L. Preferably, a first of the two or more polynucleotide members has a part on its 5′-end that has sequence identity with a part on the 3′-end of a second of the two or more polynucleotide members and so forth, such that a self-guiding integration construct as disclosed herein can be assembled in vivo (within a cell). In a specific embodiment, the polynucleotide members do not have sequence identity with each other but a separate single-stranded or double-stranded oligonucleotide is provided that has sequence identity with both polynucleotide members and allows assembly in vivo (within a cell) of a self-guiding integration construct as disclosed herein.

In the context of all embodiments of the invention, the self-guiding integration construct is a polynucleotide construct, which is not an autonomously replicating entity; it does not comprise an autonomously replicating sequence. The self-guiding integration construct can be a linear or a circular construct and can, in an embodiment, be formed in vivo (within a cell) by recombination of two or more separate, preferably linear members. The term polynucleotide is defined in the “General Definitions” herein.

In all embodiments of the invention, the self-guiding integration construct is preferably a linear self-guiding integration construct. Linear has the meaning as known in the art for a polynucleotide; it is to be construed that the polynucleotide is not circular, has two clearly defined ends, a 5′-end and a 3′-end, which ends are preferably both blunt ends. A linear self-guiding integration construct as disclosed herein may be de novo synthesized, it may be generated by e.g. PCR or by digestion by a restriction enzyme from a vector, such as a plasmid, from a library or other system. A guide-RNA expression cassette as disclosed herein is a polynucleotide expression construct that comprises the components, except for the RNA polymerase, needed to express a functional guide-RNA or a part thereof in vivo such as within a cell. The components include, but are not limited to, a promoter, a coding sequence encoding a guide-RNA or a part thereof and a terminator. Such components are known to the person skilled in the art and are preferably those as defined herein. The “part thereof” of the guide-RNA is preferably the part that comprises or consists of the guide-sequence. The guide-sequence is the recognition sequence, i.e. the sequence that is specific, i.e. substantially complementary, for the target sequence in the target genome and that allows targeting of a complex of a functional polynucleotide-guided genome editing enzyme and a functional guide-RNA to the target sequence in the target genome. The term “specific” in the context of the guide-sequence in the guide-RNA or part thereof, is to be construed that the guide-sequence is substantially complementary to the target sequence in the target genome, wherein “substantially complementary” means that there is sufficient complementarity (sequence identity) between target sequence and guide-sequence to allow hybridization under physiological conditions in a cell; in general one or two mismatches are allowed to still allow sufficient hybridization. The degree of complementarity (sequence identity), when optimally aligned using a suitable alignment algorithm, is preferably higher than 50%, 60%, 70%, 75%, 80%, 85%, 90%, 95%, 96%, 97%, 98% or higher than 99%. Different sequences can guide nucleases, like guide-RNA's for Cas9 (Mali et al., 2013; Cong et al., 2013), crRNA's for Cpf1 (Zetsche et al., 2015) or 5′ phosphorylated single-stranded guide DNA for NgAgo (Gao et al., 2016) as known to the person skilled in the art. When the coding sequence in the self-guiding integration construct does not encode a complete and functional guide-RNA, but encodes the part of the guide-RNA that comprises or consists of the guide-sequence, the other, parts of the guide-RNA that together with the guide-sequence form a functional guide-RNA are encoded on a different construct or are present as such within the cell. The construct encoding the remaining components of the guide-RNA may be present in the genome or may be present on a vector or may be present as such in the cell.

A functional polynucleotide-guided genome editing enzyme can be any system known to the person skilled in the art. Suitable functional genome editing systems for use in all embodiments of the invention include: RNA-guided endonucleases like CRISPR/Cas (Mali et al., 2013; Cong et al., 2013) or CRISPR/Cpf1 (Zetsche et al., 2015) and DNA-guided endonuclease and/or argonaute systems (Gao et al., 2016). The functional genome editing enzyme is preferably a heterologous enzyme, and preferably is an enzyme such as a Cas enzyme, preferably Cas9 or Cas9 nickase; a Cpf1.

The part of the self-guiding integration construct comprising the guide-RNA expression cassette and (optionally) the additional polynucleotide element is flanked at its 5′-terminus by a first polynucleotide and at its 3′-terminus by a second polynucleotide wherein said first and second polynucleotide have sequence identity with sequences flanking the target sequence in the target genome. A non-limiting example of such construct is depicted in FIGS. 15A-15L. Flanked at its 5′-terminus by a first polynucleotide is to be construed as that the first polynucleotide is located immediately adjacent to the 5′-terminal side of the part comprising the guide-RNA expression cassette and the optional additional polynucleotide element. The first polynucleotide may also be referred to as the 5′-flank. Likewise, flanked at its 3′-terminus by a second polynucleotide is to be construed as that the second polynucleotide is located immediately adjacent at the 3′-terminal side of the part comprising the guide-RNA expression cassette and the optional additional polynucleotide element. The second polynucleotide may also be referred to as the 3′-flank. For the avoidance of doubt, the construct is a single polynucleotide wherein the part: 5′-flank-part comprising the guide-RNA expression cassette and the optional additional polynucleotide element-3′-flank are recognizable but comprised of a single string of consecutive nucleotides. The first polynucleotide (5′-flank) and second polynucleotide (3′-flank) have sequence identity with sequences flanking the target sequence in the target genome. The sequence identity of the 5′-flank and 3′-flank in the self-guiding integration construct as disclosed herein is preferably such that the flanks and the sequences flanking the target sequence in the target genome can recombine in vivo such as within a cell such that the self-guiding integration construct according to the invention integrates into the target genome. The person skilled in the art knows that some mismatches are allowed while still allowing recombination. Preferably, the sequence identity of the 5′-flank and 3′-flank in the self-guiding integration construct as disclosed herein and the corresponding sequences flanking the target sequence in the target genome is at least 80, 81, 82, 83, 84, 85, 86, 87, 88, 89, 90, 91, 92, 93, 94, 95, 97, 98 or 99% and most preferably 100%. The 5′-flank and 3′-flank according to the invention may have any length as long as allowing recombination in vivo such as within a cell such that the self-guiding integration construct as disclosed herein integrates into the target genome. Preferably, a 5′-flank has a length of at least 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900 or 1000 nucleotides. Preferably, a 5′-flank has a length of at most 1000, 900, 800, 700, 600, 500, 450, 400, 350, 300, 250, 200, 190, 180, 170, 160, 150, 140, 130, 120, 110, 100, 95, 90, 85, 80, 75, 70, 65, 60, 55, 50, 45, 40, 35, 30 or 25 nucleotides. Preferably, a 3′-flank has a length of at least 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900 or 1000 nucleotides. Preferably, a 3′-flank has a length of at most 1000, 900, 800, 700, 600, 500, 450, 400, 350, 300, 250, 200, 190, 180, 170, 160, 150, 140, 130, 120, 110, 100, 95, 90, 85, 80, 75, 70, 65, 60, 55, 50, 45, 40, 35, 30 or 25 nucleotides.

Preferably, a 5′-flank has a length of from about 25 to about 80 nucleotides, more preferably from about 30 to about 80 nucleotides, more preferably from about 50 to about 80 nucleotides.

Preferably, a 3′-flank has a length of from about 25 to about 80 nucleotides, more preferably from about 30 to about 80 nucleotides, more preferably from about 50 to about 80 nucleotides.

Preferably, a 5′-flank has a length of from 25 to 80 nucleotides, more preferably from 30 to 80 nucleotides, more preferably from 50 to 80 nucleotides. Preferably, a 3′-flank has a length of from 25 to 80 nucleotides, more preferably from 30 to 80 nucleotides, more preferably from 50 to 80 nucleotides.

Preferably, a 5′-flank has a length of from 25 to 80 nucleotides, such as 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80 nucleotides. Preferably, a 3′-flank has a length of from 15 to 80 nucleotides, such as 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80 nucleotides.

To all aspects and embodiments of the invention, a specific embodiment applies to the part of the self-guiding integration construct comprising the guide-RNA expression cassette and the optional additional polynucleotide element that is flanked at its 5′-terminus by a first polynucleotide and at its 3′-terminus by a second polynucleotide, and wherein said first and second polynucleotide have sequence identity with sequences flanking the target sequence in the target genome (see FIG. 17A).

Included in the invention is a provision where two or more self-guiding integration constructs (SGICs) are provided comprising the same guide-RNA expression cassette and an optional additional polynucleotide element, that is/are flanked at its 5′-terminus by a first polynucleotide and at its 3′-terminus by a second polynucleotide, and wherein said first and second polynucleotide have sequence identity with sequences flanking the target sequence in the target genome which are different for each of the two or more SGICs (see FIG. 17E).

Included in the invention is a provision where two or more self-guiding integration constructs (SGICs) are provided each comprising a different guide-RNA expression cassette and an optional additional polynucleotide element, that is/are flanked at its 5′-terminus by a first polynucleotide and at its 3′-terminus by a second polynucleotide, and wherein said first and second polynucleotide have sequence identity with sequences flanking the target sequence in the target genome which are the same for each of the two or more SGICs (see FIG. 17I). In this embodiment, the frequency of NHEJ repair is reduced since if a break mediated by the first SGIC and a polynucleotide-guided editing enzyme is repaired by NHEJ, a target site for a further SGIC will remain present. In such iteration, the chance of NHEJ will be the square of the chance on NHEJ for a single SGIC mediated editing event.

Included in the invention is a provision where the 5′-flank and/or the 3′-flank and the corresponding sequences in the target genome flanking the target sequence, are located on separate single-stranded or double-stranded oligonucleotides (also referred to as ssODN's and dsODN's, respectively; see EP16181781.2, which is herein incorporated by reference) (see FIGS. 17B, 17C, 17D, 17F, 17G, 17H and 17J). In such case, a single-stranded or double-stranded oligonucleotide has a part (i.e. a portion of polynucleotide sequence) that has sequence identity with the part of the self-guiding integration construct comprising the guide-RNA expression cassette and the optional additional polynucleotide element and has a part that has sequence identity with a sequence in the target genome flanking the target sequence. In a typical example, to which the invention is not limited, a first single-stranded or double stranded oligonucleotide has a part that has sequence identity with a sequence on the 5′-end of the part of the self-guiding integration construct comprising the guide-RNA expression cassette and the optional additional polynucleotide element and has a part that has sequence identity with a sequence in the genome that is located 5′ of the target sequence; and, a second single-stranded or double stranded oligonucleotide has a part that has sequence identity with a sequence on the 3′-end of the part of the self-guiding integration construct comprising the guide-RNA expression cassette and the optional additional polynucleotide element and has a part that has sequence identity with a sequence in the genome that is located 3′ of the target sequence (See FIG. 17). In this specific embodiment applying to all embodiments of the invention, the single-stranded oligonucleotide(s) and/or double-stranded oligonucleotide(s) mediate the in vivo (within a cell) integration of the self-guiding integration construct into the target genome. In this specific embodiment applying to all embodiments of the invention, the teachings of WO2017037304 on in vitro assembly of a polynucleotide construct can conveniently be used.

The target sequence in the target genome in a cell is the place where the complex of a functional polynucleotide-guided genome editing enzyme and a guide-RNA binds to and where, if applicable, a double-stranded break or single-stranded break (nick) is created (induced).

The sequences flanking the target sequence in the target genome that have sequence identity with the 5′-flank and with the 3′-flank of the SGIC may be located immediately adjacent to the place where the double-stranded break or single-stranded break is to be induced. In this case, there is overlap between the sequence of the target sequence and those of the sequences flanking the target sequence in the target genome. As a result of the location sequences flanking the target sequence in the target genome immediately adjacent to the induced double-stranded break or single-stranded break, said self-guiding integration construct will integrate at the site of the double-stranded or single-stranded break. The sequences flanking the target sequence in the target genome that have sequence identity with the 5′-flank and with the 3′-flank may also be located away from the place where the double-stranded or single-stranded break is to be induced. The sequence flanking the target sequence in the genome that has sequence identity with the 5′-flank of the self-guiding integration construct according to the invention may be at about 1, 2, 3, 4, 5, 6, 7, 8, 9, 10, 15, 20, 30, 40, 50 100, 200, 300, 400, 500, 1000, 5000, 10000, 50000, 100000 or 200000 nucleotides away from the place where the double-stranded break or single-stranded break is to be induced. The sequence flanking the target sequence in the genome that has sequence identity with the 3′-flank of the self-guiding integration construct according to the invention may be at about 1, 2, 3, 4, 5, 6, 7, 8, 9, 10, 15, 20, 30, 40, 50 100, 200, 300, 400, 500, 1000, 5000, 10000, 50000, 100000 or 200000 nucleotides away from the place where the double-stranded break or single-stranded break is to be induced.

The guide-RNA expression cassette as disclosed herein is, as set forward here above, a polynucleotide expression construct that comprises all components, except for the RNA polymerase, needed to express a functional guide-RNA or a part thereof in vivo such as within a cell. The components include, but are not limited to, a promoter, a coding sequence encoding a guide-RNA or a part thereof and a terminator. There are several ways to express a guide-RNA in vivo, such as within a cell. The guide-RNA may be expressed from an RNA polymerase II promoter. Such promoter is known to the person skilled in the art. Preferred RNA polymerase II promoters are listed in WO2016/50136, WO2016/50135 and WO2016/110453. The guide-RNA may be expressed from RNA polymerase III promoter. Such a promoter is known to the person skilled in the art. Preferred RNA polymerase III promoters are listed in WO2016/50136, WO2016/50135 and WO2016/110453. When using an RNA polymerase III promoter, a self-processing ribozyme is preferably used to convert the raw transcription product into a mature guide-RNA. The guide-RNA may be expressed from a single-subunit DNA-dependent RNA polymerase promoter. Such promoter is known to the person skilled in the art. Preferred single-subunit DNA-dependent RNA polymerase promoters are viral single-subunit DNA-dependent RNA polymerase promoters, such as a T3, SP6, K11 or T7 RNA polymerase promoter. Such preferred single-subunit DNA-dependent RNA polymerase promoters are listed in US62/399,127.

The additional polynucleotide element may be any suitable additional polynucleotide element, functional or non-functional. Preferably, in the self-guiding integration construct according to the invention, or the composition according to the invention, the additional polynucleotide element is a donor polynucleotide, preferably a control sequence, a marker, a gene of interest encoding a compound of interest as defined elsewhere herein, or a disruption construct. The control sequence may be any control sequence or combination of control sequences, such as a promotor, a KOZAK sequence, a signal sequence, a terminator, a pre-sequence, a pre-pro-sequence, a leader sequence, an activator sequence, a repressor sequence, a HIS-tag, a split-GFP tag or any other N-terminal tag. A preferred control sequence is a promoter sequence. This e.g. enables to insert a promoter or to replace an endogenous promoter, or a part thereof, by another promoter. The introduced promoter may be stronger or weaker than the endogenous promoter and/or may be an inducible promoter. Such promoters are known to the person skilled in the art. The marker may be any type of marker as long as it can be identified and thus serves as a marker. The marker may e.g. be a selection marker or may e.g. be an identifiable polynucleotide with known sequence to be used as a barcode or may be a tag such as a HIS-tag, GFP-tag, split GFP-tag, solubility tag. It should be noted that the self-guiding integration construct itself already provides a barcode marker due to its unique guide-sequence, which represents a barcode at the site of integration of the self-guiding integration construct. The gene of interest may be any gene of interest and is preferably one as defined in the section “General Definitions”. The gene of interest may be a complete expression construct comprising a promoter, a coding sequence and a terminator, or may at least comprise a coding sequence. The self-guiding integration construct itself is a construct that disrupts the genome at the site of integration; such disruption may have no influence on the host or may have huge impact on the host. In some cases, it may be desired to introduce a sequence as such that will have a disrupting effect such as a strong or weak promoter sequence, a strong or weak terminator sequence, a splice donor or a splice acceptor sequence; such construct can be incorporated in the self-guiding integration construct as an additional polynucleotide element. Since it is not the intention to create gene drives or likewise autonomous gene editing tools, the self-guiding integration construct according to the invention does not comprise an expression construct encoding a polynucleotide-guided genome editing enzyme. Such enzyme is either expressed from a separate expression construct or is added as such.

Within the scope of the embodiments of the invention, it may be desired to remove the self-guiding integration construct according to the invention again from the host cell at a certain point in time. Several tools are available and known to the person skilled in art; these are within the scope of the invention. For such purpose the self-guiding integration construct according to the invention may e.g. comprise a marker that allows counter selection or may comprise cre-lox sites or directs repeats to facilitate deletion of the construct.

The invention further provides for a composition comprising a self-guiding integration construct according to the invention, a composition comprising a library of self-guiding integration constructs according to the invention, a composition according to the invention yielding a self-guiding integration construct according to the invention or a composition according to the invention yielding a library of self-guiding integration constructs according to the invention, further comprising a functional polynucleotide-guided genome editing enzyme or an expression construct capable of expressing a functional polynucleotide-guided genome editing enzyme. Such composition according to the invention can e.g. be used as a stock solution of components or can e.g. be used for introducing the components into a host cell.

The invention further provides for a host cell comprising a self-guiding integration construct according to the invention or comprising a composition according to the invention yielding a self-guiding integration construct according to the invention. The host cell may be any host cell. Preferred host cells are a fungus, an algae, a microalgae or a marine eukaryote, more preferably a yeast cell, a filamentous fungal cell and a Labyrinthulomycetes cell; all as defined herein in the section “General Definitions”. Preferably, the host cell is deficient in a Non-Homologous End Joining (NHEJ) component. A host cell is to be construed as at least one host cell and a self-guiding integration construct according to the invention is to be construed as at least one self-guiding integration construct according to the invention. Within the scope of the invention is thus a population of host cells comprising a library of self-guiding integration constructs according to the invention and preferably comprising 2, 3, 4, 5, 6, 7, 8, 9, 10 or more SGIC. The host cell and the population of host cells are herein referred to as a host cell according to the invention. Preferably, the host cell according to the invention additionally comprises a functional polynucleotide-guided genome editing enzyme or an expression construct capable of expressing a functional polynucleotide-guided genome editing enzyme. Said a functional polynucleotide-guided genome editing enzyme is preferably a functional polynucleotide-guided heterologous genome editing enzyme.

Preferably, in the host cell according to the invention, the self-guiding integration construct is integrated into the genome at the site where the first and second polynucleotide have sequence identity with the sequences flanking the target sequence in the target genome. A set forward here above, the sequences flanking the target sequence in the target genome that have sequence identity with the 5′-flank and with the 3′-flank may be located immediately adjacent to the place where the double-stranded break or single-stranded break is to be induced. In this case, there is overlap between the target sequence and the sequences flanking the target sequence in the target genome. As a result of the location immediately adjacent to the induced double-stranded break or single-stranded break, the self-guiding integration construct according to the invention will integrate at the site of the double-stranded or single-stranded break. The sequences flanking the target sequence in the target genome that have sequence identity with the 5′-flank and with the 3′-flank may also be located away from the place where the double-stranded or single-stranded break is to be induced. The sequence flanking the target sequence in the genome that has sequence identity with the 5′-flank of the self-guiding integration construct according to the invention may be at about 1, 2, 3, 4, 5, 6, 7, 8, 9, 10, 15, 20, 30, 40, 50 100, 200, 300, 400, 500, 1000, 5000, 10000, 50000, 100000 or 200000 nucleotides away from the place where the double-stranded break or single-stranded break is to be induced. The sequence flanking the target sequence in the genome that has sequence identity with the 3′-flank of the self-guiding integration construct according to the invention may be at about 1, 2, 3, 4, 5, 6, 7, 8, 9, 10, 15, 20, 30, 40, 50 100, 200, 300, 400, 500, 1000, 5000, 10000, 50000, 100000 or 200000 nucleotides away from the place where the double-stranded break or single-stranded break is to be induced.

In a second aspect, the invention provides for the use of a self-guiding integration construct comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide (5′-flank) and at its 3′-terminus by a second polynucleotide (3′-flank), wherein said first and second polynucleotide have sequence identity with sequences flanking the target sequence in the target genome, for expression of a functional guide-RNA or part thereof that is specific for a target sequence in a target genome, in a host cell, wherein the functional guide-RNA, or part thereof that is specific for a target sequence in a target genome, is exclusively expressed from the self-guiding integration construct.

In addition, in this aspect the invention provides for the use of a composition comprising two or more polynucleotide members, wherein these members have sequence identity with each other which allows them to recombine in vivo, such as in a host cell, to yield a self-guiding integration construct comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide (5′-flank) and at its 3′-terminus by a second polynucleotide (3′-flank), wherein said first and second polynucleotide have sequence identity with sequences flanking the target sequence in the target genome, for the expression of a functional guide-RNA or part thereof that is specific for a target sequence in a target genome in a host cell, wherein the functional guide-RNA, or part thereof that is specific for a target sequence in a target genome, is exclusively expressed from the self-guiding integration construct.

In this aspect, all features are preferably those as defined in the first aspect of the invention. In the use according to the invention, the functional guide-RNA, or part thereof, according to the invention is exclusively expressed from the self-guiding integration construct, meaning that there is no other guide-RNA expression construct present in the host cell (not in the genome and not on a vector). The guide-RNA, or part thereof that is specific for a target sequence in a target genome, is initially expressed from the self-guiding integration construct. The expressed guide-RNA facilitates induction of a break into the target genome at the target sequence and subsequently the self-guiding integration construct integrates into the target genome.

Preferably, in the use according to the invention, the self-guiding integration construct further comprises a, additional polynucleotide element as defined in the first aspect herein, wherein the additional polynucleotide element preferably is a control sequence, a marker, a gene of interest, or a disruption construct, as defined in the first aspect herein. Said additional polynucleotide element is, when present, located between the guide-RNA expression cassette and the 5′-flank and/or between the guide-RNA expression cassette and the 3′-flank.

Preferably, in the use according to the invention, the functional guide-RNA, or the part hereof, is encoded by a polynucleotide on the guide-RNA expression cassette and said polynucleotide is operably linked to an RNA polymerase II promoter, to an RNA polymerase III promoter or to a single-subunit DNA-dependent RNA polymerase promoter, preferably a viral single-subunit DNA-dependent RNA polymerase promoter, more preferably a T3, SP6, K11 or T7 RNA polymerase promoter, and optionally to a self-processing ribozyme; all as defined in the first aspect of the invention.

In a third aspect, the invention provides for a method for the production of a host cell according to the invention, comprising introducing into the host cell a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide (5′-flank) and at its 3′-terminus by a second polynucleotide (3′-flank), wherein said first and second polynucleotide have sequence identity with sequences flanking the target sequence in the target genome, wherein in the host preferably a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct.

In addition, in this aspect the invention provides for a method for the production of a host cell according to the invention, comprising introducing into the host cell two or more polynucleotide members, wherein these members have sequence identity with each other which allows them to recombine in the host cell to yield a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, wherein in the host preferably a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct. In this aspect, all features are preferably those as defined in the first aspect herein. In the method according to the invention, the functional guide-RNA, or part thereof, according to the invention is exclusively expressed from the self-guiding integration construct, meaning that there is no other guide-RNA expression construct present in the host cell (not in the genome and not on a vector). The guide-RNA, or part thereof that is specific for a target sequence in a target genome, is initially expressed from the self-guiding integration construct. The expressed guide-RNA facilitates induction of a break into the target genome at the target sequence and subsequently the self-guiding integration construct integrates into the target genome.

Preferably, in the method according to the invention, the self-guiding integration construct further comprises an additional polynucleotide element a defined in the first aspect herein, wherein the additional polynucleotide element preferably is a control sequence, a marker, a gene of interest, or a disruption construct, as defined in the first aspect herein. Said additional polynucleotide element is, when present, located between the guide-RNA expression cassette and the 5′-flank and/or between the guide-RNA expression cassette and the 3′-flank.

Preferably, in the method according to the invention, the functional guide-RNA, or the part hereof, is encoded by a polynucleotide on the guide-RNA expression cassette and said polynucleotide is operably linked to an RNA polymerase II promoter, to an RNA polymerase III promoter or to a single-subunit DNA-dependent RNA polymerase promoter, preferably a viral single-subunit DNA-dependent RNA polymerase promoter, more preferably a T3, SP6, K11 or T7 RNA polymerase promoter, and optionally to a self-processing ribozyme; all as defined in the first aspect of the invention.

A host cell is to be construed as at least one host cell and a self-guiding integration construct according to the invention is to be construed as at least one self-guiding integration construct according to the invention. Accordingly, in an embodiment of the method according to the invention, a library of a self-guiding integration constructs is introduced into a population of host cells. Such method can conveniently be used for screening purposes.

In an embodiment, the method according to the invention further comprises a step determining whether and/or where the self-guiding integration construct has integrated. Such step may be performed using any technique known to the person skilled in the art, such as but not limited to PCR analysis and sequencing such as next generation sequencing allowing easy screening when using libraries of a self-guiding integration constructs. Preferably, the determination is made by analysis of a gene product produced by the generated host cell, preferably by using selective growth conditions. Such selective growth conditions may e.g. allow for the positive selection of a host with the property of interest, allowing screening of a population of host cells wherein a library of self-guiding integration constructs has been introduced. The gene product may e.g. be a metabolite, enzyme (such as glucoamylase or an enzyme that resolves an auxotrophy) or a marker). Preferably, in this aspect of the invention, the host cell that is generated and has properties of interest, is isolated.

In addition, in this aspect the invention provides for a host cell obtainable or a host cell obtained by a method according to the invention. Preferably, such host cell according to the invention comprises a polynucleotide encoding a compound of interest. Said compound of interest is preferably one as defined in the section “General Definitions”. Preferably, said host cell according to the invention expresses the compound of interest. Also provided is the offspring of a host cell obtainable or obtained by a method according to the invention. Such offspring can be generated by culturing and/or by further manipulation of the host cell according to the invention.

Further provided is a method for the production of a compound of interest, comprising culturing the host cell according to this aspect of the invention under conditions conducive to the production of the compound of interest, and, optionally, purifying or isolating the compound of interest. The compound of interest may be any compound of interest, preferably one as defined in the section “General Definitions”. Purification and isolation of the compound of interest may be performed using any technique known to the person skilled in the art.

### EMBODIMENTS

The following embodiments of the invention are provided; the features in these embodiments are preferably those as defined previously herein.

1. A self-guiding integration construct comprising:
a guide-RNA expression cassette, and
an additional polynucleotide element,
   wherein said guide-RNA expression cassette is capable of expressing a functional guide-RNA, or a part thereof, that is specific for a target sequence in a target genome, wherein the part of the self-guiding integration construct comprising said guide-RNA expression cassette and said additional polynucleotide element is flanked at its 5′-terminus by a first polynucleotide and at its 3′-terminus by a second polynucleotide, and wherein said first and second polynucleotide have sequence identity with sequences flanking the target sequence in the target genome.

2. A self-guiding integration construct comprising:
a guide-RNA expression cassette, and optionally,
a additional polynucleotide element, wherein said guide-RNA expression cassette is capable of expressing a functional guide-RNA, or a part thereof, that is specific for a target sequence in a target genome, wherein the part of the self-guiding integration construct comprising said guide-RNA expression cassette and optionally said additional polynucleotide element is flanked at its 5′-terminus by a first polynucleotide and at its 3′-terminus by a second polynucleotide, wherein said first and second polynucleotide have sequence identity with sequences flanking the target sequence in the target genome, and wherein the functional guide-RNA, or the part thereof, is encoded by a polynucleotide on the guide-RNA expression cassette and said polynucleotide is operably linked to an RNA polymerase II promoter, to an RNA polymerase Ill promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter.

3. A self-guiding integration construct comprising:

two or more polynucleotides capable of recombining with each other to yield a guide-RNA expression cassette, and optionally, an additional polynucleotide element,
wherein said guide-RNA expression cassette is capable of expressing a functional guide-RNA, or a part thereof, wherein said functional guide-RNA or part thereof is specific for a target sequence in a target genome, wherein the part of the self-guiding integration construct comprising said guide-RNA expression cassette and optionally said additional polynucleotide element is flanked at its 5′-terminus by a first polynucleotide and at its 3′-terminus by a second polynucleotide, and wherein said first and second polynucleotide have sequence identity with sequences flanking the target sequence in the target genome.

4. A self-guiding integration construct according to any one of embodiments 1-3, wherein the self-guiding integration construct is a linear self-guiding integration construct.

5. A composition comprising two or more polynucleotide members, wherein these members have sequence identity with each other which allows them to recombine in vivo, such as in a host cell, to yield a single self-guiding integration construct according to embodiment 1 or 2 or to yield a linear self-guiding integration construct according to embodiment 4.

6. The self-guiding integration construct according to embodiment 1-4, or the composition according to embodiment 5, wherein the additional polynucleotide element is a control sequence, a marker, a gene of interest, or a disruption construct.

7. A composition comprising a self-guiding integration construct as defined in any one of embodiments 1-4, or the composition according to embodiment 5, preferably comprising a library of self-guiding integration constructs, said composition preferably further comprising a functional polynucleotide-guided genome editing enzyme or an expression construct capable of expressing a functional polynucleotide-guided genome editing enzyme.

8. A host cell comprising a self-guiding integration construct as defined in any one of embodiments 1-4 or 6, or the composition according to embodiment 5.

9. A host cell according to embodiment 8, further comprising a functional polynucleotide-guided genome editing enzyme, preferably a functional polynucleotide-guided heterologous genome editing enzyme, or further comprising an expression construct capable of expressing a functional polynucleotide-guided genome editing enzyme, preferably a functional polynucleotide-guided heterologous genome editing enzyme.

10. A host cell according to embodiment 8 or 9, wherein the self-guiding integration construct is integrated into the genome at the site where the first and second polynucleotide have sequence identity with the sequences flanking the target sequence in the target genome.

11. Use of a self-guiding integration construct comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, for expression of a functional guide-RNA or part thereof that is specific for a target sequence in a target genome, in a host cell, wherein the functional guide-RNA, or part thereof that is specific for a target sequence in a target genome, is exclusively expressed from the self-guiding integration construct.

12. Use of a composition comprising two or more polynucleotide members, wherein these members have sequence identity with each other which allows them to recombine in vivo, such as in a host cell, to yield a self-guiding integration construct comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, for the expression of a functional guide-RNA or part thereof that is specific for a target sequence in a target genome in a host cell, wherein the functional guide-RNA, or part thereof that is specific for a target sequence in a target genome, is exclusively expressed from the self-guiding integration construct.

13. Use according to embodiment 11 or 12, wherein the self-guiding integration construct is a linear self-guiding integration construct.

14. Use according to any one of embodiments 11-13, wherein the self-guiding integration construct further comprises an additional polynucleotide element, wherein the donor polynucleotide preferably is a control sequence, a marker, a gene of interest, or a disruption construct.

15. Use according to any one of embodiments 11-14, wherein the functional guide-RNA, or the part hereof, is encoded by a polynucleotide on the guide-RNA expression cassette and said polynucleotide is operably linked to an RNA polymerase II promoter, to an RNA polymerase Ill promoter or to a single-subunit DNA-dependent RNA polymerase promoter, preferably a viral single-subunit DNA-dependent RNA polymerase promoter, more preferably a T3, SP6, K11 or T7 RNA polymerase promoter, and optionally to a self-processing ribozyme.

16. A method for the production of a host cell, comprising introducing into the host cell a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, wherein in the host preferably a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct.

17. A method for the production of a host cell, comprising introducing into the host cell two or more polynucleotide members, wherein these members have sequence identity with each other which allows them to recombine in the host cell to yield a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, wherein in the host preferably a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct.

18. The method according to embodiment 16 or 17, wherein the self-guiding integration construct is a linear self-guiding integration construct.

19. The method according to any one of embodiments 16-18, wherein the self-guiding integration construct further comprises an additional polynucleotide element, wherein the additional polynucleotide element preferably is a control sequence, a marker, a gene of interest, or a disruption construct.

20. The method according to any one of embodiments 16-19, wherein the functional guide-RNA, or the part thereof, is encoded by a polynucleotide on the guide-RNA expression cassette and said polynucleotide is operably linked to an RNA polymerase II promoter, to an RNA polymerase Ill promoter or to a single-subunit DNA-dependent RNA polymerase promoter, preferably a viral single-subunit DNA-dependent RNA polymerase promoter, more preferably a T3, SP6, K11 or T7 RNA polymerase promoter, and optionally to a self-processing ribozyme.

21. The method according to any one of embodiments 16-20, wherein a library of a self-guiding integration constructs is introduced into a population of host cells.

22. The method according to any one of embodiments 16-21, further comprising determining whether and/or where the self-guiding integration construct has integrated.

23. The method according to embodiment 22, wherein the determination is made by analysis of a gene product produced by the generated host cell, preferably by using selective growth conditions.

24. A host cell according to any one of embodiments 8-10 or a cell obtainable or obtained by a method according to any one of embodiments 16-23, said cell comprising a polynucleotide encoding a compound of interest.

25. The host cell according to embodiment 24, expressing the compound of interest.

26. A method for the production of a compound of interest, comprising culturing the cell according to embodiment 24 or 25 under conditions conducive to the production of the compound of interest, and, optionally, purifying or isolating the compound of interest.

### General Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value. CRISPR interference (CRISPRi) is a genetic perturbation technique that allows for sequence-specific repression or activation of gene expression in prokaryotic and eukaryotic cells.

Herein, the term “in vivo” is used as meaning within an individual cell, said individual cell not being part of a multicellular higher eukaryotic organism such as an animal, including a human. Herein, the term “ex vivo” is used as meaning outside the human or animal body.

When herein is mentioned the term “0 kbp” deletion, this is not have to be exactly a 0 kbp deletion; depending on the specifics of the SGIC several base pairs, such as e.g. about 0, 10, 20, 30, 40, 50, 60, 70, 80, 90, 100, 110, 120, 130, 140, 150 or 200 base pairs, will be deleted from the genome upon integration of the SGIC.

A polynucleotide refers herein to a polymeric form of nucleotides of any length or a defined specific length-range or length, of either deoxyribonucleotides or ribonucleotides, or mixes or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, oligonucleotides and primers. A polynucleotide may comprise natural and non-natural nucleotides and may comprise one or more modified nucleotides, such as a methylated nucleotide and a nucleotide analogue or nucleotide equivalent wherein a nucleotide analogue or equivalent is defined as a residue having a modified base, and/or a modified backbone, and/or a non-natural internucleoside linkage, or a combination of these modifications. As desired, modifications to the nucleotide structure may be introduced before or after assembly of the polynucleotide. A polynucleotide may be further modified after polymerization, such as by conjugation with a labeling compound.

In general, codon optimization refers to a process of modifying a nucleic acid sequence for enhanced expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See e.g. Nakamura, Y., et al., 2000. Computer algorithms for codon optimizing a particular sequence for expression in a particular host cell are also available, such as Gene Forge (Aptagen; Jacobus, Pa.), are also available. Preferably, one or more codons (e.g. 1, 2, 3, 4, 5, 10, 15, 20, 25, 50, or more, or all codons) in a sequence encoding a Cas protein correspond to the most frequently used codon for a particular amino acid. Preferred methods for codon optimization are described in WO2006/077258 and WO2008/000632). WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence. The amount of Cas protein in a source in a composition according to the invention may vary and may be optimized for optimal performance. In an RNA molecule with a 5′-cap, a 7-methylguanylate residue is located on the 5′ terminus of the RNA (such as typically in mRNA in eukaryotes). RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase. The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export.

A ribozyme refers to one or more RNA sequences that form secondary, tertiary, and/or quaternary structure(s) that can cleave RNA at a specific site. A ribozyme includes a “self-cleaving ribozyme, or self-processing ribozyme” that is capable of cleaving RNA at a c/s-site relative to the ribozyme sequence (i.e., auto-catalytic, or self-cleaving). The general nature of ribozyme nucleolytic activity is known to the person skilled in the art. The use of self-processing ribozymes in the production of guide-RNA's for RNA-guided nuclease systems such as CRISPR/Cas is inter alia described by Gao et al, 2014.

A nucleotide analogue or equivalent typically comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991. Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005. Chem. Commun, 495-497). Since the backbone of a PNA molecule contains no charged phosphate groups, PNA-RNA hybrids are usually more stable than RNA-RNA or RNA-DNA hybrids, respectively (Egholm et al., 1993. Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage. A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Within the invention, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide according to the invention is represented by a nucleotide sequence. A polypeptide according to the invention is represented by an amino acid sequence. A nucleic acid construct according to the invention is defined as a polynucleotide which is isolated from a naturally occurring gene or which has been modified to contain segments of polynucleotides which are combined or juxtaposed in a manner which would not otherwise exist in nature.

The sequence information as provided herein should not be so narrowly construed as to require inclusion of erroneously identified bases. The skilled person is capable of identifying such erroneously identified bases and knows how to correct for such errors.

A compound of interest in the context of all embodiments of the invention may be any biological compound. The biological compound may be biomass or a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides, the polynucleotide may be a gene, the series of polynucleotide may be a gene cluster. In all embodiments of the invention, the single polynucleotide or series of polynucleotides encoding the biological compound of interest or the biosynthetic or metabolic pathway associated with the biological compound of interest, are preferred targets for the compositions and methods according to the invention. The biological compound may be native to the host cell or heterologous to the host cell.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. The term polypeptide refers to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. Polypeptides further include naturally occurring allelic and engineered variations of the above-mentioned polypeptides and hybrid polypeptides. The polypeptide may be native or may be heterologous to the host cell. The polypeptide may be a collagen or gelatine, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

According to the invention, a compound of interest can be a polypeptide or enzyme with improved secretion features as described in WO2010/102982. According to the invention, a compound of interest can be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933. The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). In a preferred option, the polysaccharide is hyaluronic acid. A polynucleotide coding for the compound of interest or coding for a compound involved in the production of the compound of interest according to the invention may encode an enzyme involved in the synthesis of a primary or secondary metabolite, such as organic acids, carotenoids, (beta-lactam) antibiotics, and vitamins. Such metabolite may be considered as a biological compound according to the invention.

The term “metabolite” encompasses both primary and secondary metabolites; the metabolite may be any metabolite. Preferred metabolites are citric acid, gluconic acid, adipic acid, fumaric acid, itaconic acid and succinic acid.

A metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

A primary metabolite may be, but is not limited to, an amino acid, fatty acid, nucleoside, nucleotide, sugar, triglyceride, or vitamin.

A secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

According to the invention, a compound of interest is preferably a polypeptide as described in the list of compounds of interest.

According to another embodiment of the invention, a compound of interest is preferably a metabolite.

A cell according to the invention may already be capable of producing a compound of interest. A cell according to the invention may also be provided with a homologous or heterologous nucleic acid construct that encodes a polypeptide wherein the polypeptide may be the compound of interest or a polypeptide involved in the production of the compound of interest. The person skilled in the art knows how to modify a microbial host cell such that it is capable of producing a compound of interest.

All embodiments of the invention refer to a cell, not to a cell-free in vitro system; in other words, the systems according to the invention are cell systems, not cell-free in vitro systems.

In all embodiments of the invention, e.g., the cell according to the invention may be a haploid, diploid or polyploid cell.

A cell according to the invention is interchangeably herein referred as “a cell”, “a cell according to the invention”, “a host cell”, and as “a host cell according to the invention”; said cell may be any cell, a prokaryotic or a eukaryotic cell. Preferably, the cell is not a mammalian cell. Preferably the cell is a fungus, i.e. a yeast cell or a filamentous fungus cell. Preferably, the cell is deficient in an NHEJ (non-homologous end joining) component. Said component associated with NHEJ is preferably a homologue or orthologue of the yeast Ku70, Ku80, MRE11, RAD50, RAD51, RAD52, XRS2, SIR4, and/or LIG4. Alternatively, in the cell according to the invention NHEJ may be rendered deficient by use of a compound that inhibits RNA ligase IV, such as SCR7 (Vartak S V and Raghavan, 2015). The person skilled in the art knows how to modulate NHEJ and its effect on RNA-guided nuclease systems, see e.g. WO2014130955A1; Chu et al., 2015; et al., 2015; Song et al., 2015 and Yu et al., 2015; all are herein incorporated by reference. The term “deficiency” is defined elsewhere herein.

When the cell according to the invention is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Pichia stipidis and Pichia pastoris.

The host cell according to the invention is a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51.2011). In another preferred embodiment, the filamentous fungal host cell may be a cell of any filamentous form of any of the three families Aspergillaceae, Thermoascaceae and Trichocomaceae, which are accommodated in the taxon Trichocomaceae.

The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell according to the invention is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255 (ATCC28089); even more preferably the filamentous fungal host cell according to the invention is an Aspergillus niger. When the host cell according to the invention is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells according to the present invention are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255 (ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila C1, Garg 27K, VKM-F 3500 D, Chrysosporium lucknowense C1, Garg 27K, VKM-F 3500 D, ATCC44006 and derivatives thereof.

Preferably, a host cell according to the invention has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In an embodiment the host cell according to the invention comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32 (7) or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the The Altered Sites® II in vitro Mutagenesis Systems' (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified. Alternatively or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K, Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52).

A modification resulting in reduced or no production of undesired compound is preferably due to a reduced production of the mRNA encoding said undesired compound if compared with a parent microbial host cell which has not been modified and when measured under the same conditions. A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in e.g. WO2008/053019, WO2005/05672A1 and WO2005/026356A1.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath_et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment of the invention, the modification in the genome of the host cell according to the invention is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell according to the invention produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The invention is further illustrated by the following examples:

### EXAMPLES

In the following Examples, various embodiments of the invention are illustrated. From the above description and these Examples, one skilled in the art can make various changes and modifications of the invention to adapt it to various usages and conditions.

### Example 1: SGIC in S. cerevisiae

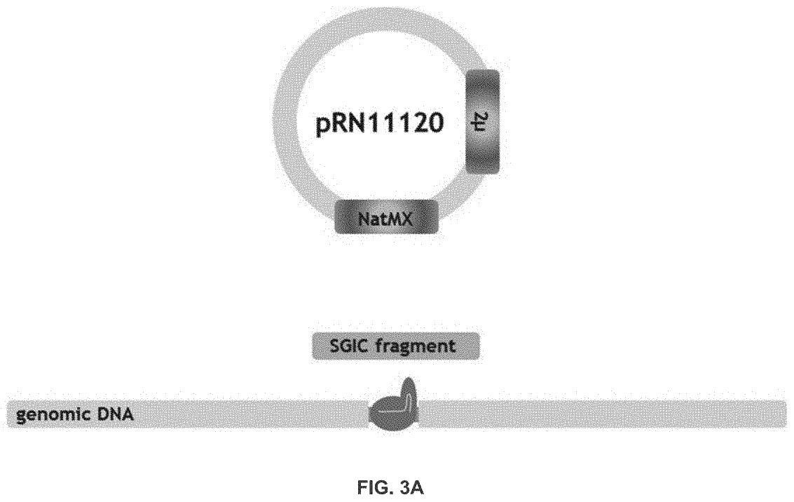

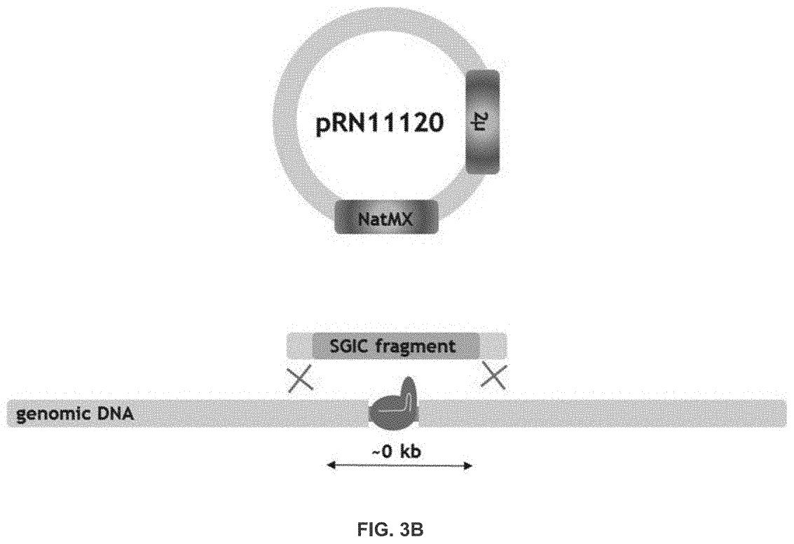

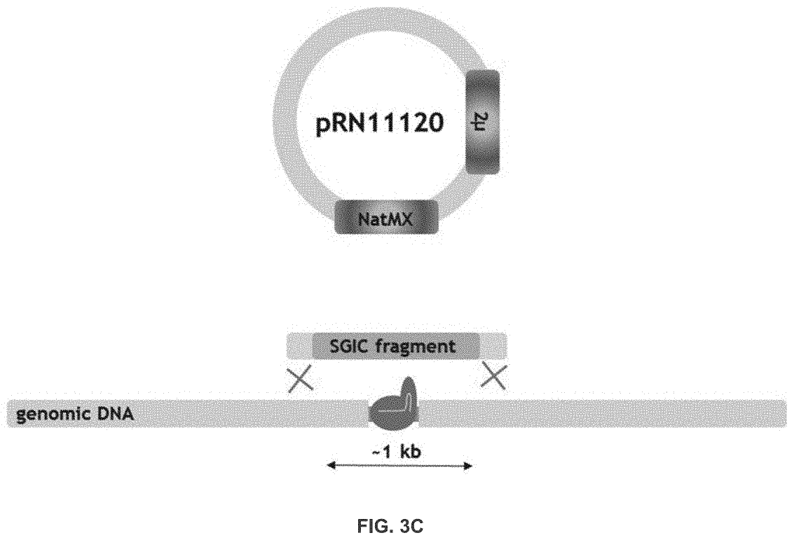

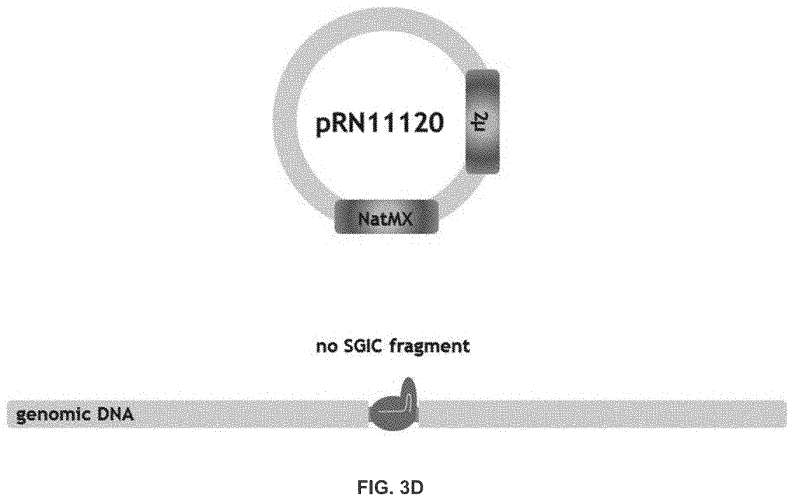

This example describes the integration of a Self-Guiding Integration Construct (SGIC) type guide-RNA expression cassette using a CRISPR/Cas9 system in Saccharomyces cerevisiae. The SGIC's comprise 50 bp flanks at both the 5′ and 3′ end with sequence identity with genomic DNA sequences to allow integration via homologous recombination at the desired genomic locus (either INT1, INT59 or YPRCtau3). Depending on the sequence of the flanks, a stretch of DNA of up to 1 kbp is deleted from the genome upon integration of the SGIC. This set-up is visually shown in FIGS. 3A-3D.

In the SGIC's, for the expression of guide-RNA's in S. cerevisiae, a guide-RNA expression cassette with control elements as previously described by DiCarlo et al., 2013 was used. The guide-RNA expression cassettes used in this example comprise the SNR52 promoter, a guide-RNA sequence consisting of the guide-sequence (also referred to as genomic target sequence) and the guide-RNA structural component followed by the SUP4 terminator.

Construction of a Cas9-Expressing Saccharomyces cerevisiae Strain

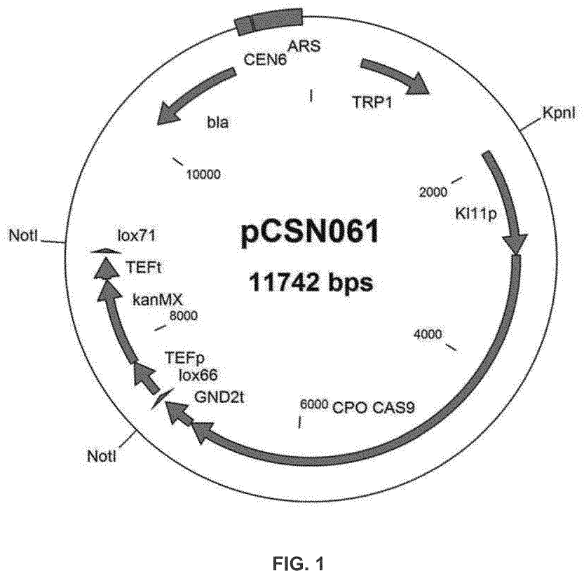

Yeast vector pCSN061 is a single copy vector (CEN/ARS) that contains a Cas9 expression cassette consisting of a Cas9 codon optimized variant (WO2016/110512) expressed from the KI11 promoter (Kluyveromyces lactis promoter of KLLA0F20031g), the S. cerevisiae GND2 terminator, and a functional KanMX marker cassette conferring resistance against G418. The Cas9 expression cassette was KpnI/NotI ligated into pRS414 (Sikorski and Hieter, 1989), resulting in intermediate vector pCSN004. Subsequently, a functional expression cassette conferring G418 resistance (see: www.euroscarf.de) was NotI restricted from vector pUG7-KanMX and NotI ligated into pCSN004, resulting in vector pCSN061 that is depicted in FIG. 1; the sequence is set out in SEQ ID NO: 2.

Vector pCSN061 containing the Cas9 expression cassette was first transformed to S. cerevisiae strain CEN.PK113-7D (MATa URA3 HIS3 LEU2 TRP1 MAL2-8 SUC2) using the LiAc/salmon sperm (SS) carrier DNA/PEG method (Gietz and Woods, 2002). Strain CEN.PK113-7D is available from the EUROSCARF collection (http://www.euroscarf.de, Frankfurt, Germany). The origin of the CEN.PK family of strains is described by van Dijken et al., 2000. In the transformation mixture one microgram of vector pCNS061 was used. The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) per ml. After two to four days of growth at 30° C. transformants appeared on the transformation plate. A transformant conferring resistance to G418 on the plate, further referred to as strain CSN001, was inoculated on YPD-G418 medium (10 grams per liter of yeast extract, 20 grams per liter of peptone, 20 grams per liter of dextrose, 200 μg G418 (Sigma Aldrich, Zwijndrecht, the Netherlands) per ml, was used in subsequent transformation experiments.

### Self-Guiding Integration Construct (SGIC) Type Guide-RNA Expression Cassettes

Synthetic DNA's containing guide-RNA expression cassettes were ordered as synthetic DNA (gBlocks) at Integrated DNA Technologies (IDT, Leuven, Belgium). An overview of the sequences is provided in Table 1. The gBlock DNA's were used as template in a PCR reaction, using primers as indicated in Table 1, and using PrimeSTAR GXL DNA Polymerase (Takara/Cat no. R050A) according to the manufacturer's instructions. The resulting SGIC DNA's, of which the sequences are set out in SEQ ID NO's: 22, 23, 24, 25, 26, 27, 30, 31 and 32, consisted of the SNR52p RNA polymerase III promoter, a guide-sequence (also referred to as genomic target sequence; SEQ ID NO's: 7, 8, 9), the gRNA structural component and the SUP4 3′ flanking region as described in DiCarlo et al., 2013, and include a 50 bp genomic DNA sequence at both the 5′ and 3′ end for integration at the genomic locus being either INT1, INT59 or YPRC tau3. The SGIC DNA's either target approximately directly at the introduced double stranded (ds) break (0 kbp deletion) or at approximately 500 bp upstream and approximately 500 bp downstream of the ds break (1 kbp deletion) DNA. It should be noted that a “0 kbp” deletion is not exactly a “0 kbp”; depending on the specifics of the SGIC several base pairs will be deleted upon integration of the SGIC. Typically, in this example in case of INT1 and YPRCtau3, 130 bp was deleted and in case of INT59, 90 bp was deleted, as determined by sequencing (data not shown).

Control SGIC DNA was also included in the transformation. The control SGIC DNA's contained a functional guide-RNA expression cassette having no homology with genomic S. cerevisiae DNA, i.e. they will not integrate by homologous recombination. The control SGIC DNA sequences are provided in SEQ ID NO: 30 (INT1), SEQ ID NO: 31 (INT59) and SEQ ID NO: 32 (YPRCtau3). DNA templates and primers used to obtain the control SGIC DNA sequences by PCR are listed in Table 1. PCR reactions were performed using PrimeSTAR GXL DNA Polymerase (Takara/Catno. R050A) according to the manufacturer's instructions.

The generated SGIC's were purified using a NucleoSpin Gel and PCR Clean-up kit (Machery-Nagel, distributed by Bioké, Leiden, the Netherlands) according to manufacturer's instructions. Subsequently, DNA concentrations of purified SGIC DNA's were measured using a NanoDrop (ND-1000 Spectrophotometer, Thermo Scientific, Bleiswijk, the Netherlands).

TABLE 1
Overview of the sequences of the SGIC DNA's used in transformation.
The template guide-RNA expression cassettes were used as a template for
PCR using the primers indicated in this table in order to obtain SGIC
DNA's (SGIC DNA fragments) used in the transformation experiments.

| | Template guide- | Guide sequence | | Sequence of |
|---|---|---|---|---|
| | RNA expression | (genomic target | Primers used to | the SGIC DNA |
| Target | cassette | sequence) | obtain SGIC DNA | fragment |
| INT1 site, 0 | SEQ ID NO: 4 | SEQ ID NO: 7 | SEQ ID NO: 10 | SEQ ID NO: 22 |
| kB deletion | | | SEQ ID NO: 11 | |
| INT1 site, 1 | SEQ ID NO: 4 | SEQ ID NO: 7 | SEQ ID NO: 12 | SEQ ID NO: 23 |
| kB deletion | | | SEQ ID NO: 13 | |
| INT59 site, 0 | SEQ ID NO: 5 | SEQ ID NO: 8 | SEQ ID NO: 14 | SEQ ID NO: 24 |
| kB deletion | | | SEQ ID NO: 15 | |
| INT59 site, 1 | SEQ ID NO: 5 | SEQ ID NO: 8 | SEQ ID NO: 16 | SEQ ID NO: 25 |
| kB deletion | | | SEQ ID NO: 17 | |
| YPRCtau3 | SEQ ID NO: 6 | SEQ ID NO: 9 | SEQ ID NO: 18 | SEQ ID NO: 26 |
| site, 0 kB | | | SEQ ID NO: 19 | |
| deletion | | | | |
| YPRCtau3 | SEQ ID NO: 6 | SEQ ID NO: 9 | SEQ ID NO: 20 | SEQ ID NO: 27 |
| site, 1 kB | | | SEQ ID NO: 21 | |
| deletion | | | | |
| INT1 no | SEQ ID NO: 4 | SEQ ID NO: 7 | SEQ ID NO: 28 | SEQ ID NO: 30 |
| flanks control | | | SEQ ID NO: 29 | |
| INT59 no | SEQ ID NO: 5 | SEQ ID NO: 8 | SEQ ID NO: 28 | SEQ ID NO: 31 |
| flanks control | | | SEQ ID NO: 29 | |
| YPRCtau3 | SEQ ID NO: 6 | SEQ ID NO: 9 | SEQ ID NO: 28 | SEQ ID NO: 32 |
| no flanks | | | SEQ ID NO: 29 | |
| control | | | | |

pRN1120 Vector Construction (Multi-Copy Expression Vector, NatMX Marker)

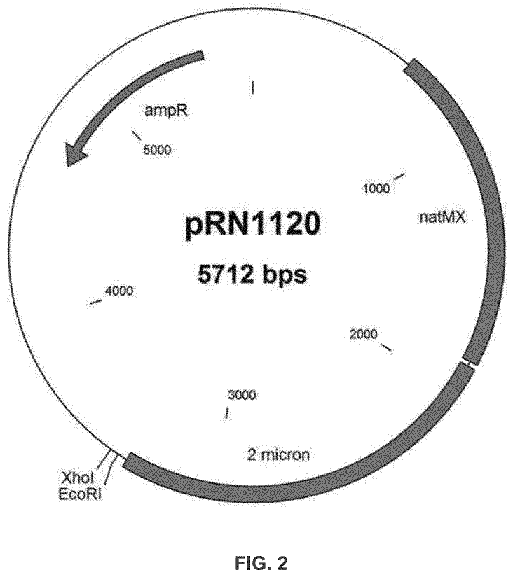

Yeast vector pRN1120 is a multi-copy vector (2 micron) that contains a functional NatMX marker cassette conferring resistance against nourseothricin. The backbone of this vector is based on pRS305 (Sikorski and Hieter, 1989), and includes a functional 2 micron ORI sequence and a functional NatMX marker cassette (see www.euroscarf.de). Vector pRN1120 is depicted in FIG. 2 and the sequence is set out in SEQ ID NO: 3.

### DNA Concentrations

All DNA concentrations, including the guide-RNA expression cassette PCR product and pRN1120, were determined using a NanoDrop device (ThermoFisher, Life Technologies, Bleiswijk, the Netherlands), providing the concentrations in nanogram per microliter. Based on these measurements, an amount of 1 μg SGIC DNA and 10 ng of circular plasmid pRN1120 were used in the transformation experiments.

### Integration Sites

The INT1 integration site is located in the non-coding region between NTR1 (YOR071c) and GYP1 (YOR070c), located on chromosome XV. The INT59 integration site is a non-coding region between SRP40 (YKR092C) and PTR2 (YKR093W) located on chromosome XI. The YPRCtau3 integration site is a Ty4 long terminal repeat, located on chromosome XVI, and has previously been described by Flagfeldt et al. (2009).

### Yeast Transformation

Strain CSN001 which is pre-expressing Cas9, was inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN001 was transformed with 1 μg of SGIC DNA as indicated in Table 2, using the LiAc/SS carrier DNA/PEG method (Gietz and Woods, 2002) and 10 ng vector pRN1120. In transformations #4, #8 and #12 no SGIC DNA was added to the transformation mixture. The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 2
Overview of SGIC DNA's used in the
different transformation experiments.

| | | SGIC DNA | |
|---|---|---|---|
| Transformation | Description | sequence | FIG. |
| #1 | INT1 no flank | SEQ ID NO: 30 | 3A |
| | control | | |
| #2 | INT1 site, 0 kB | SEQ ID NO: 22 | 3B |
| | deletion | | |
| #3 | INT1 site, 1 kB | SEQ ID NO: 23 | 3C |
| | deletion | | |
| #4 | No INT1 SGIC | | 3D |
| #5 | INT59 no flanks | SEQ ID NO: 31 | 3A |
| | control | | |
| #6 | INT59 site, 0 kB | SEQ ID NO: 24 | 3B |
| | deletion | | |
| #7 | INT59 site, 1 kB | SEQ ID NO: 25 | 3C |
| | deletion | | |
| #8 | No INT59 SGIC | | 3D |
| #9 | YPRCtau3 no | SEQ ID NO: 32 | 3A |
| | flanks control | | |
| #10 | YPRCtau3 site, | SEQ ID NO: 26 | 3B |
| | 0 kB deletion | | |
| #11 | YPRCtau3 site, | SEQ ID NO: 27 | 3C |
| | 1 kB deletion | | |
| #12 | No YPRCtau3 | | 3D |
| | SGIC | | |

### Results

The transformation experiment outlined above in Table 2 was performed and after transformation, the cells were plated on YPD selective plates. To confirm correct integration of the SGIC comprising the guide-RNA expression cassette and to demonstrate deletion of 0 kbp and 1 kbp of genomic DNA at the INT1, INT59 or YPRCtau3 locus, 24 transformants of each transformation were analyzed by PCR. Genomic DNA of the transformants was isolated as described by Löoke et al., 2011 and was used as template in a PCR reaction. The primers used to confirm the integration were designed to hybridize in the genome just outside the genomic flanking regions that are present in the SGIC DNA. PCR reactions were performed using MyTag™ Red Mix (Catno BIO-25044, Bioline—Germany) according to manufacturer's instructions and a standard PCR program known to the person skilled in the art. When using the primer sets that are set out in Table 3 in the PCR reaction, correct integration was demonstrated by a PCR product of the size as mentioned in the most right column of Table 3. Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCT products.

TABLE 3
Overview of analysis of transformants by PCR

| | | | Product size: | | Product size: |
|---|---|---|---|---|---|
| | | | no SGIC | | SGIC |
| Transformation | Target | Primer set | Integration | | integration |
| #1, 2, 4 | INT1 0 kb | SEQ ID NO: 33 and | 498 | bp | 756 bp |
| | deletion | SEQ ID NO: 34 | | | |
| # 1, 3, 4 | INT1 1 kb | SEQ ID NO: 35 and | 1342 | bp | 663 bp |
| | deletion | SEQ ID NO: 36 | | | |
| # 5, 6, 8 | INT59 0 kb | SEQ ID NO: 37 and | 280 | bp | 578 bp |
| | deletion | SEQ ID NO: 38 | | | |
| # 5, 7, 8 | INT59 1 kb | SEQ ID NO: 39 and | 1280 | bp | 608 bp |
| | deletion | SEQ ID NO: 40 | | | |
| # 9, 10, 12 | YPRC tau3 0 kb | SEQ ID NO: 41 and | 282 | bp | 540 bp |
| | deletion | SEQ ID NO: 42 | | | |
| # 9, 11, 12 | YPRC tau3 1 kb | SEQ ID NO: 43 and | 1272 | bp | 610 bp |
| | deletion | SEQ ID NO: 44 | | | |

An overview of the results of the PCR reactions performed to analyze transformants for correct integration of SGIC DNA is displayed here below in Table 4. Without genomic DNA flanks at the 5′ and 3′ end of the SGIC, no integration was observed (transformations #1, #5, #9). In this experiment, the success rate for integration of SGIC DNA with combined deletion of 1 kb of the genomic DNA around the integration site was slightly higher (63% at best) compared to the 0 kb deletion (50% at best) wherein the SGIC was integrated at the Cas9 induced double-strand break with deletion of genomic DNA. Overall, the PCR results confirmed integration of the SGIC DNA with a success rate of up to 63%.

TABLE 4
Overview of the results of the colony PCR performed to confirm
integration of the SGIC comprising the guide-RNA expression
cassette at the correct location in the genome.

| | | Number of | Number of | |
|---|---|---|---|---|
| | | transformants | transformants | |
| | Number of | with | without | Percentage |
| Transfor- | transformants | integrated | integrated | edited |
| mation | tested | SGIC | SGIC | cells |
| # 1 | 5 | 0 | 5 | 0% |
| # 2 | 24 | 8 | 16 | 33% |
| # 3 | 24 | 15 | 9 | 63% |
| # 4 | 24 | 0 | 24 | 0% |
| # 5 | 14 | 0 | 14 | 0% |
| # 6 | 24 | 12 | 12 | 50% |
| # 7 | 24 | 14 | 10 | 58% |
| # 8 | 24 | 0 | 24 | 0% |
| # 9 | 24 | 0 | 24 | 0% |
| # 10 | 24 | 7 | 17 | 29% |
| # 11 | 24 | 9 | 15 | 38% |
| # 12 | 24 | 0 | 24 | 0% |

### Example 2: Split SGIC in S. cerevisiae

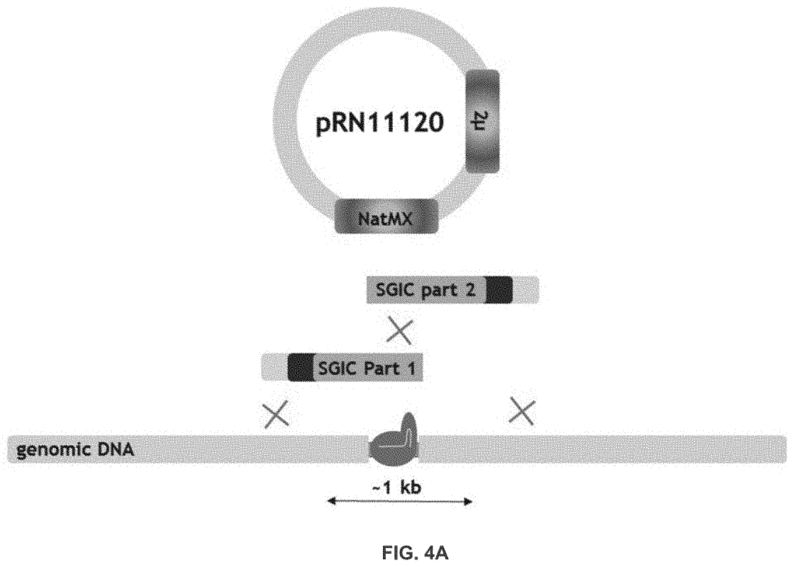

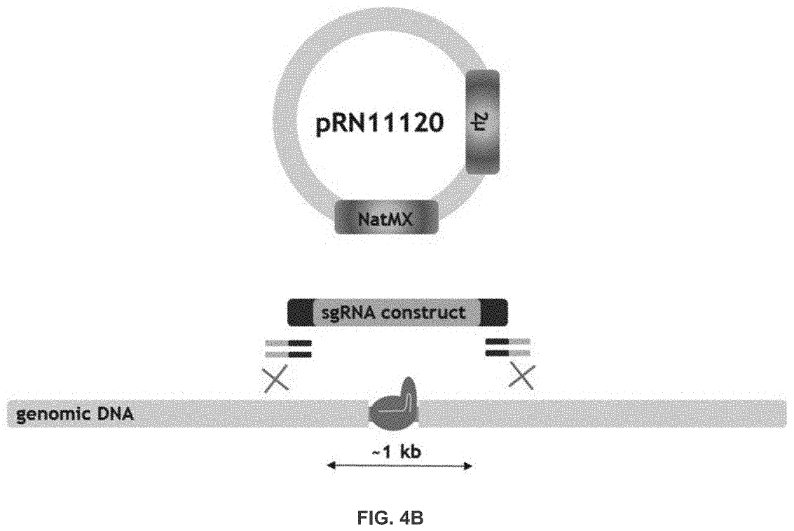

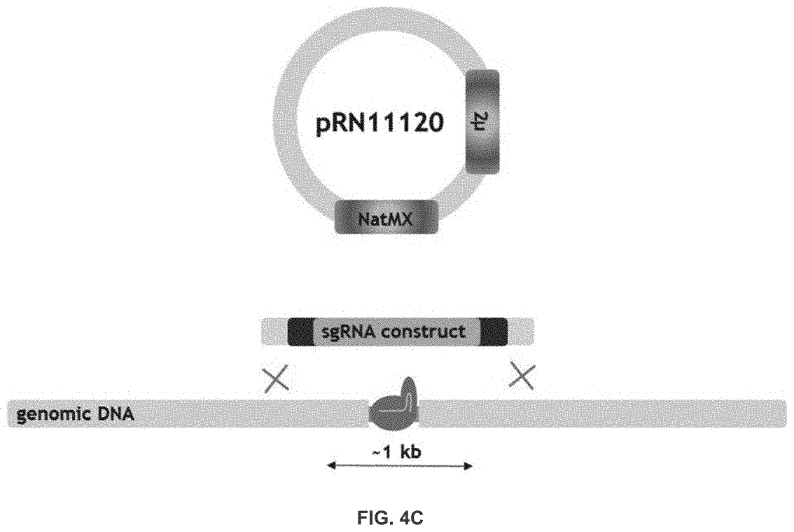

This example describes two SGIC split guide-RNA fragments which are essentially two halves of an SGIC as set forward in Example 1 having a 80 bp overlap homology with each other to allow in vivo (within a yeast cell) assembly in of the functional SGIC. The assembled functional SGIC comprised a guide-RNA expression cassette and 50 bp flanks at both the 5′ and 3′ end with sequence identity with genomic DNA sequences to allow integration via homologous recombination at the desired genomic locus. The functional SGIC comprising the guide-RNA expression cassette was subsequently integrated into the INT1 locus of the S. cerevisiae genome. The experimental set-up is depicted in FIGS. 4A-4C.

### Experimental Details

The components required in this example are as follows:
Yeast strain CSN001 which is pre-expressing Cas9. Construction of the strain CSN001 is described in Example 1.
pRN1120, multi-copy expression vector containing NatMX marker. Construction and details of the plasmid are described in Example 1.
   100 by ssODN Flank Sequences

In a sub-experiment, to target the integration of an SGIC type guide-RNA expression cassette (SEQ ID NO: 47) that has itself no sequence identity with the genomic integration site, single-stranded oligonucleotides of 100 bp each were included in transformation 4B (Table 6). These left flank (LF) and right flank (RF) sequences have 50 bp homology with the 5′-terminus and 3′-terminus of the SGIC and 50 bp homology with the genome. By integration of the SGIC, a stretch of 1 kbp genomic DNA was deleted from the INT1 locus.

The INT1 integration site is located in the non-coding region between NTR1 (YOR071c) and GYP1 (YOR070c), located on chromosome XV.

### Split SGIC's

The guide-RNA expression cassette directing Cas9 to the INT1 integration site was ordered as synthetic DNA (gBlock) at Integrated DNA Technologies (IDT, Leuven, Belgium), SEQ ID NO: 4. This gBlock was used as template in a PCR reaction using primers SEQ ID NO: 45 and SEQ ID NO: 46, resulting in an SGIC flanked by connector sequences on the 5′ and 3′ ends. These connector sequences are random DNA sequences of 50 bp, 5′ connector sequence (SEQ ID NO: 59) and 3′ connector sequence (SEQ ID NO: 60). The resulting PCR product, SEQ ID NO: 47, was used as template in subsequent PCR reactions to obtain split SGIC DNA fragments (SGIC part 1 and SGIC part 2, see FIG. 4A). Primer sets SEQ ID NO: 48 and SEQ ID NO: 50, SEQ ID NO: 49 and SEQ ID NO: 51 were used to obtain the 5′ part and 3′ part of the SGIC, SEQ ID NO: 53 and SEQ ID NO: 54 respectively. PCR product, SEQ ID NO: 47 was also used as template in a PCR reaction using primer set SEQ ID NO: 50 and SEQ ID NO: 51, resulting in an SGIC (SEQ ID NO: 52) comprising flanks at both the 5′ and 3′ end with sequence identity with genomic DNA sequences to allow integration via homologous recombination at the INT1 locus in the genome. An overview of the PCR reactions performed to obtain the SGIC and split SGIC DNA fragments that were used in transformation is presented in Table 5. PCR reactions were performed using PrimeStar GXL DNA polymerase (Takara/Catno. R050A) according to supplier's instructions and a PCR program known to a person skilled in the art.

TABLE 5
Overview of the PCR reactions performed to obtain the split SGIC DNA fragments
and SGIC sequences. The combination of primer sets and template used in
the PCR reaction and resulting SGIC fragment are displayed.

| | | Primers used to | | |
|---|---|---|---|---|
| | | obtain (split) | Sequence of | |
| | Template | SGIC DNA | SGIC DNA | |
| Target | SGIC | fragments | fragment | Make-up of construct |
| INT 1 site, 1 | SEQ ID NO: 4 | SEQ ID NO: 45 | SEQ ID NO: 47 | Connector 5 - SGIC |
| kb deletion | | SEQ ID NO: 46 | | guide-RNA cassette - |
| | | | | connector 3. |
| INT 1 site, 1 | SEQ ID NO: 47 | SEQ ID NO: 48 | SEQ ID NO: 53 | 5′ split SGIC DNA |
| kb deletion | | SEQ ID NO: 50 | | fragment (SGIC part 1) |
| INT 1 site, 1 | SEQ ID NO: 47 | SEQ ID NO: 49 | SEQ ID NO: 54 | 3′ split SGIC DNA |
| kb deletion | | SEQ ID NO: 51 | | fragment (SGIC part 2) |
| INT 1 site, 1 | SEQ ID NO: 47 | SEQ ID NO: 50 | SEQ ID NO: 52 | gDNA-Con5-SGIC |
| kb deletion | | SEQ ID NO: 51 | | guide-RNA cassette - |
| | | | | Con3-gDNA |

The sequences of the resulting split SGIC fragments and (non-split) SGIC flanked by connector sequences and/or genomic DNA sequences (50 bp) for correct integration at the INT1 locus are set out in SEQ ID NO's: 47, 52, 53 and 54. The SGIC consisted of the SNR52p RNA polymerase III promoter, guide-sequence (also referred to as genomic target sequence; SEQ ID NO: 7), the gRNA structural component and the SUP4 3′ flanking region as described in DiCarlo et al., 2013. The 5′ split SGIC fragment consisted of the SNR52p RNA polymerase III promoter, guide-sequence and 30 bp of the guide-RNA structural element for assembly with the 3′ SGIC fragment. The 3′ SGIC fragment consisted of 30 bp of the SNR52p RNA polymerase III promoter, guide-sequence, guide-RNA structural element and SUP4 3′ flanking region. All split SGIC's and non-split SGIC's are depicted in FIGS. 4A-4C.

When no genomic flanks are comprised in the SGIC, 100 bp ssODN's are used for targeted integration on the INT1 locus, SEQ ID NO's: 55, 56, 57 and 58. An overview of the performed transformations and used DNA elements is provided in FIGS. 4A-4C.

### Yeast Transformation Experiments

Strain CSN001 which is pre-expressing Cas9, was transformed using the LiAc/salmon sperm (SS) carrier DNA/PEG method (Gietz and Woods, 2002). An overview of all transformation experiments of Example 2 is provided in Table 5 and Table 6. The experimental set ups are depicted in FIGS. 4A, 4B and 4C.

In each transformation experiment, the SGIC and split SGIC DNA fragments were co-transformed with 50 ng pRN1120, SEQ ID NO:3, and 1 μg of the SGIC DNA fragment (transformation 4B and 4C) or 500 ng of each split SGIC DNA fragment (total 2×500 ng, transformation 4A). In transformation 4B, ssODN flank sequences were included in the transformation, each 50 ng (total: 4×50 ng). In each transformation pRN1120 plasmid (50 ng) was taken along for selection of transformants (Nourseothricin resistance)

The transformation mixtures were plated on YPD-agar (10 grams per liter of yeast extract, 20 grams per liter of peptone, 20 grams per liter of dextrose, 20 gram per liter of agar) containing 200 μg nourseothricin (NTC, Jena BioScience, Germany) and 200 μg G418 (Sigma Aldrich, Zwijndrecht, the Netherlands) per ml.

TABLE 6
Overview of the SGIC DNA's used in different transformations.

| Transformation | | SGIC DNA | ssODN flank |
|---|---|---|---|
| (FIG.) | Description | sequence | sequence |
| 4A | Split SGIC | SEQ ID NO: 53 | |
| | | SEQ ID NO: 54 | |
| 4B | SGIC with | SEQ ID NO: 47 | SEQ ID NO: 55 |
| | separate ssODN | | SEQ ID NO: 56 |
| | flanks | | SEQ ID NO: 57 |
| | | | SEQ ID NO: 58 |
| 4C | SGIC DNA | SEQ ID NO: 52 | |
| | with flanks | | |
| | attached | | |

### Results

The transformation experiment outlined above in Table 6 was performed and after transformation, the cells were placed on YPD selective plates. To confirm correct assembly (transformation 5A) and/or integration of the SGIC type guide-RNA expression cassette (transformation 5A, 5B and 5C) on the INT1 locus, 15 transformants of each transformation were further analyzed by PCR. Genomic DNA of the transformants was isolated as described by Löoke et al., 2011 and was used as template in the PCR reactions. The primers used to confirm the integration were designed to hybridize in the genome just outside the genomic flanking regions that are present in the SGIC DNA (SEQ ID NO: 35 and SEQ ID NO: 36). PCR reactions were performed using MyTag™ Red Mix (Catno BIO-25044, Bioline—Germany) according to manufacturer's instructions and a standard PCR program known to the person skilled in the art.

When using this primer set, correct integration of the SGIC was demonstrated by a PCR product size of 663 bp. In case the SGIC cassette was not integrated on the INT locus a PCR product of 1342 bp was amplified. Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCR products. Results of the PCR analysis of the transformants are displayed in Table 7. In all cases, the PCR analysis resulted in a PCR product: when no SGIC was integrated on the INT1 locus, a product of 1342 bp was amplified. Integration of the SGIC on the INT1 locus results in the amplification of a 663 bp product. A negative PCT result was not taken into account when calculating the success rate of the transformation.

TABLE 7
Overview of the PCR analysis results of SGIC and split SGIC transformants obtained.

| | | | Number of | Number of | |
|---|---|---|---|---|---|
| | Number of | Number of | transformants | transformants | |
| | transformants | positive | with integrated | without integrated | Percentage |
| Transformation | tested | PCR reactions | SGIC | SGIC | edited cells |
| 4A | 15 | 9 | 2 | 7 | 13% |
| 4B | 15 | 13 | 6 | 7 | 46% |
| 4C | 15 | 14 | 8 | 6 | 57% |

The PCR results confirm successful integration of the SGIC type guide-RNA expression cassette in each transformation of Example 2. The transformation of the SGIC with flanks of genomic DNA attached at the 5′ and 3′ end (SEQ ID NO:52) is most successful (57%) of the 3 transformations.

### Example 3: SGIC in Aspergillus niger

SGIC in Aspergillus niger using an SGIC type guide-RNA expression cassette with or without selectable marker cassette and one or two separate fragments as SGIC DNA.

This example describes the disruption of the fnwA locus in genomic DNA of A. niger using Cas9 in combination with an SGIC prepared as a PCR product containing a guide-RNA expression cassette that serves as donor DNA, in absence or presence of an additional selectable marker cassette. By expression (thus before integration) of the guide-RNA, Cas9 is directed to the target site and is able to induce a double strand break at the target site. An overview of the technique is given in FIGS. 9A-9C.

A first approach uses a functional SGIC prepared as a PCR product comprising the guide-RNA expression cassette and 50 bp flanks with homology to genomic DNA at the 5′ and 3′ end, to direct the SGIC to genomic DNA at the intended target site (SGIC fragment I, FIG. 9A). A second approach uses a functional SGIC prepared as PCR product comprising the guide-RNA expression cassette and a marker cassette, that contains 50 bp flanks with homology to genomic DNA at the 5′ and 3′ end, to direct the SGIC to genomic DNA at the intended target site (SGIC fragment II A or SGIC fragment II B. FIG. 9B). A third approach uses a split SGIC comprised of two PCR products: SGIC fragment III comprising the sgRNA expression cassette containing a 50 bp flank with homology to genomic DNA at the 5′ end and a 50 bp flank with homology to SGIC fragment IV A or SGIC fragment IV B at the 3′ end. SGIC fragment IV A and SGIC fragment IV B were prepared by PCR and comprise a marker cassette and contain a 50 bp flank with homology to fragment III at the 5′ end and a 50 bp flank with homology to genomic DNA at the 3′ end (FIG. 9C). Upon transformation, the SGIC fragments form a functional SGIC resulting in disruption of the fwnA gene. Strains with the SGIC (with or without a marker cassette) integrated in the fwnA gene have a color change of the spores from black to fawn (Jorgensen et al., 2011).

### Construction of SGIC DNA Parts

In order to obtain the SGIC DNA fragments depicted in FIGS. 9A-9C and outlined in Table 9, first three DNA parts that contain the fnwA guide-RNA expression cassette and hygromycin or phleomycin marker cassettes were obtained, referred hereafter as SGIC DNA parts. SGIC DNA parts were used as template in a subsequent PCR to obtain SGIC DNA PCR products. For the construction of the three SGIC DNA parts, PCR amplification was performed using Phusion DNA polymerase (New England Biolabs) with primers and template DNA as set out in Table 8, using a standard PCR protocol. All PCR products have Golden-Gate cloning compatible sites. The PCR products were purified with a PCR purification kit from Macherey Nagel (distributed by Bioké, Leiden, The Netherlands) according to manufacturer's instructions. The DNA concentration was measured using a NanoDrop (ND-1000 Spectrophotometer, Thermo Fisher Scientific).

TABLE 8
Overview of the used primers and template to obtain SGIC DNA parts.

| | | | | Resulting TOPO |
|---|---|---|---|---|
| SGIC DNA parts | Forward primer | Reverse primer | Template | vector |
| SGIC DNA part 5′ | SEQ ID NO: 61 | SEQ ID NO: 62 | BG-AMA9 | SEQ ID NO: 76 |
| fwnA flank-sgRNA- | | | | |
| 3′ conH | | | | |
| SGIC DNA hygB | SEQ ID NO: 63 | SEQ ID NO: 64 | BG-AMA9 | SEQ ID NO: 77 |
| marker-3′ fnwA | | | | |
| flank | | | | |
| SGIC DNA phleo | SEQ ID NO: 63 | SEQ ID NO: 64 | BG-AMA5 | SEQ ID NO: 78 |
| marker-3′ fnwA | | | | |
| flank | | | | |

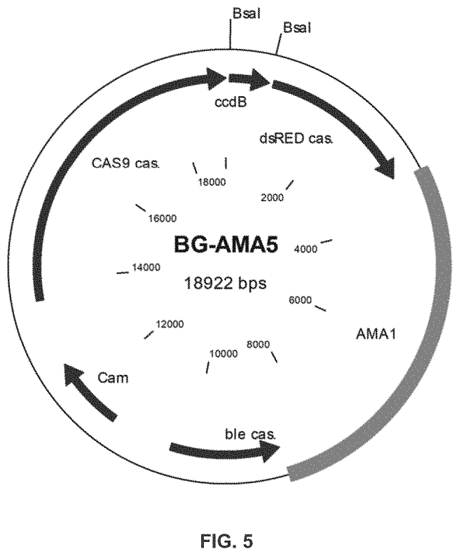

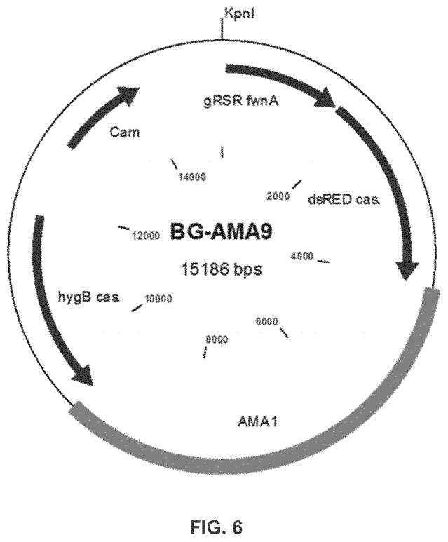

Construction of BG-AMA5 (SEQ ID NO: 65; FIG. 5) and BG-AMA9 (SEQ ID NO: 66; FIG. 6) are described in WO2016110453A1.

The amplified SGIC DNA parts were cloned into a TOPO Zero Blunt vector using the Zero Blunt TOPO PCR Cloning Kit of Invitrogen (SEQ ID NO: 67). The resulting vectors are called “TOPO SGIC DNA sgRNA fwnA”, “TOPO SGIC hygB” and “TOPO SGIC phleo”.

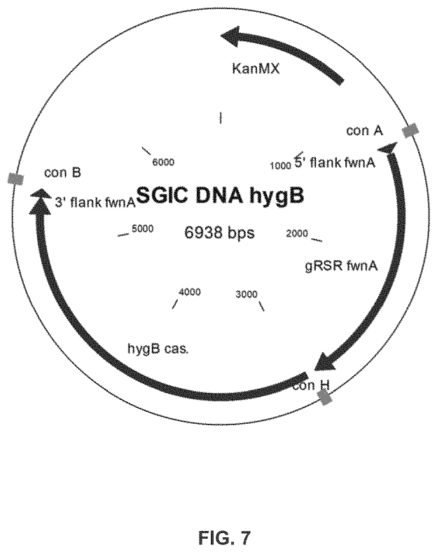

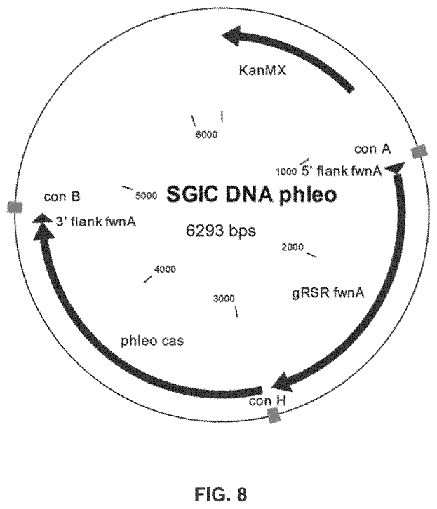

From the TOPO vectors depicted here above, the SGIC DNA parts were transferred using Golden Gate reactions (according to Example 1 in patent application WO2013/144257) into receiving backbone vector AB (SEQ ID: 68). This resulted in the vectors named SGIC DNA HygB” (SEQ ID NO: 69; FIG. 7) and “SGIC DNA Phleo” (SEQ ID NO: 70; FIG. 8).

SGIC DNA Fragments Used in Transformation to A. niger

PCR preparation of SGIC DNA fragments was performed using Phusion DNA polymerase (New England Biolabs) with primers and template DNA as set out in Table 9, using a standard PCR protocol. The PCR products were purified by gel extraction (SGIC fragment I) and by PCR purification (SGIC fragments IIA, IIB, III, IVA and IVB with the Gel and PCR clean up kit from Macherey Nagel (distributed by Bioké, Leiden, The Netherlands) according to manufacturer's instructions. The DNA concentration was measured using a NanoDrop (ND-1000 Spectrophotometer, Thermo Fisher Scientific).

TABLE 9
Overview of the used primers and template to obtain SGIC
DNA fragments used in transformations to A. niger.

| | SGIC fragment | | | | |
|---|---|---|---|---|---|
| SGIC DNA | name | Forward primer | Reverse primer | Template | Resulting sequence |
| FwnA sgRNA/ | I | SEQ ID NO: 72 | SEQ ID NO: 71 | SGIC DNA | SEQ ID NO: 85 |
| 5′_3′ flank | | | | HygB | |
| | | | | SGIC DNA | |
| | | | | phleo | |
| FwnA sgRNA/hygB/ | II A | SEQ ID NO: 72 | SEQ ID NO: 73 | SGIC DNA | SEQ ID NO: 86 |
| 5′_3′ flank | | | | HygB | |
| FwnA sgRNA/phleo/ | II B | SEQ ID NO: 72 | SEQ ID NO: 73 | SGIC DNA | SEQ ID NO: 87 |
| 5′_3′ flank | | | | Phleo | |
| FwnA sgRNA/ | III | SEQ ID NO: 72 | SEQ ID NO: 74 | SGIC DNA | SEQ ID NO: 88 |
| 5′_conH flank | | | | HygB | |
| hygB/conH_3′ flank | IV A | SEQ ID NO: 75 | SEQ ID NO: 73 | SGIC DNA | SEQ ID NO: 89 |
| | | | | HygB | |
| phleo/conH_3′ flank | IV B | SEQ ID NO: 75 | SEQ ID NO: 73 | SGIC DNA | SEQ ID NO: 90 |
| | | | | Phleo | |

FIGS. 9A-9C provide a graphical representation of the approaches to integrate the fwnA SGIC with/without separate marker cassette into the genome of A. niger at the fnwA locus.

### Construction of BG-AMA17 Plasmid

PCR amplification of the Cas9 expression cassette (construction of BG-C20 Cas9 expression cassette is described in WO2016110453A1) was performed using Phusion DNA polymerase (New England Biolabs), and forward primer as set out in SEQ ID NO: 79 and reverse primer as set out in SEQ ID NO: 80. Both primers contained flanks with a KpnI restriction site. The PCR products were purified with a PCR purification kit from Macherey Nagel (distributed by Bioké, Leiden, the Netherlands) according to manufacturer's instructions. The DNA concentration was measured using a NanoDrop (ND-1000 Spectrophotometer, Thermo Fisher Scientific).

Backbone vector BG-AMA8 (described in WO2016110453A1) and the obtained KpnI flanked PCR fragment of the Cas9 expression cassette were digested with KpnI (NEB-enzymes) and purified with a PCR purification kit from Macherey Nagel (distributed by Bloke, Leiden, The Netherlands). Digested BG-AMA8 backbone vector and Cas9 cassette PCR product were ligated with T4 ligation (Invitrogen) according to manufacturer's instructions. The ligation mix was transformed to ccdB resistant E. coli cells (Invitrogen) according to manufacturer's instructions. Several clones were checked with restriction enzyme analysis and a clone having the correct restriction pattern was named BG-AMA17 (SEQ ID NO: 83). A plasmid map of BG-AMA17 is provided in FIG. 13. Plasmid BG-AMA17 contains a Cas9 expression cassette expressed from a promoter and terminator, a dsRED cassette and a HygB marker for selection in A. niger.

### Strain

In this example, Aspergillus niger strain GBA 302 (ΔglaA, ΔpepA, ΔhdfA) was used in the transformation experiments. The construction of GBA 302 is described in patent application WO2011/009700.

### Transformation

Protoplast transformation was performed as described in patent applications WO1999/32617 and WO1998/46772, except for the addition of ATA (Aurintricarboxylic acid=nuclease inhibitor) in the transformation mixture. In these transformations, Cas9 protein containing a nuclear localization signal (NLS) was used (IDT, Integrated DNA Technologies, Inc). The Cas9 used in this example was either expressed from an AMA-vector depicted here above or was added as Cas9 protein to the transformation. 50 μg of the Cas9 protein was dissolved in 50 μl nuclease free water (Ambion, Thermo Fisher, Bleiswijk, The Netherlands) to a final concentration of 1 μg/μl. 1.5 μg of Cas9 protein was used in the respective transformations.

### Experimental Design SGIC Experiments and Resulting Data

Tables 10-15 describe six sub-sets of SGIC experiments. These tables all have the same column captions. The columns “AMA” indicates whether an AMA vector was added in the transformation, with “x” indicating no AMA plasmid; “phleo” indicating addition of an AMA plasmid with a phleo marker cassette (BG-AMA1, FIG. 14, SEQ ID NO: 84) and “hygB” indicating an AMA plasmid with a hygB marker cassette (BG-AMA8, FIG. 11, SEQ ID NO: 81). The columns “Cas9” indicates how the Cas9 protein is provided to the cells: “x” means “no Cas9”, “protein” means added as protein in the transformation mix, “Cas9 st” means that Cas9 is encoded at the AMA plasmid and expressed from the strong promoter Pc_FP017.pro with the Pc_FT029.terminator (with phleo marker=BG-AMA5, SEQ ID: 65, FIG. 5; with hygB marker=BG-AMA17, SEQ ID: 83, FIG. 13), “Cas9++” means that Cas9 is encoded at the AMA plasmid and expressed from the very strong promoter A. nidulans TEF.pro with the Pc_FT029.ter (BG-AMA14, SEQ ID: 82, FIG. 10). The column “selection” indicates for which marker is being selected on the transformation plates: “phleo” indicates selection on phleomycin and hygB indicates selection on hygromycin B.

First, two series of SGIC experiments were performed according to the concept shown in FIG. 9A (transformation of SGIC fragment I) and further explained in Tables 10 and 11. Tables 10 and 11 are schematically depicted in detail in FIGS. 12A-12G, where rows A, B, C, D, E, F, G are represented by the respective FIGS. 12A-12G. In case of Table 10, no SGIC is supplied as a control and for the experiment in Table 11, the SGIC fragment (SEQ ID NO: 85 [SGIC fragment I]) is supplied as visualized in the table.

Second, two series of SGIC experiments were performed according to the concept shown in FIG. 9B (transformation of SGIC fragment II A and SGIC fragment IIB) and further described in Tables 12 and 13, respectively.

Third, two series of SGIC experiments were performed according the concept shown in FIG. 9C (transformation of two split SGIC fragments III+IVA, and SGIC fragment III+IVB) and further described in Tables 14 and 15, respectively.

TABLE 10
No SGIC fragment used

| Row | AMA | CAS9 | selection | # colonies | # fawn | % fawn |
|---|---|---|---|---|---|---|
| A | phleo | x | phleo | 40 | 0 | 0 |
| B | phleo | protein | phleo | 15 | 0 | 0 |
| C | phleo | Cas9 st | phleo | 31 | 0 | 0 |
| D | phleo | Cas9++ | phleo | 0 | 0 | 0 |
| E | hygB | x | hygB | 120 | 0 | 0 |
| F | hygB | protein | hygB | 31 | 0 | 0 |
| G | hygB | Cas9 st | hygB | 62 | 0 | 0 |

Table 10 provides the results of the control experiments without the addition of SGIC DNA. All spores obtained in experiments A-G show the black phenotype. This means that no editing of the fwnA locus took place. Note that 0 colonies where obtained in case of using a very-strong promoter for Cas9 at the AMA plasmid (row 10D), indicating that a high availability of Cas9 is hampering cell growth or recovery after transformation.

TABLE 11
SGIC used: SEQ ID NO: 85 [SGIC fragment I]

| Row | AMA | CAS9 | selection | # colonies | # fawn | % fawn |
|---|---|---|---|---|---|---|
| A | phleo | x | phleo | 91 | 0 | 0 |
| B | phleo | protein | phleo | 171 | 0 | 0 |
| C | phleo | Cas9 st | phleo | 50 | 31 | 62 |
| D | phleo | Cas9++ | phleo | 12 | 1 | 8 |
| E | hygB | x | hygB | >500 | 0 | 0 |
| F | hygB | protein | hygB | >500 | 2 | 0.4 |
| G | hygB | Cas9 st | hygB | 416 | 151 | 36 |

By repeating the experiment with the addition of a SGIC DNA targeting the fawn locus (SEQ ID NO: 85 [SGIC fragment I]), we clearly observed fawn colonies in all cases where Cas9 is available to the cells, except for transformation 11B The frequency of targeted insertion of the SGIC is between 0.4 and 62%, depending on the marker present on the AMA vector and on the expression strength of the promoter used for Cas9 expression or direct use of a Cas9 protein. In all positive editing cases, the selection marker cassette was present at the AMA plasmid, not on the SGIC.

Next we performed an experiment with a SGIC that contains a selectable marker (FIG. 5B), this in with variation in the selection on the applied AMA vector and/or SGIC construct, Table 12.

TABLE 12
SGIC used. SEQ ID: 86 [SGIC fragment II A, HygB marker part
of SGIC DNA]

| Row | AMA | CAS9 | selection | # colonies | # fawn | % fawn |
|---|---|---|---|---|---|---|
| A | x | X | hygB | 0 | 0 | 0 |
| B | x | Protein | hygB | 88 | 79 | 90 |
| C | hygB | Protein | hygB | >400 | 7 | 2 |
| D | hygB | Cas9 st | hygB | 305 | 224 | 73 |
| E | phleo | Protein | phleo | 75 | 0 | 0 |
| F | phleo | Cas9 st | phleo | 68 | 36 | 53 |
| G | phleo | Cas9++ | phleo | 0 | 0 | 0 |
| H | phleo | Cas9 st | hygB | 370 | 351 | 95 |
| I | phleo | Cas9++ | hygB | 287 | 280 | 98 |

The results from Table 12 show very high efficiencies for the introduction of the SGIC fragment at the fnwA locus, reaching up to 98%. Note that the system without AMA vector, row 12B, gives a high number of transformants with a 90% editing efficiency, while the control 12A gives no colonies, demonstrating that in the absence of Cas9 the SGIC fragment is not integrated into genomic DNA. This set of experiments clearly shows that the SGIC concept with transient expression of a sgRNA from a linear double stranded SGIC DNA allows for efficient introduction—in this case the SGIC fragment itself containing the sgRNA expression cassette and a hygB expression cassette—into the genome, facilitated by the Cas9 double stranded genomic DNA cleavage. Highest editing efficiencies were obtained when selecting for the hygB at the SGIC DNA where the AMA contains a different marker (here phleo) (integration of the SGIC in genomic DNA, 12B, H, I); editing efficiencies were lower when the hygB marker is also available at the AMA plasmid (12C, D).

TABLE 13
SGIC used: SEQ ID NO: 87 [SGIC fragment II B, phleo marker
part of SGIC DNA]

| Row | AMA | CAS9 | Selection | # colonies | # fawn | % fawn |
|---|---|---|---|---|---|---|
| A | x | x | phleo | 0 | 0 | 0 |
| B | x | protein | phleo | 9 | 9 | 100 |
| C | phleo | protein | phleo | >400 | 8 | 2 |
| D | phleo | Cas9 st | phleo | 192 | 122 | 64 |
| E | phleo | Cas9++ | phleo | 246 | 208 | 85 |
| F | hygB | protein | hygB | >400 | 29 | 7 |
| G | hygB | Cas9 st | hygB | 136 | 55 | 40 |

Table 13 provides the results of a similar experiment as in Table 12, but now with a phleomycine marker present on the SGIC construct. Similar to 12B, here the Cas9 protein transformation with selection for the marker at the SGIC also provided highest editing efficiency, with an editing to efficiency of 100% (Table 13 row B).

Next, two experimental sets were made (Table 14 and Table 15), where the SGIC DNA is formed in the cell via homologous recombination of two SGIC fragments (split SGIC), namely a first fragment containing a sgRNA expression cassette, and a second fragment containing a marker cassette (FIG. 5C).

TABLE 14
SGIC fragments used: SEQ ID: 88 (SGIC fragment III) + SEQ ID: 89
[SGIC fragment IV A, HygB marker part of SGIC DNA]

| Row | AMA | CAS9 | Selection | # colonies | # fawn | % fawn |
|---|---|---|---|---|---|---|
| A | x | x | hygB | 0 | 0 | 0 |
| B | x | protein | hygB | 49 | 39 | 80 |
| C | hygB | protein | hygB | >500 | 0 | 0 |
| D | hygB | Cas9 st | hygB | 303 | 150 | 50 |
| E | phleo | protein | phleo | 213 | 0 | 0 |
| F | phleo | Cas9 st | phleo | 21 | 9 | 43 |
| G | phleo | Cas9++ | phleo | 0 | 0 | 0 |

The results of Table 14 A-G can be directly compared to those of Table 12 A-G, where the only difference between both is the use of 1 versus 2 fragments to constitute a functional SGIC. Overall, both tables provide a rather consistent view with highest frequency of editing when selecting only for the marker (hygB) present at the SGIC DNA. It can be concluded that SGIC fragment can be formed efficiently via homologous recombination, and thus can be provided as two fragments (split SGIC).

TABLE 15
SGIC fragments used: SEQ ID: 88 (SGIC fragment III) + SEQ ID: 90
[SGIC fragment IV B, phleo marker part of SGIC DNA]

| Row | AMA | CAS9 | Selection | # colonies | # fawn | % fawn |
|---|---|---|---|---|---|---|
| A | x | X | phleo | 22 | 0 | 0 |
| B | x | Protein | phleo | 34 | 26 | 76 |
| C | phleo | Protein | phleo | >300 | 0 | 0 |
| D | phleo | Cas9 st | phleo | 186 | 89 | 48 |
| E | phleo | Cas9++ | phleo | 75 | 67 | 89 |
| F | hygB | Protein | hygB | >500 | 0 | 0 |
| G | hygB | Cas9 st | hygB | 104 | 28 | 27 |

The results of Table 15 A-G can be compared directly with those of Table 13 A-G, where the only difference between both is the use of 1 versus 2 fragments to constitute a functional SGIC. Overall, both tables provide a rather consistent view with highest frequency of editing when selecting only for the marker (phleo) at the SGIC DNA. It can be concluded that SGIC fragment can be formed efficiently via homologous recombination, and thus can be provided as two fragments (split SGIC).

### Example 4: Multiplex Genome Editing by SGIC in S. cerevisiae

This example describes integration of multiple Self-Guiding Integration Constructs (SGICs) type guide-RNA expression cassettes using a CRISPR/Cas9 system in Saccharomyces cerevisiae. The SGIC's comprised 50 bp flanks at both the 5′ and 3′ end with sequence identity with genomic DNA sequences to allow integration via homologous recombination at the desired genomic locus. Depending on the sequence of the flanks, a stretch of DNA of up to 1 kbp was deleted from the genome upon integration of the SGIC. When the flank sequences were homologous to a sequence surrounding an ORF, upstream of the ATG start codon and downstream of the STOP codon, a complete ORF was deleted. This set-up is visually shown in FIG. 15L.

In the SGICs, for the expression of guide-RNA's in S. cerevisiae, a guide-RNA expression cassette with control elements as previously described by DiCarlo et al., 2013 was used. The guide-RNA expression cassettes used in this example comprised the SNR52 promoter, a guide-RNA sequence consisting of the guide-sequence (also referred to as genomic target sequence) and the guide-RNA structural component followed by the SUP4 terminator.

### Experimental Details

The components applied in this example were as follows:
Yeast strain CSN001 which is pre-expressing Cas9. Construction of the strain CSN001 is described in Example 1.
pRN1120, multi-copy expression vector containing NatMX marker. Construction and details of the plasmid are described in Example 1.

### Self-Guiding Integration Construct (SGIC) Type Guide-RNA Expression Cassettes

Synthetic DNAs containing guide-RNA expression cassettes (SEQ ID NO. 91, 92 and 93) were ordered as synthetic DNA (gBlocks) at Integrated DNA Technologies (IDT, Leuven, Belgium). The gBlock DNAs were used as template in a PCR reaction, using primers as indicated in Table 16, and using PrimeSTAR GXL DNA Polymerase (Takara/Cat no. R050A) according to the manufacturer's instructions. The resulting SGIC DNAs, of which the sequences are set out in SEQ ID NOs: 103, 104 and 105, consisted of the SNR52p RNA polymerase III promoter, a guide-sequence (also referred to as genomic target sequence; SEQ ID NOs: 94, 95, 96), the gRNA structural component and the SUP4 3′ flanking region as described in DiCarlo et al., 2013, and include a 50 bp genomic DNA sequence at both the 5′ and 3′ end for integration at the genomic locus. An overview of the sequences is provided in Table 16. The 50 bp genomic sequence at the 5′ and 3′ end of SGIC is identical to the genomic sequence just outside an ORF, upstream of the ATG, start codon, and downstream of the STOP codon. This means the ORF that is targeted by the guide-RNA expression cassette of the SGIC DNA is deleted upon integration of the SGIC DNA. The size of the complete ORF that is deleted by integration of the SGIC DNAs (SEQ ID NO: 103, 104 and 105), is 2376 bps, 1308 bps and 651 bps, respectively for ORF1, ORF2 and ORF3. The generated SGIC DNA's were purified using a NucleoSpin Gel and PCR Clean-up kit (Machery-Nagel, distributed by Bioké, Leiden, the Netherlands) according to manufacturer's instructions. Subsequently, DNA concentrations of purified SGIC DNA's were measured using a NanoDrop (ND-1000 Spectrophotometer, Thermo Scientific, Bleiswijk, the Netherlands).

TABLE 16
Overview of the sequences of the SGIC DNA's used in transformation.
The template guide-RNA expression cassettes were used as a template for
PCR using the primers indicated in this table in order to obtain SGIC
DNA's (SGIC DNA fragments) used in the transformation experiments.

| | Template guide- | Guide sequence | | Sequence of |
|---|---|---|---|---|
| | RNA expression | (genomic target | Primers used to | the SGIC DNA |
| Target | cassette | sequence) | obtain SGIC DNA | fragment |
| ORF1 | SEQ ID NO: 91 | SEQ ID NO: 94 | SEQ ID NO: 97 | SEQ ID NO: 103 |
| (YER109C) | | | SEQ ID NO: 98 | |
| ORF2 | SEQ ID NO: 92 | SEQ ID NO: 95 | SEQ ID NO: 99 | SEQ ID NO: 104 |
| (YML051W) | | | SEQ ID NO: 100 | |
| ORF3 | SEQ ID NO: 93 | SEQ ID NO: 96 | SEQ ID NO: 101 | SEQ ID NO: 105 |
| (YHR128W) | | | SEQ ID NO: 102 | |

### Yeast Transformation

Strain CSN001 which is pre-expressing Cas9, was inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN001 was transformed with 1 μg of SGIC DNA as indicated in Table 17, using the LiAc/SS carrier DNA/PEG method (Gietz and Woods, 2002) and 100 ng vector pRN1120. In transformation #4 no SGIC DNA was added to the transformation mixture. The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 17
Overview of SGIC DNA's used in the
multiplex transformation experiments

| | | | Amount of |
|---|---|---|---|
| Transformation | Target | SGIC DNA | SGIC DNA |
| #1 | ORF1 | SEQ ID NO: 103 | 1000 ng |
| #2 | ORF1 | SEQ ID NO: 103 | 500 ng |
| | ORF2 | SEQ ID NO: 104 | 500 ng |
| #3 | ORF1 | SEQ ID NO: 103 | 350 ng |
| | ORF2 | SEQ ID NO: 104 | 350 ng |
| | ORF3 | SEQ ID NO: 105 | 350 ng |
| #4 | No SGIC DNA added | — | — |
| | (control) | | |

### Results

The transformation experiment outlined above in Table 17 was performed and after transformation, the cells were plated on YPD selective plates. To confirm correct integration of the SGIC comprising the guide-RNA expression cassette and to deletion of the targeted ORF in the genome, 8 transformants were analyzed by PCR. Genomic DNA of the transformants was isolated as described by Löoke et al., 2011 and was used as template in a PCR reaction.

The first primer of the primer set used to confirm the integration was designed to hybridize to the genome just outside the genomic flanking regions that are present in the SGIC DNA. The second primer of the primer set was designed to hybridize the guide-RNA expression cassette of the SGIC DNA construct. PCR reactions were performed using MyTag™ Red Mix (Cat.no. BIO-25044, Bioline—Germany) according to manufacturer's instructions and a standard PCR program known to the person skilled in the art. When using the primer sets that are set out in Table 18 in the PCR reaction, correct integration was demonstrated by a PCR product of the size as mentioned in the most right column of Table 18. Resulting PCR products were analyzed on a 0.8% agarose gel using 1×TAE buffer (50×TAE (Tris/Acetic Acid/EDTA), 1 liter, Cat no. 1610743, BioRad, The Netherlands) and 520-Nancy (Cat no. 01494, Sigma Aldrich, Germany) to stain the PCR products.

TABLE 18
Overview of analysis of transformants by PCR

| | | | Product size: | | Product size: |
|---|---|---|---|---|---|
| | | | no SGIC | | SGIC |
| Transformation | Target | Primer set | integration | | integration |
| #1 | ORF1 | SEQ ID NO: 106 | 1852 | bp | 258 bp |
| | | SEQ ID NO: 107 | | | |
| # 2 | ORF1 | SEQ ID NO: 106 | 1852 | bp | 258 bp |
| | | SEQ ID NO: 107 | | | |
| | ORF2 | SEQ ID NO: 108 | 730 | bp | 270 bp |
| | | SEQ ID NO: 109 | | | |
| # 3 | ORF1 | SEQ ID NO: 106 | 1852 | bp | 258 bp |
| | | SEQ ID NO: 107 | | | |
| | ORF2 | SEQ ID NO: 108 | 730 | bp | 270 bp |
| | | SEQ ID NO: 109 | | | |

| | | | |
|---|---|---|---|
| ORF3 | SEQ ID NO: 110 | no fragment | 241 bp |
| | SEQ ID NO: 111 | (by design) | |

The transformation of plasmid pRN1120 without addition of SGIC DNA, transformation #4, was performed to check the transformation efficiency of strain CSN001, no transformants of this transformation were further analyzed.

An overview of the results of the PCR reactions performed to analyze transformants for correct integration of SGIC DNA is displayed here below in Table 19.

TABLE 19
Overview of the results of the colony PCR performed to confirm integration of the SGIC comprising
the guide-RNA expression cassette at the correct location in the genome. When a transformation
is performed with multiple SGIC DNA constructs and the result is mentioned as 1x SGIC: 2, it
means that out of the 8 transformants that were screened, there were 2 transformants that contain
integration of either one of the SGIC DNA constructs used in transformation.

| | | | | Number of | |
|---|---|---|---|---|---|
| | | Number of | | transformants | |
| | Total number of | transformants | | with and without | Percentage of |
| Transformation | transformants | characterized | Target (s) | integrated SGIC | edited cells |
| # 1 | 320 | 8 | ORF1 | 0x SGIC: 1 | 12.5% |
| | | | | 1x SGIC: 7 | 87.5% |
| # 2 | 61 | 8 | ORF1 | 0x SGIC: 1 | 12.5% |
| | | | ORF2 | 1x SGIC: 2 | 25% |
| | | | | 2x SGIC: 5 | 62.5% |
| # 3 | 65 | 8 | ORF1 | 0x SGIC: 5 | 62.5% |
| | | | ORF2 | 1x SGIC: 2 | 25% |
| | | | ORF3 | 2x SGIC: 0 | 0% |
| | | | | 3x SGIC: 1 | 12.5% |

In this experiment, it is confirmed by the limited screening of only 8 transformants per transformation, that it is possible to create multiple knock-out mutants in one transformation by addition of multiple SGIC DNA constructs in Saccharomyces cerevisiae.

This successful experiment indicates that the invented method allows for rapid modular multiplexing of SGIC constructs. In this example the transformants with integrated SGIC constructs are characterized via PCR. In practice, this could also be done via whole genome NGS sequencing or targeted sequencing of the unique sequences of the SGIC inserts, e.g. the guide sequence or an added DNA barcode within the SGIC construct.

### REFERENCES

Altschul S F et al., J. Mol. Biol. 215:403-410 (1990)
Carillo H and Lipman D. SIAM J. Applied Math., 48:1073 (1988)
Carrel F. L. Y. and Canevascini G. Canadian Journal of Microbiology (1991) 37(6): 459-464; Reese E. T., Parrish F. W. and Ettlinger M. Carbohydrate Research (1971) 381-388.
Chaveroche, M K., Ghico, J-M. and d'Enfert C. A rapid method for efficient gene replacement in the filamentous fungus Aspergillus nidulans (2000); Nucleic acids Research, vol 28, no 22.
Cong L, Ran F A, Cox D, Lin S, Barretto R, Habib N, Hsu P D, Wu X, Jiang W, Marraffini L A, Zhang F. Science. Multiplex genome engineering using CRISPR/Cas systems. 2013 Feb. 15; 339(6121):819-23. doi: 10.1126/science.1231143. Epub 2013 Jan. 3.
Crook N C, Schmitz A C, Alper H S. Optimization of a yeast RNA interference system for controlling gene expression and enabling rapid metabolic engineering. ACS Synth Biol. 2014 May 16; 3(5):307-13.
Devereux, J., et al., Nucleic Acids Research 12 (1): 387 (1984).
Derkx, P M and Madrid S M. The foldase CYPB is a component of the secretory pathway of Aspergillus niger and contains the endoplasmic reticulum retention signal HEEL. Mol. Genet. Genomics. 2001 December; 266(4):537-545
DiCarlo J E, Norville J E, Mali P, Rios X, Aach J, Church G M. Nucleic Acids Res. 2013 April; 41(7):4336-43. Genome engineering in Saccharomyces cerevisiae using CRISPR-Cas systems.
DiCarlo J E, Chavez A, Dietz S L, Esvelt K M, Church G M. Safeguarding CRISPR-Cas9 gene drives in yeast. Nat Biotechnol. 2015 December; 33(12):1250-1255. doi: 10.1038/nbt.3412.
Egholm M, Buchardt O, Christensen L, Behrens C, Freier S M, Driver D A, Berg R H, Kim S K, Norden B, Nielsen PE., 1993. Nature 365, 566-568.
Flagfeldt D B, Siewers V, Huang L, Nielsen J. Characterization of chromosomal integration sites for heterologous gene expression in Saccharomyces cerevisiae. Yeast. 2009 October; 26(10):545-51. doi: 10.1002/yea.1705.
Gao F, Shen X Z, Jiang F, Wu Y, Han C. DNA-guided genome editing using the Natronobacterium gregoryi Argonaute. Nat Biotechnol. 2016 July; 34(7):768-73. doi: 10.1038/nbt.3547.
Gietz R D, Woods R A. Transformation of yeast by lithium acetate/single-stranded carrier DNA/polyethylene glycol method. Methods Enzymol. 2002; 350:87-96.
Govindaraju and Kumar, 2005. Chem. Commun, 495-497.
Gribskov M and Devereux J, eds., Sequence Analysis Primer, M Stockton Press, New York, 1991.
Griffin H M and Griffin H G, eds., Computer Analysis of Sequence Data, Part I, Humana Press, New Jersey, 1994.
Griffin H M and Griffin H G, eds., Molecular Biology: Current Innovations and Future Trends. ISBN 1-898486-01-8; 1995 Horizon Scientific Press, PO Box 1, Wymondham, Norfolk, U.K
Gupta et al. (1968), Proc. Natl. Acad. Sci USA, 60: 1338-1344.
Hawksworth D L et al., In, Ainsworth and Bisby's Dictionary of The Fungi, 8th edition, 1995, CAB International, University Press, Cambridge, UK
Herbert R B. The Biosynthesis of Secondary Metabolites, Chapman and Hall, New York, 1981.
Ho S N, Hunt H D, Horton R M, Pullen J K, Pease L R “Site-directed mutagenesis by overlap extension using the polymerase chain reaction. Gene. 1989 Apr. 15; 77(1):51-9.
Jorgensen T R, Park J, Arentshorst M, van Welzen A M, Lamers G, Vankuyk P A, Damveld R A, van den Hondel C A, Nielsen K F, Frisvad J C, Ram A F. Fungal Genet Biol. 2011 May; 48(5):544-53. The molecular and genetic basis of conidial pigmentation in Aspergillus niger.
Kamath R S et al, (2003) Systematic functional analysis of the Caenorhabditis elegans genome using RNAi. Nature. Vol. 421, 231-237.
Lesk A. M. ed. Computational Molecular Biology, Oxford University Press, New York, 1988.
Löoke M, Kristjuhan K, Kristjuhan A. Biotechniques. 2011 May; 50(5):325-8. Extraction of genomic DNA from yeasts for PCR-based applications.
Mali P, Yang L, Esvelt K M, Aach J, Guell M, DiCarlo J E, Norville J E, Church G M. RNA-guided human genome engineering via Cas9. Science. 2013 Feb. 15; 339(6121):823-6. doi: 10.1126/science.1232033. Epub 2013 Jan. 3.
Maruyana et al. Nat Biotechnol. 2015 May; 33(5): 538-542.
Song et al. Nature communications|doi: 10.1038/ncomms10548
Yu et al. Cell Stem Cell. 2015 February 5; 16(2): 142-147.
Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.
Morita et al. 2001. Nucleic Acid Res Supplement No. 1: 241-242.
Mouyna I, Henry C, Doering T L, Latgé J P. Gene silencing with RNA interference in the human pathogenic fungus Aspergillus fumigatus. FEMS Microbiol Lett. 2004 Aug. 15; 237(2):317-24.
Nakamura Y, Gojobori T, Ikemura T. Codon usage tabulated from international DNA sequence databases: status for the year 2000. Nucleic Acids Res. 2000 Jan. 1; 28(1):292.
Needleman and Wunsch, J. Mol. Biol. 48:443-453 (1970).
Ngiam C, Jeenes D J, Punt P J, Van Den Hondel C A, Archer D B. Appl. Environ. Microbiol. 2000 February; 66(2):775-82. Characterization of a foldase, protein disulfide isomerase A, in the protein secretory pathway of Aspergillus niger.
Nielsen et al., 1991. Science 254, 1497-1500.
Pel et al. Genome sequencing and analysis of the versatile cell factory Aspergillus niger CBS 513.88. Nat Biotechnol. 2007 February; 25 (2):221-231.
Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.
Scarpulla et al. (1982), Anal. Biochem. 121: 356-365.
Sikorski R S, Hieter P. Genetics. A system of shuttle vectors and yeast host strains designed for efficient manipulation of DNA in Saccharomyces cerevisiae. 1989 May; 122(1):19-27.
Smith D W, ed., Biocomputing: Informatics and Genome Projects, Smith, Academic Press, New York, 1993.
Stemmer et al. (1995), Gene 164: 49-53.
Tour O. et al, (2003) Nat. Biotech: Genetically targeted chromophore-assisted light inactivation. Vol. 21. no. 12:1505-1508.
van Dijck et al, 2003, Regulatory Toxicology and Pharmacology 28; 27-35: On the safety of a new generation of DSM Aspergillus niger enzyme production strains.
van Dijken J P, Bauer J, Brambilla L, Duboc P, Francois J M, Gancedo C, Giuseppin M L, Heijnen J J, Hoare M, Lange H C, Madden E A, Niederberger P, Nielsen J, Parrou J L, Petit T, Porro D, Reuss M, van Riel N, Rizzi M, Steensma H Y, Verrips C T, Vindeløv J, Pronk J T. An interlaboratory comparison of physiological and genetic properties of four Saccharomyces cerevisiae strains. Enzyme Microb Technol. 2000 Jun. 1; 26 (9-10):706-714.
Vartak S V and Raghavan S C. Inhibition of nonhomologous end joining to increase the specificity of CRISPR/Cas9 genome editing. FEBS J. 2015 November; 282(22):4289-94. doi: 10.1111/febs.13416. Epub 2015 Sep. 9.
von Heine G. Sequence Analysis in Molecular Biology, Academic Press, 1987.
Young and Dong, (2004), Nucleic Acids Research 32 (7).
Zrenner R, Willmitzer L, Sonnewald U. Analysis of the expression of potato uridinediphosphate-glucose pyrophosphorylase and its inhibition by antisense RNA. Planta. (1993); 190(2):247-52.

1. A self-guiding integration construct comprising:
a guide-RNA expression cassette, and
an additional polynucleotide element,
wherein said guide-RNA expression cassette is capable of expressing a functional guide-RNA, or a part thereof, that is specific for a target sequence in a target genome, wherein the part of the self-guiding integration construct comprising said guide-RNA expression cassette and said additional polynucleotide element is flanked at its 5′-terminus by a first polynucleotide and at its 3′-terminus by a second polynucleotide, and wherein said first and second polynucleotide have sequence identity with sequences flanking the target sequence in the target genome; with the proviso that the self-guiding integration construct does not comprise an expression construct encoding a polynucleotide-guided genome editing enzyme.

2. A self-guiding integration construct comprising:
a guide-RNA expression cassette, and optionally,
a additional polynucleotide element, wherein said guide-RNA expression cassette is capable of expressing a functional guide-RNA, or a part thereof, that is specific for a target sequence in a target genome, wherein the part of the self-guiding integration construct comprising said guide-RNA expression cassette and optionally said additional polynucleotide element is flanked at its 5′-terminus by a first polynucleotide and at its 3′-terminus by a second polynucleotide, wherein said first and second polynucleotide have sequence identity with sequences flanking the target sequence in the target genome, and wherein the functional guide-RNA, or the part thereof, is encoded by a polynucleotide on the guide-RNA expression cassette and said polynucleotide is operably linked to an RNA polymerase II promoter, to an RNA polymerase III promoter as well as a self-processing ribozyme or to a single-subunit DNA-dependent RNA polymerase promoter, optionally a viral single-subunit DNA-dependent RNA polymerase promoter, optionally a T3, SP6, K11 or T7 RNA polymerase promoter; with the proviso that the self-guiding integration construct does not comprise an expression construct encoding a polynucleotide-guided genome editing enzyme.

3. A self-guiding integration construct comprising:
two or more polynucleotides capable of recombining with each other to yield a guide-RNA expression cassette, and optionally, an additional polynucleotide element,
wherein said guide-RNA expression cassette is capable of expressing a functional guide-RNA, or a part thereof, wherein said functional guide-RNA or part thereof is specific for a target sequence in a target genome, wherein the part of the self-guiding integration construct comprising said guide-RNA expression cassette and optionally said additional polynucleotide element is flanked at its 5′-terminus by a first polynucleotide and at its 3′-terminus by a second polynucleotide, and wherein said first and second polynucleotide have sequence identity with sequences flanking the target sequence in the target genome; with the proviso that the self-guiding integration construct does not comprise an expression construct encoding a polynucleotide-guided genome editing enzyme.

4. A self-guiding integration construct according to claim 1, wherein the self-guiding integration construct is a linear self-guiding integration construct.

5. A composition comprising two or more polynucleotide members, wherein said members have sequence identity with each other which allows them to recombine in vivo, optionally in a host cell, to yield a single self-guiding integration construct according to claim 1 optionally to yield a linear self-guiding integration construct.

6. The self-guiding integration construct according to claim 1, wherein the additional polynucleotide element is a control sequence, a marker, a gene of interest, or a disruption construct.

7. A composition comprising a self-guiding integration construct as defined in claim 1, optionally comprising a library of self-guiding integration constructs, said composition optionally further comprising a functional polynucleotide-guided genome editing enzyme and/or an expression construct capable of expressing a functional polynucleotide-guided genome editing enzyme.

8. A host cell comprising a self-guiding integration construct as defined in claim 1.

9. A host cell according to claim 8, further comprising a functional polynucleotide-guided genome editing enzyme, optionally a functional polynucleotide-guided heterologous genome editing enzyme, or further comprising an expression construct capable of expressing a functional polynucleotide-guided genome editing enzyme, optionally a functional polynucleotide-guided heterologous genome editing enzyme.

10. A host cell according to claim 8, wherein the self-guiding integration construct is integrated into the genome at the site where the first and second polynucleotide have sequence identity with the sequences flanking the target sequence in the target genome.

11. A self-guiding integration construct for ex vivo use comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, for expression of a functional guide-RNA or part thereof that is specific for a target sequence in a target genome, in a host cell, wherein the functional guide-RNA, or part thereof that is specific for a target sequence in a target genome, is exclusively expressed from the self-guiding integration construct; with the proviso that the self-guiding integration construct does not comprise an expression construct encoding a polynucleotide-guided genome editing enzyme.

12. A composition for ex vivo use comprising two or more polynucleotide members, wherein these members have sequence identity with each other which allows them to recombine in vivo, optionally in a host cell, to yield a self-guiding integration construct comprising a guide-RNA expression cassette, wherein said guide-RNA expression cassette is capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, for the expression of a functional guide-RNA or part thereof that is specific for a target sequence in a target genome in a host cell, wherein the functional guide-RNA, or part thereof that is specific for a target sequence in a target genome, is exclusively expressed from the self-guiding integration construct; with the proviso that the self-guiding integration construct does not comprise an expression construct encoding a polynucleotide-guided genome editing enzyme.

13. A construct according to claim 11, wherein the self-guiding integration construct is a linear self-guiding integration construct.

14. A construct according to claim 11, wherein the self-guiding integration construct further comprises an additional polynucleotide element, wherein the donor polynucleotide optionally is a control sequence, a marker, a gene of interest, or a disruption construct.

15. A construct according to claim 11, wherein the functional guide-RNA, or part hereof, is encoded by a polynucleotide on the guide-RNA expression cassette and said polynucleotide is operably linked to an RNA polymerase II promoter, to an RNA polymerase III promoter or to a single-subunit DNA-dependent RNA polymerase promoter, optionally a viral single-subunit DNA-dependent RNA polymerase promoter, optionally a T3, SP6, K11 or T7 RNA polymerase promoter, and optionally to a self-processing ribozyme.

16. An ex vivo method for production of a host cell, comprising introducing into the host cell a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, wherein in the host optionally a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct; with the proviso that the self-guiding integration construct does not comprise an expression construct encoding a polynucleotide-guided genome editing enzyme.

17. An ex vivo method for production of a host cell, comprising introducing into the host cell two or more polynucleotide members, wherein these members have sequence identity with each other which allows them to recombine in the host cell to yield a self-guiding integration construct comprising a guide-RNA expression cassette capable of expressing a functional guide-RNA, or a part thereof, that is specific for a target sequence in a target genome, wherein the part of the self-guiding integration construct comprising said guide-RNA expression cassette is flanked at its 5′-terminus by a first polynucleotide and at its 3′-terminus by a second polynucleotide, wherein said first and second polynucleotide have sequence identity with sequences flanking the target sequence in the target genome, wherein in the host optionally a functional polynucleotide-guided genome editing enzyme is present or is introduced, wherein the self-guiding integration construct integrates into the genome at the target site, and wherein the functional guide-RNA, or part thereof that is specific for a target sequence in a target genome, is exclusively expressed from the introduced self-guiding integration construct; with the proviso that the self-guiding integration construct does not comprise an expression construct encoding a polynucleotide-guided genome editing enzyme.

18. The ex vivo method according to claim 16, wherein the self-guiding integration construct is a linear self-guiding integration construct.

19. The ex vivo method according to claim 16, wherein the self-guiding integration construct further comprises an additional polynucleotide element, wherein the additional polynucleotide element optionally is a control sequence, a marker, a gene of interest, or a disruption construct.

20. The ex vivo method according to claim 16, wherein the functional guide-RNA, or the part thereof, is encoded by a polynucleotide on the guide-RNA expression cassette and said polynucleotide is operably linked to an RNA polymerase II promoter, to an RNA polymerase III promoter or to a single-subunit DNA-dependent RNA polymerase promoter, optionally a viral single-subunit DNA-dependent RNA polymerase promoter, optionally a T3, SP6, K11 or T7 RNA polymerase promoter, and optionally to a self-processing ribozyme.

21. The ex vivo method according to claim 16, wherein a library of a self-guiding integration constructs is introduced into a population of host cells.

22. The ex vivo method according to claim 16, further comprising determining whether and/or where the self-guiding integration construct has integrated.

23. The ex vivo method according to claim 22, wherein the determination is made by analysis of a gene product produced by the generated host cell, by using selective growth conditions.

24. A host cell according to claim 8, said cell comprising a polynucleotide encoding a compound of interest.

25. The host cell according to claim 24, expressing the compound of interest.

26. A method for the production of a compound of interest, comprising culturing the cell according to claim 24 under conditions conducive to production of the compound of interest, and, optionally, purifying or isolating the compound of interest.
